(19) United States
(12) Patent Application Publication
(10) Pub. No.: US 2018/0055406 A1
(43) Pub. Date: Mar. 1, 2018

(54) Electrophysiological Measurement and Stimulation within MRI Bore

(71) Applicants: Ranajay Mandal, West Lafayette, IN (US); Nishant Babaria, West Lafayette, IN (US); Zhongming Liu, West Lafayette, IN (US)

(72) Inventors: Ranajay Mandal, West Lafayette, IN (US); Nishant Babaria, West Lafayette, IN (US); Zhongming Liu, West Lafayette, IN (US)

(21) Appl. No.: 15/685,514

(22) Filed: Aug. 24, 2017

Related U.S. Application Data
(60) Provisional application No. 62/378,956, filed on Aug. 24, 2016.
(60) Provisional application No. 62/471,545, filed on Mar. 15, 2017.

(51) Int. Cl. A61B 5/055; A61N 1/36; A61N 5/06; A61B 5/0476; A61B 5/04; A61B 5/0402; A61B 5/00; G01R 33/30; G01R 33/48

(52) U.S. Cl. CPC A61B 5/055; A61N 1/36025; A61N 5/0622; A61B 5/0476; G01R 33/4806; A61B 5/0402; A61B 5/0006; G01R 33/30; G01R 33/4808; A61B 5/04008

(57) ABSTRACT

A system for measuring an electrophysiological (EP) signal of a subject, e.g., while the subject is in an MRI bore, includes antennas and circuitry to measure the EP signal; detect, using the antennas, magnetic-field changes due to MR operation; and isolate the EP measurement from resulting electrical transients. A control unit operates the detection circuitry to measure the EP signal at a time other than during the magnetic-field changes. A communication module transmits the EP signal via at least one of the one or more antennas. Some examples include a reference electrode to contact the body of a subject; a differential-pair to transmit a reference signal; and a converter at a measurement electrode to reconstruct the reference signal from the differential pair. Some examples provide an electrical or electromagnetic (e.g., optical) stimulus to tissues of a subject during a quiescent, non-readout MR period.

### CROSS-REFERENCE TO RELATED APPLICATIONS

This application is a nonprovisional application of, and claims priority to and the benefit of, U.S. Patent Application Ser. No. 62/378,956, filed Aug. 24, 2016, and entitled “Electrophysiological Measurement and Stimulation Within MRI Bore” (atty. docket no. P074-0062USP1), and U.S. Patent Application Ser. No. 62/471,545, filed Mar. 15, 2017, and entitled “Electrophysiological Measurement and Stimulation Within MRI Bore” (atty. docket no. P074-0066USP1), the entirety of each of which is incorporated herein by reference.

### STATEMENT OF FEDERALLY SPONSORED RESEARCH OR DEVELOPMENT

This invention was made with Government support under Contract No. 5R01MH104402 awarded by the National Institute of Mental Health of the National Institutes of Health. The government has certain rights in the invention.

### BRIEF DESCRIPTION OF THE DRAWINGS

Objects, features, and advantages of various examples will become more apparent when taken in conjunction with the following description and drawings wherein identical reference numerals have been used, where possible, to designate identical features that are common to the figures.

FIG. 1 is a graph of example resolution characteristics of various imaging techniques.

FIG. 2 shows graphical representations of magnetic distortion effects.

FIG. 3A shows a graphical representation of an example of Magnetic Resonance Imaging (MM) data and non-MRI data.

FIG. 3B shows simulated ECG data reconstructed according to techniques herein from the wirelessly-transmitted non-MRI data depicted in FIG. 3A.

FIG. 4 shows an example stimulation circuit configured to apply an electrophysiological stimulus, e.g., to muscle tissues, and related components.

FIG. 5 shows an example measurement circuit, e.g., for measuring electroencephalogram, electrocardiogram, or other electrophysiological (EP) data, and related components.

FIG. 6 shows example signal-switching and -processing circuitry, and example data.

FIG. 7 is a plot of measured data of a rat ECG in an MRI bore.

FIG. 8 shows data of measurements that were taken, and exhibits effects of example artifact-removal techniques described herein.

FIG. 9 shows an example modulation system and example modulated data in the time and frequency domains.

FIG. 10 shows a simulated example of EP-data transmission during an MR scan.

FIG. 11 shows an example modulation system using frequency modulation (FM), and example data.

FIG. 12 shows an example of FM demodulation of MRI and EP data.

FIG. 13A shows data of an example of the operation of an example stimulation unit.

FIG. 13B shows a graphical representation of an example user interface for controlling a stimulation unit.

FIG. 14 shows an example of the operation of an example stimulation unit.

FIG. 15 shows example wireless power harvesting techniques, and corresponding data of power generation through an MR electromagnetic field.

FIG. 16 shows an example wireless power harvesting technique, and corresponding data of power generation through an MR electromagnetic field.

FIG. 17 is a high-level diagram showing the components of a data-processing system according to various aspects.

FIG. 18 is a pulse-sequence diagram of an example MRI and EP readout sequence according to some examples.

FIG. 19 shows (left) an example mode of operation of various examples; and (right) example measurement apparatus useful with various examples.

FIG. 20A shows an example EP measurement circuit.

FIG. 20B shows a block diagram of an example gradient magnetic field detection unit, example input signals, and an example output triggering signal.

FIG. 21 shows example signals related to a variable gain circuit.

FIG. 22A shows example components and techniques for the measurement of EP data and reduction of EMI from the MR environment.

FIG. 22B shows more detail of example components shown in FIG. 22A.

FIG. 23 shows example electrical and electrophysiological signals that were recorded using an example system such as described herein.

FIG. 24 shows example data of gradient signals and corresponding triggering signals provided by a gradient-detection unit.

FIG. 25 shows example data of triggering signals.

FIG. 26 shows an example synchronized sampling technique.

FIG. 27 shows an example synchronized sampling technique.

FIG. 28 shows an example of non-MR data reconstruction from raw MR-imaging data.

FIG. 29 shows an example triggering sequence of example analog and variable gain modulation circuits.

FIG. 30 shows example simulated signals involved in recovery of an EP signal from a measured signal including gradient-induced artifacts.

FIG. 31 shows examples electrical and electrophysiological signals that were recorded using an example system such as described herein.

FIG. 32 shows an example of the operation of an example stimulation unit.

FIG. 33 shows an example of the operation of an example stimulation unit.

FIG. 34 shows example EP signals that were recorded using an example system such as described herein.

FIG. 35 shows example EP signals that were recorded using an example system such as described herein.

FIG. 36 shows example filtered EP signals determined based on signals recorded using an example system such as described herein.

FIG. 37 shows an example MR pulse sequence and timing parameters for measurement and during-readout transmission of non-MR data.

FIG. 38 shows an example MR pulse sequence diagram and timing parameters for measurement and post-readout transmission of non-MR data.

FIG. 39 shows a graphical representation of data collected using FM transmission of EP data during MRI.

FIG. 40 shows graphical representations of data collected using FM transmission of EP data during MRI.

FIG. 41 shows an example printed-circuit board (PCB) stackup for reducing EMI in the MR environment.

FIG. 42 shows example electrical and electrophysiological signals that were recorded using a tested configuration.

FIG. 43 shows an example wireless power harvesting circuit and an example gradient-detection circuit.

FIG. 44 shows data that was collected in two tested configurations.

FIG. 45 shows a histogram of data that was collected in two tested configurations.

FIG. 46 shows example power-harvesting and frequency-generation components.

The attached drawings are for purposes of illustration and are not necessarily to scale.

### DETAILED DESCRIPTION

Throughout this description, some aspects are described in terms that would ordinarily be implemented as software programs. Those skilled in the art will readily recognize that the equivalent of such software can also be constructed in hardware, firmware, or micro-code. The present description is directed in particular to algorithms and systems forming part of, or cooperating more directly with, systems and methods described herein. Aspects not specifically shown or described herein of such algorithms and systems, and hardware or software for producing and otherwise processing signals or data involved therewith, can be selected from systems, algorithms, components, and elements known in the art.

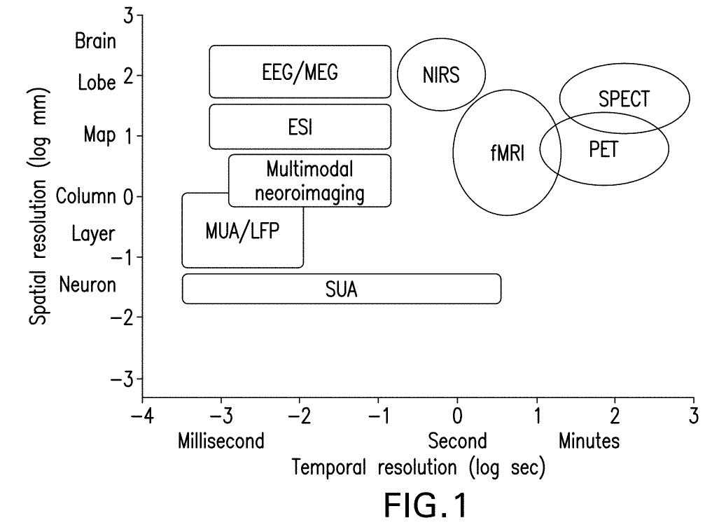

FIGS. 1-16, 41, and 43 show various examples of systems and techniques described herein, and related components. FIGS. 19-40, 42, 44, and 45 show structural and functional details of various examples, simulations of various examples, and data collected using techniques such as those described herein.

Non-invasive functional imaging tools, as a part of many clinical and research settings, have assisted understanding brain function and dynamics. Although the spatial and temporal resolution of different modalities have improved significantly over the past decade, major theoretical limitations on increasing resolution have motivated the need for integrating multiple complimentary neuroimaging modalities. Integration of these different modalities has opened new avenues to cross link brain activity across various spatial and temporal scales. Some examples include an MR-compatible, fully wireless system, capable of concurrent recording of electrophysiological (“EP”) signals such as electroencephalography (EEG), electrocorticogram, electrocardiogram (ECG or EKG), and neuromodulation (e.g deep-brain stimulation, optogenetic stimulation) within a Magnetic Resonance Imaging (MRI) scanner during image acquisition. The term “simultaneous acquisition” refers to this concurrent recording, and does not require that EP data and MR data be measured at precisely the same instant in time. Some examples interleave MR and EP measurements very quickly, e.g., more quickly than a biological process under observation undergoes substantial state changes. Various examples provide an effective and inexpensive alternative to bulky and complex conventional MR-recording systems. Example apparatus and software described herein can seamlessly interoperate with conventional MR-apparatus for multimodal brain imaging and stimulation applications.

The MRI scanner can be a very challenging environment for electrophysiological recordings (e.g. human EEG) during concurrent functional Magnetic Resonance Imaging (fMRI) acquisition. Conventional EEG systems use passive sensing with wired connections. However, the strong and time-varying MRI magnetic fields can provide challenges for conventional EEG systems with passive sensing and wired connections. The wires that connect electrodes to external amplifiers can form conductive loops, through which the magnetic flux varies dramatically due to rapid MR gradient switching, and involuntary electrode and head movements driven by cardiac pulsation, etc. As a result, the recorded electrical signals can suffer from severe electromagnetic induction artifacts, e.g., several orders of magnitude stronger than brain signals. Such electromagnetic interference can be problematic despite various signal processing techniques for retrospective correction. Moreover, some prior systems require MRI-compatible power supplies and amplifiers with a large dynamic range and a high sampling rate to fully sample and characterize the artifacts, rather than brain signals. Thus, the systems can be bulky and expensive when used in high-field MRI. For example, conducting materials within EEG recording system can affect electromagnetic fields within the MR environment and degrade the image quality significantly, in prior schemes. Some prior schemes depend on bulky and complicated shielding system or analog channel orientation.

Some examples herein provide sensing technology that significantly reduces the effects of electromagnetic induction on electrophysiological recordings. Some examples permit low-artifact and high-density human EEG (and animal LFP) recordings during concurrent fMRI acquisition. Some examples provide low-cost, high-density, and MR-safe EEG recordings with significant improvement in signal quality compared to some prior schemes. Various examples relate to an MR-Powered recording and stimulation system integrated with MR-hardware and acquisition software. Some examples relate to at least one of the following: EEG-fMRI, Multimodal Imaging, MR Power Harvesting, MR compatibility, MR-compatible recording system, MR-compatible stimulation system, Synchronized EEG sampling, Concurrent EEG-fMRI recording system, MR sequence, EEG, ECG, electrical stimulation, optical stimulation. Various examples pertain to the field of electrical engineering and manipulation of electromagnetic field and radiofrequency within a Magnetic Resonance Imaging or Spectrometer scanner. Some examples relate to biomedical instrumentation and imaging, e.g., examples integrating different aspects of physiological recording and stimulation with imaging.

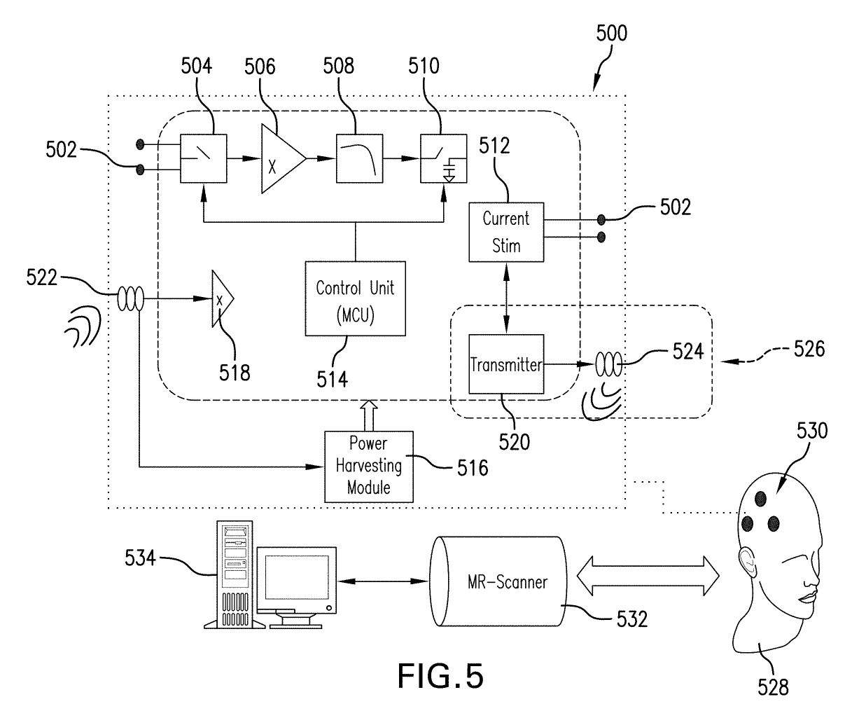

FIG. 5 shows an example measurement system 500 and environment, and related components. A subject 528 is positioned within the bore of an MR scanner 532, e.g., an MRI machine. System 500, which can also be placed within the bore of MR scanner 532, records EP signals from subject 528 concurrently with MR imaging of subject 528. System 500 includes communication module 526, which wirelessly transmits data of the collected EP signals to receivers in MR-Scanner 532, e.g., the same receivers that receive the MR data. The EP signals are then extracted from the received data and can be presented, e.g., in real time as the scan progresses, or after the scan. The EP signals can be presented together with, or separately from, the MR signals. As used herein, the magnetic field in the bore of MR scanner 532 is described, for brevity and without limitation, as a magnetic field “around” system 500 placed in the bore of MR scanner 532.

Various examples use the electromagnetic fields and hardware present in a Magnetic Resonance Imaging (MRI) scanner for various electrical and electrophysiological (EEG, ECG, LFP etc.) signal recording, or for different methods of stimulation, during concurrent MR-imaging. Various examples harvest wireless energy from rapidly varying electromagnetic fields and supply power for recording and stimulation without interfering with concurrent fMRI acquisition. Various examples provide a miniaturized, battery-free, and wireless system. Various examples provide a post-processing method that enables high-density bio-potential recording and stimulation during MRI, MRS (Magnetic Resonance Spectroscopy) or fMRI (Functional Magnetic Resonance Imaging). Various examples use a discrete-time variable-sensitivity amplification technique to reduce effects of electromagnetic interference during imaging. Various examples use hardware of an MRI machine as a receiving system for other signals which can be of different origins (e.g., biological or non-biological).

Monitoring high fidelity electrical and electrophysiological signals during MR imaging can be useful for, e.g., MR-guided interventions. Moreover, integrated measurement of different electrophysiological signals (EEG, MEG etc.) during concurrent MR imaging can provide data for research into the dynamic nature of human body and brain, and can permit determining treatments. Stimulation and concurrent imaging provide new ways to visualize, e.g., large scale neural response to neuromodulation at fine spatial scales. However, concurrent recording and stimulation during MR image acquisition poses some significant challenges as the MRI apparatus provides a hostile environment for some electro-magnetic signal recording or stimulation techniques.

MRI is a commonly-used tool for non-invasive imaging in many clinical settings and various fields of research. Within the MR Scanner, it can be useful to acquire additional data apart from the electromagnetic signals coming out of the imaged subject (e.g., a human or animal subject). These additional signals can include, e.g., temperature, pressure and physical conditions within the scanner, measurements of a patient's health condition(s) during the scan time (e.g., ECG, Heart rate, Respiration rate etc.), or a patient's response(s) to or during particular task(s) (e.g., key strokes, hand movements, eye movements, etc.). Also, continuous acquisition of the imaging data along with some electrophysiological data sets (EEG, MEG etc.) can permit, e.g., localizing seizure onset zones or mapping brain connectivity.

However, the environment within the MRI scanner can affect electrical measurements significantly. This is due to (1) the presence of a high static magnetic field, (2) high energy Radio Frequency (RF) excitation, and (3) rapidly changing magnetic fields. For the first reason, the use of any ferromagnetic device is restricted within the scanner and only materials which are “MR-Safe” can be placed inside/close to static magnetic field. Due to the presence of intermittent high power RF excitation, material placed inside the MR scanner may require proper shielding, and electromagnetic heating within conductors can become an issue due to the induced eddy currents. Finally, one of the major bottlenecks of concurrent measurement of any additional electrical data is the changing magnetic field used for MR image acquisition. These magnetic field changes can induce electrode voltages that are sometimes orders of magnitude larger than the actual recorded electrophysiological signal.

Some examples relate to cardiac MR imaging (CMR). Accuracy of single or multiple-cardiac-phase MR images is correlated with the reliability of the Electrocardiogram signal (e.g., a 12 lead ECG). Integrative imaging studies such as concurrent EEG-fMRI involve acquiring electrocorticography (ECoG) or electroencephalography (EEG) signals during fMRI. These techniques, in combination with some example systems and techniques described herein, can provide, e.g., precise localization of epileptogenic seizures and underlying sources. Various examples provide a non-invasive tool to measure neural events and target therapeutic solutions, e.g., even in the presence of inter-subject variation in the brain dynamics of epileptogenic activity. Some prior neuroimaging schemes focused on EEG and fMRI signals recorded in different sessions due to the degradation of SNR in both EEG and fMRI data during concurrent acquisition. Some prior schemes for EEG-fMRI record physiological signals only during the electromagnetically quiescent periods of MR image acquisitions. However, this curtails the efficacy of multimodal imaging by reducing the temporal resolution considerably. Some prior schemes provide insufficient signal integrity and synchronization of acquired EEG and fMRI data sets to permit effectively conducting multimodal studies.

Various examples permit manipulation of neural activity. Various examples provide a combination of stimulation, recording, and high spatial resolution imaging, which can permit, e.g., brain mapping or understanding brain dynamics during perception, behavior, and cognition. Deep brain stimulation (DBS) can serve as an effect neurosurgical technique (e.g., in place of ablation) for treatments of many neurological and psychiatric diseases and disorders, like Parkinson's Disease (PD), obsessive-compulsive disorder (OCD), epilepsy, clinical depression and Alzheimer's Disease. Following the efficacy of DBS treatment in treating Parkinson's Disease, DBS of the subthalamic nucleus (STN) and globus pallidus internus (GPi) was approved for PD and for OCD by the Food and Drug Administration in (FDA) in 2003 and 2009, respectively. Various examples herein permit measuring effects of local neuromodulation on different brain regions and large-scale networks.

Some examples permit synchronizing the acquired non-MR data with the acquired MR images, as the correlation and integration of these two datasets can be useful, for example in case of EEG-fMRI measurements. Some examples use triggering circuitry that is MR-compatible (for proper operation). In some examples, all connections with the scanner are shielded and substantially electromagnetically quiescent within the operating frequency of the scanner (e.g., below the noise level of the scanner). Such connections can be made, e.g., using coaxial (coax) or micro-coax cable, twisted-pair cable, or other shielded or low-emission cable.

Some previous schemes for measuring EP signals were made through acquisition of RF signals via tuned quartz oscillators and measurement of electromagnetic field and temperature. However, some of those schemes do not permit concurrent measurement of MR data and small electrophysiological signals. In some examples herein, concurrent acquisition and analysis can achieve better performance both in temporal and spatial domains compared to that achieved individually through either EEG or fMRI alone.

The MR environment, especially the rapidly-changing magnetic field, can reduce the SNR of acquired electrophysiological signals. Some examples herein permit recording EP signals during periods other than quiescent periods during MR image acquisitions. Some examples provide synchronized recording of non-MR data during MR image acquisition. Some examples use analog circuitry and wireless telemetry systems to mitigate challenges described above. Some examples of a system herein provide a simple standalone non-MR signal recorder and continuous monitoring platform that is compatible with standard MR systems.

Various examples include a system for concurrent and synchronized recording of electrical, optical, or electromagnetic signals corresponding to both MR and non-MR data. Various examples also provide programmable optical or electrical stimulation synchronized with imaging data acquisition. Various examples include a sensor module wirelessly powered through the electromagnetic field present within the MR scanner. The recorded non-MR data can be wirelessly transmitted and received by hardware and circuitry present within the MR apparatus. Some example recording systems herein can be safely operated within the MR apparatus without negatively affecting the original functionality of the MR scanner. Various examples include a computing system, e.g., software, capable of processing MR and non-MR data acquired through the apparatus. Continuous monitoring of non-MR signals and stimulating parameters can be observed using this system.

FIG. 1 shows a schematic illustration of spatiotemporal resolution ranges of various invasive and non-invasive recording and multimodal imaging and experimental techniques. Some imaging techniques available to the scientific community encompass a broad extent of spatiotemporal ranges. Each of these tools couples with various biological, electrophysiological, chemical parameters of human body and brain. Nuclear ionizing scanning tools like X-Ray, Positron Emission Tomography (PET), and Single Photon Emission Tomography (SPECT) have been used widely in the field of medicine, as they achieve centimeter-range spatial resolution and also metabolic specificity. However, these nuclear medicine techniques fail to achieve temporal resolution better than a few minutes.

MRI is capable of achieving spatial and temporal resolution in the ranges of millimeters and seconds, respectively. As a non-invasive imaging system with no nuclear radiation affecting live tissue, fMRI has emerged as a widely-used tool for imaging in various fields of brain research.

Measuring electrical potentials and magnetic fields on the scalp through EEG and magnetoencephalography (MEG), respectively, provides the necessary temporal resolution (on the order of milliseconds) to study dynamic brain activity. Nevertheless, spatial resolution is highly affected by the imaging accuracy of the underlying current sources, as the electrical field generated on the scalp surface is a combination of dendritic currents generated by a group of neurons that fire in a quasi-synchronized way. Some prior schemes combine EEG and fMRI to address the bottlenecks imposed by either EEG or fMRI when applied alone. However, the above-noted electromagnetic interference provided by MR measurements can greatly reduce the signal-to-noise ratio (SNR) of the EEG measurements.

Some examples provide integration among existing neural imaging techniques, various types of electrophysiological recording systems, neural perturbation methods like deep-brain stimulation (DBS), or optogenetic stimulation. These types of multimodal techniques not only can help to elucidate coupling among different modalities, but also aid in visualizing brain dynamics across different spatiotemporal scales. Some examples herein overcome technical challenges associated with multimodal integrated systems (e.g., EEG+fMRI or DBS+fMRI) that have previously limited multimodal techniques.

Various examples relate to multimodal imaging (e.g., neuroimaging) carried out within the MR-environment. An example multimodal neuroimaging system combines different aspects of neural recording (EEG, SUA, etc.), neuromodulation (DBS, optogenetic stimulation, etc.), or other techniques. Various examples include an MR-compatible electrophysiological recording and neuromodulation system. The proposed system achieves improved performance levels and is also much more affordable than some prior schemes.

Concurrent recording of electrophysiological signals (e.g. EEG, MEG, ECG) during MR image acquisition poses challenges, as the MR imaging apparatus provides a hostile environment for recording any type of electromagnetic signal. Various examples overcome these challenges to provide the benefits of multimodal signal acquisition. Various examples use at least one of the below-described components in order to reduce RF and magnetic gradient (gradient) induced artifacts. These examples can have reduced requirements for post processing to visualize the acquired signal.

Various examples provide a method to integrate measurement of various types of electrical and electrophysiological signals on a single platform. Various examples use MR-scanner hardware as a receiver for the additional data, in addition to a receiver for the imaging data produced by the scanner itself. Various examples combine different types of stimulation techniques (e.g., electrical or optical) with concurrent recording and imaging. Example stimulation systems are capable of generating various kinds of patterns related to diverse biological applications.

Various examples mitigate electromagnetic artifacts generated by an MR-apparatus, permitting performing electrical recording inside the scanner. Various examples can operate in either bipolar or unipolar configurations. Various examples use active components, reduced cable length, and differential signal transmission to reduce electromagnetic interference and noise and to compensate for signal attenuation. Various examples include analog processing and a discrete time variable sensitivity amplifier system.

Various examples include an electromagnetic detection circuit, containing on-board pickup coil, amplification, and filtering circuit, to reliably detect the times when artifacts, discussed earlier, are present. Various examples sample analog signals during these times or otherwise avoid these transient artifacts.

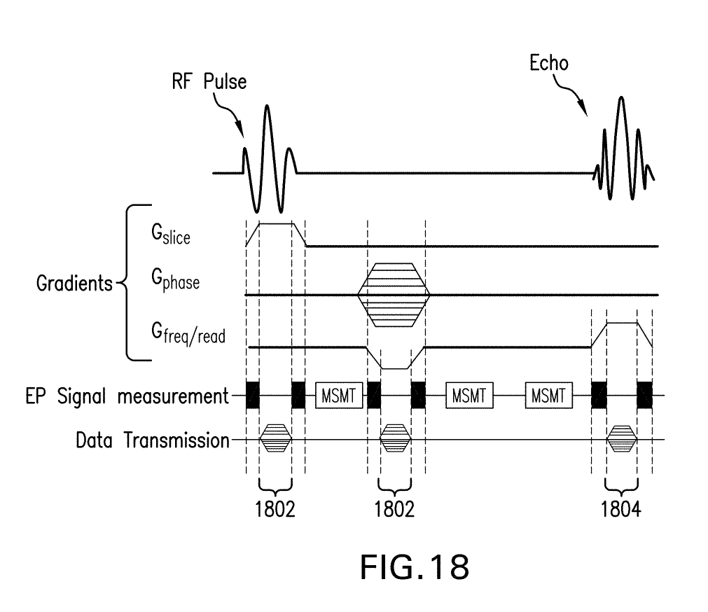

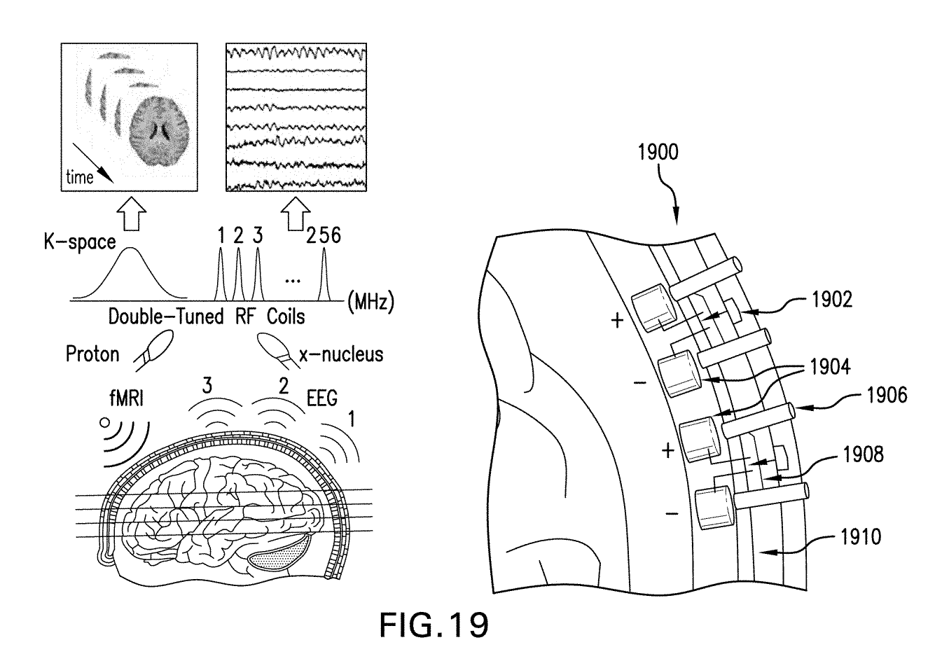

Various examples provide microsecond-level synchronization between MRI imaging data and other data measured in the MRI bore. By accurately controlling the modulation frequency of these additional datasets, various examples operate without negatively affecting the diagnostic capabilities of the MR-scanner. Various examples additionally or alternatively use a post-MR-readout pulse sequence to gather the additional data from other modalities on a conventional MR-scanner platform. Examples are discussed herein, e.g., with reference to FIGS. 18, 37, and 38.

Various examples include techniques for interpreting raw MRI data combined with the additional datasets. Various examples provide techniques for visualizing combined datasets in real time during MR imaging.

Various examples permit harvesting electromagnetic energy during concurrent MR-Imaging operation. The harvested energy from gradient magnetic field and RF energy can be used for powering a stimulator, recorder, or wireless transmitter.

Various examples can provide high-fidelity electrical and electrophysiological recording and stimulation during concurrent MRI (e.g., MRS or NMR) measurement. Gradient-triggered sampling and analog switching circuitry, combined with wireless reception of electrical signals by the MR coils, can provide increased signal integrity and reduced electromagnetic artifacts, while reducing the overall complexity by removing the dependence on bulky synchronization and shielding systems. Features or characteristics of some examples are listed below, marked (i)-(vii).

### Illustrative Feature (i)

Medical devices associated with the MRI apparatus can broadly be classified into two categories (a) MR-safe and (b) MR-compatible. Any piece of equipment to be used inside or near by the magnetic field of an MRI scanner should be MR-safe. MR-safe instruments do not pose any additional risk or hazard to the scanned subject or the apparatus itself but they may degrade the diagnostic information gathered by the imaging system. On the other hand, MR-compatible devices are not only MR-safe, but they also do not interfere with the imaging system or affect its functionality. For concurrent electrical signal recording and fMRI, MR-compatibility of the device can reduce artifacts that might otherwise degrade the SNR of signals as described herein.

Some examples of systems described herein exclude ferromagnetic materials. Non-ferromagnetic materials, such as aluminum and copper, can be used instead of ferromagnetic ones as conduction materials or connectors. Conducting loops within circuit boards can be reduced in size to reduce the induced voltages and circulating eddy currents that are caused by high-magnitude radiofrequency pulses during MR scanning. In some examples, electrodes are used that have reduced susceptibility to RF heating at MR-scanner frequencies. Instead of using discrete elements, some example electrode leads with more distributed resistances, e.g., commercially-available carbon-fiber wires, can be used to reduce specific absorption rate.

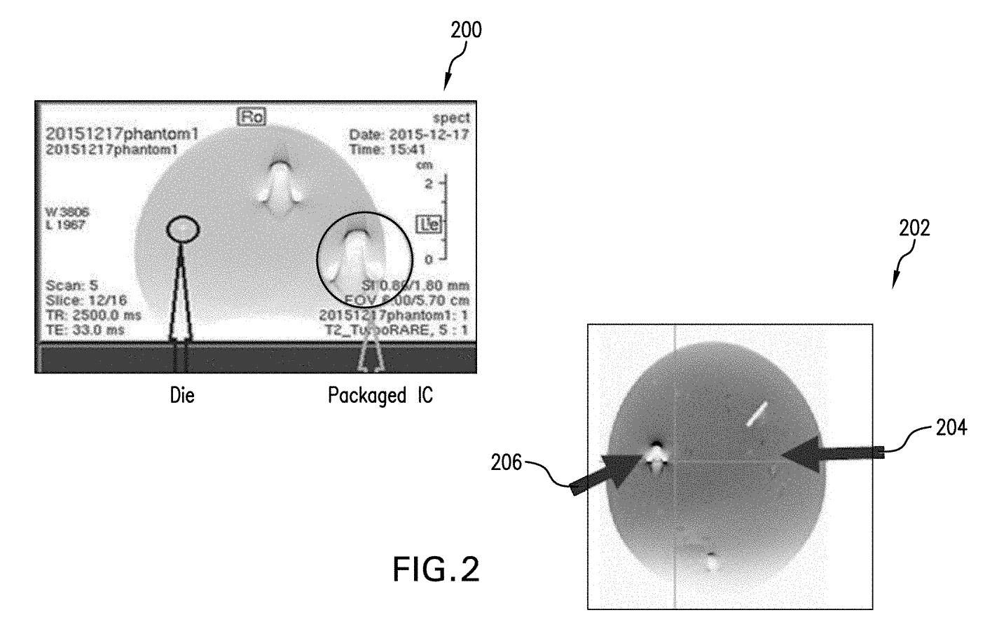

FIG. 2 shows a comparison of MR image distortion. Graph 200 shows distortion due to packaged and die-form ICs. Graph 202 shows distortion due to resistance of surface-mount non-magnetic discrete components 204 and magnetic discrete components 206.

In some examples, semiconductor materials used in integrated circuits can be MR-safe, but depending on the packaging type and manufacturing processes, components can interfere with the imaging system. This can be significant for components that are placed near the imaged surface. In some examples, individual assessment of each component is carried out to ensure MR-safety and compatibility. For example, integrated circuits (ICs) can be used without any commercial packaging (e.g., as known-good dice, KGD) to avoid any ferromagnetic components. Some examples include integration of discrete ICs according to processes such as those discussed herein. Some examples include specialized, MR-compatible IC packages. In some examples, quantitative analysis of electromagnetic compatibility can be carried out through numerical analysis, e.g., finite-difference time-domain (FDTD) analysis, to ensure MR-safety and compatibility.

Some examples include MR-safe and MR-compatible integrated-circuit packaging. For example, KGD can be wire-bonded to FR4 or other conventional printed circuit boards (PCBs), e.g., having rigid or flexible substrates, and overcoated with or otherwise encapsulated in conventional epoxies or other encapsulates for robustness. This is referred to herein as “die packaging.” The PCBs carrying the dice can then be packaged in non-ferromagnetic metal, plastic, or other cans or enclosures that are MR-safe and MR-compatible. The enclosures can include other components, e.g., coils described herein.

### Illustrative Feature (ii)

Three sources of noise can reduce the SNR of recorded EEG (or other EP) signals: (a) large static magnetic field (B₀), (b) strong Radio-Frequency interference (B₁), and (c) rapidly changing the gradient magnetic field (Gₓ, G_{y} and G_{z}). In some examples, changing magnetic fields create artifacts. For example, for humans, the peak gradient is generally about 70 mT/m. For small animals, a gradient of 200 mT/m is standard. Therefore, much larger artifacts can be present in small-animal tests than in human tests.

Depending on the scanning system, the static magnetic field can vary from the conventional 1.5 T to high fields of 10 T or more. These types of strong magnetic field can produce large artifacts as a result of small movements of the conductor or due to subject head motion. At least one of reduction of electrode length or custom designed head caps can be used, in various examples, to reduce such electrode and head movements.

Illustrative Feature (iii)

RF fields are used for the generation of electromagnetic signals from subjects inside the MR scanner during imaging, but such high frequency signals can cause significant difficulties during the recording of the electrophysiological signal. Demodulation and aliasing of RF pulses during MR-signal acquisition can produce artifacts in the order of 10² μV. The magnitude of these artifacts depends on the length of conductor used to carry the signals and especially the orientation of multiple channels within the transmission cables.

Gradient artifacts during concurrent fMRI and electrophysiological measurement may not be controlled by shielding in some examples, as conventional magnetic field shielding require ferromagnetic components that cannot be placed within MR environment. The static magnetic field is varied throughout the scanner bore in three directions (X, Y and Z) using specially designed gradient coils to provide spatial localization for individual voxels during electromagnetic signal acquisition by the receiver coils. The artifacts introduced by the gradient coils can have magnitudes proportional to the conductive loop sizes of the amplifiers. Artifacts, e.g., due to changes in the magnetic field around the device during the activation and deactivation of gradient coils, can achieve values from 10³ to 10⁴ μV, which in many cases is much higher than the small EP signals.

Some prior MR-compatible EEG recording systems neither block gradient signals nor attenuate noise at the acquisition stage, but instead amplify those signals or noise together with the acquired EEG (or other EP) signals. As a result, the SNR (e.g., signal-to-artifact ratio) remains very small. Moreover, the overlapping portions of the power spectra of the gradient artifacts and the EEG signals are very difficult to isolate, as conventional low-pass or bandpass filtering cannot be employed. As a result, the signal quality of higher-frequency EEG bands remains severely compromised for most of these recording systems. Advanced signal processing and adaptive noise cancellation techniques are still a necessity for all such systems. Moreover, some conventional recording systems do not provide a high enough sampling rate for acquisition of faster artifacts and EEG signals. Various examples herein provide RF or gradient artifact removal without requiring high-speed digitization systems.

### Illustrative Feature (iv)

The presence of switching magnetic fields and high RF deposition within the MR-bore requires every electronic circuit to be carefully designed to be Electromagnetically-Compatible (EMC). Various examples include methods for Electromagnetic Interference (EMI) reduction for the device such as at least one of: (1) Proper circuit design and PCB layout to minimize EMI radiation and common mode RF currents, (2) Specially designed power and grounding system, (3) Use of differential digital lines instead of analog signal transmission, or (4) RF filtering circuits to reduce EM deposition.

### Illustrative Feature (v)

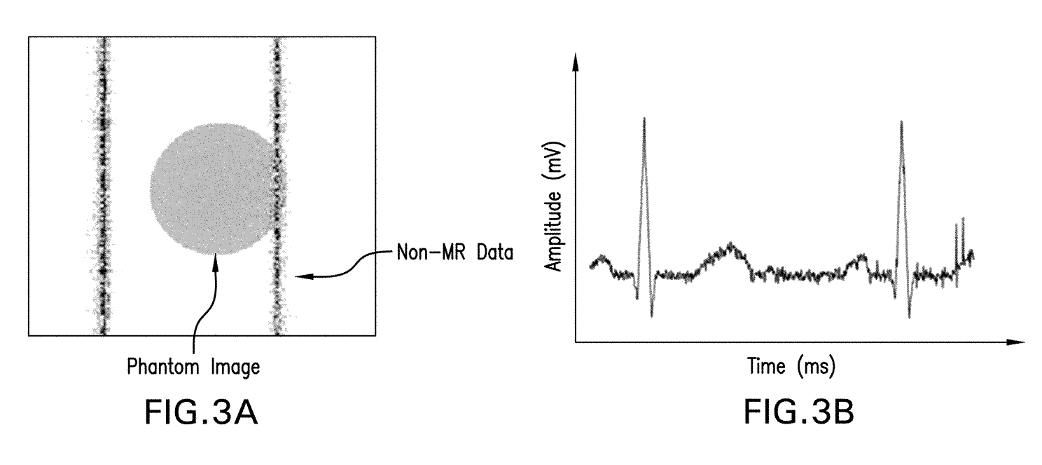

FIG. 3A shows an example encoding of non-MR signal in MR image using non-overlapping frequency bands. FIG. 3A was captured during a test in which non-MR data was sent from a signal source outside the MR bore to test wireless transmission.

FIG. 3B shows the non-MR data from the example of FIG. 3A, reconstructed into a simulated ECG signal.

In some examples, synchronization and time stamping of signals acquired from different modalities can be performed. To align the recorded signals from the different modalities, in some examples, electrophysiological signals can be measured within the MRI bore and the measurement of those EP signals can be synchronized with the fMRI image acquisition. In some prior schemes, such synchronization is achieved by sending the MR scanner clock to the digitization system for triggered sampling of amplified electrophysiological data. However, such prior schemes suffer from low SNR due to artifacts described above.

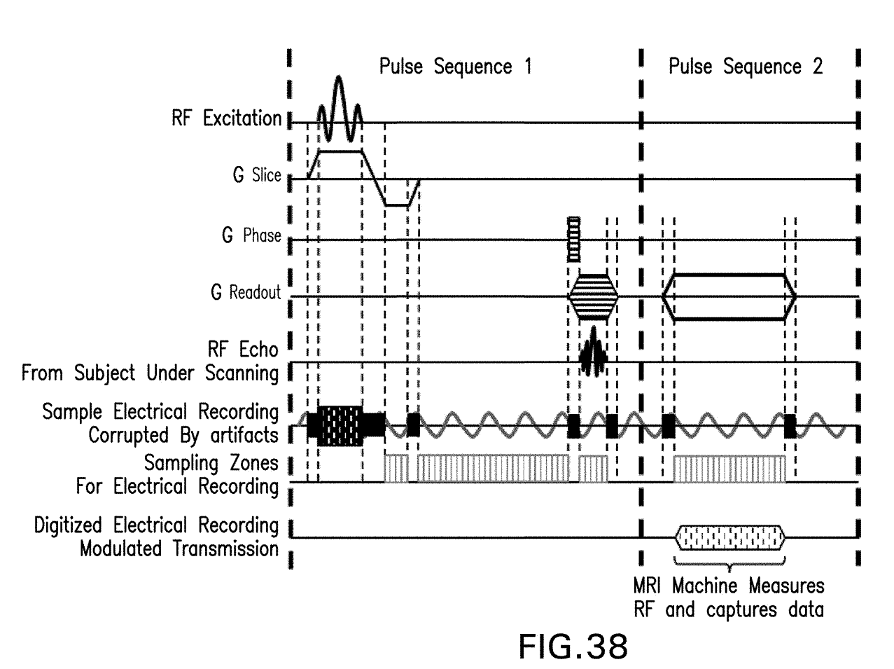

In some examples herein, integration and synchronization with the MR-scanner is achieved through wireless detection of gradient and RF pulses during an MR scanning (e.g., MRI) sequence. Some examples can transmit recorded electrophysiological data during the imaging process at distinct, non-overlapping (with respect to MR signals) frequency bands. These frequency bands do not interfere with the electromagnetic signals coming out from the subject being imaged, but are visible to the receiving coils within the MRI. As a result, the additional non-MR signals appear as lines within the MR-image, e.g., as depicted in FIG. 3A. Moreover, new MR pulse sequences can be designed to accommodate these electrophysiological signals (e.g., as discussed herein with reference to FIG. 38). Additionally or alternatively, an additional MR RF coil (e.g., a dual-tuned or broadband coil) may be tuned to operate in a different frequency range for EP signal reception, to substantially reduce the potential for interference with the frequency range for MR signal reception.

Some examples include MR software for proper identification and separation of these MR and non-MR data, or for concurrent visualization of the electrophysiological signals. In some examples, the electrical signals obtained from this method can be automatically synchronized with individual gradient change(s), as the same receiver coil is used for both applications and digitization of the acquired data is triggered using the gradient and RF detection circuit.

### Illustrative Feature (vi)

Energizing an EP-signal recording system, e.g., using powering circuitry or cables, can hinder the functionality of the MR-scanner. Some prior battery-powered recording systems require added magnetic and RF shielding, and the application of special materials is needed for the battery composition to make the system MR-safe. This increases the cost and complexity of such MR-compatible recording systems.

In some examples herein, the MR environment provides an opportunity for wireless power harvesting for electronic devices due to the presence of the varying magnetic field and strong RF excitation. Example systems can include a wireless power harvesting module that extracts power utilizing the RF excitation and also the magnetic field change due to the gradients during image acquisition. Some examples include miniaturized coils that can harvest energy from MR electromagnetic fields.

Illustrative Feature (vii)

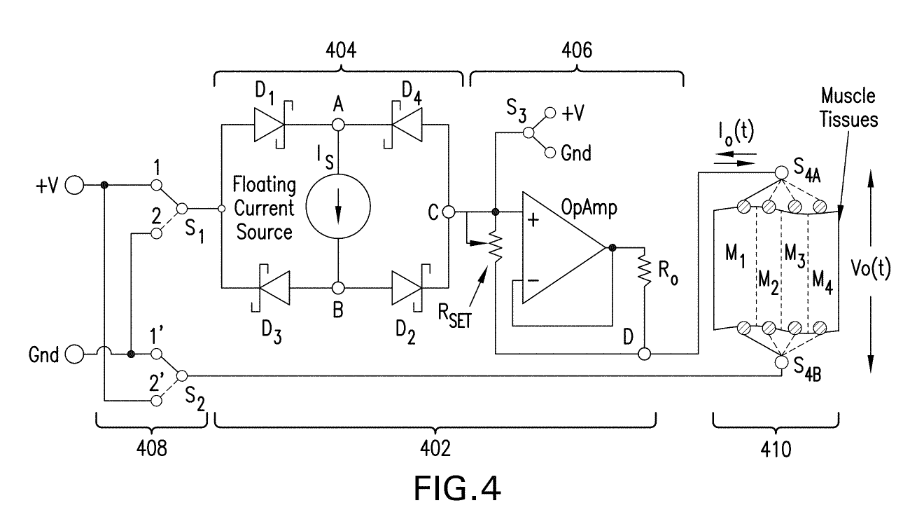

FIG. 4 shows an example schematic of a low power, bi-phasic current stimulation neuromodulation module, and related components and tissues. The illustrated system can provide a stimulation current of roughly 0.1-20 mA, a charge balance of up to 2 pC, a max pulse frequency of up to 800 pulses per second, a quiescent power of up to 900 and 4 independent channels, in some examples. Also depicted, merely for clarity of explanation, are muscle tissues Mn being stimulated by the circuit. The muscle tissues are not part of the stimulation system. Muscle tissue is a nonlimiting example, and other biological tissues can be stimulated using the depicted system. In the illustrated example, the hatched circles at the right represent electrode contacts, e.g., biopotential electrodes interfacing between ionic and electronic conduction. In some examples, at least one electrode contact is part of the system; in other examples, the electrode contacts are separate from the system but communicatively connectable thereto.

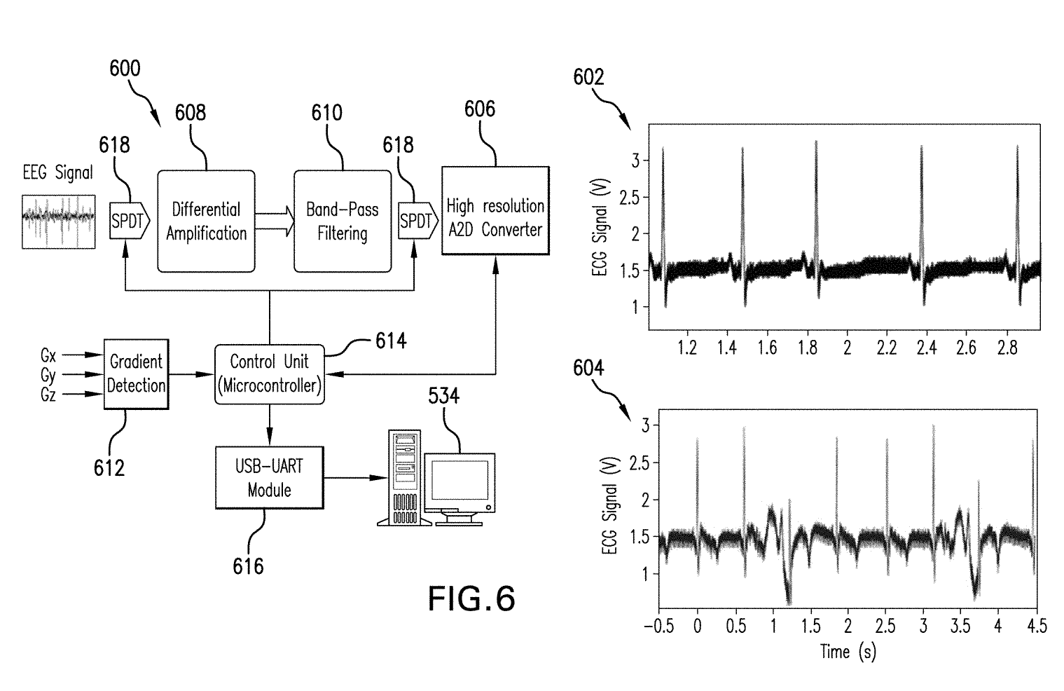

Various examples include MR-compatible, wirelessly-powered neuro-stimulators. Various examples include a low power neuromodulation system that integrates with wireless recording systems, e.g., shown in FIGS. 5 and 6. Various examples can independently provide current or optical stimulation (e.g., application of electromagnetic radiation to a subject) at variable frequency and amplitude.

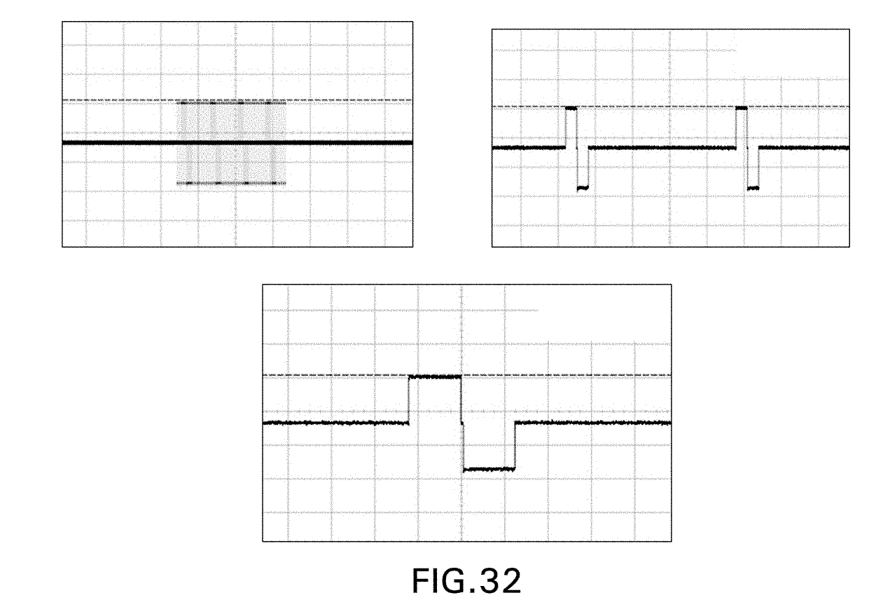

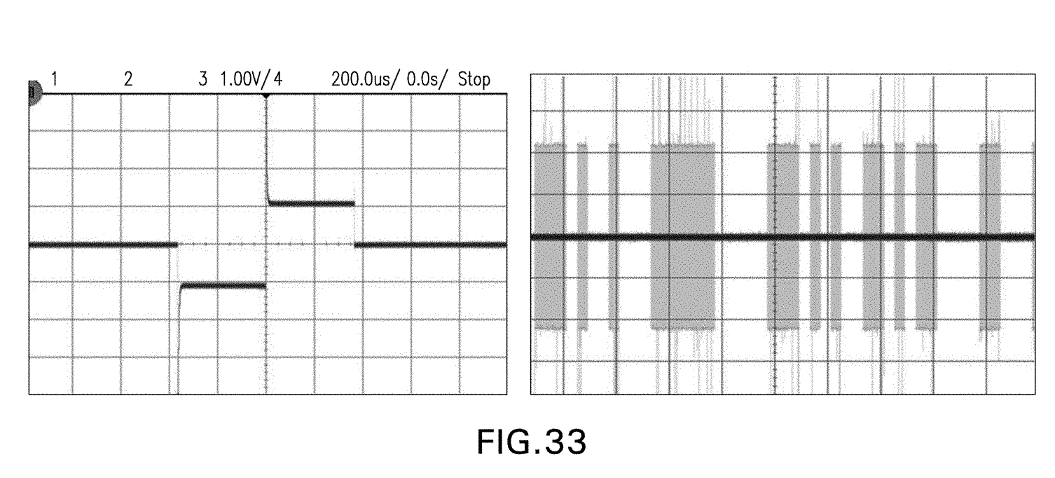

Stimulator 402 can comprise a reference bi-phasic current generator 404 and an adjustable current up-scaler 406 where an op-amp adjusts the current (gain >1 or gain <1 are both available). Direction switches 408 S1-S3, S4A, and S4B change the direction of stimulation, e.g., to alternate the direction of current flow and reduce charge buildup that might otherwise damage tissue. S1-S3, S4A, or S4B can be analog switches. Also attached to the programmable bi-phasic stimulator is an electrode selector and biological load impedance 410. The stimulation sequence can be pre-programmed or downloaded at runtime (e.g., #8). Additionally or alternatively, parameters can be downloaded and used to customize a pre-programmed sequence. Parameters can include pulse width, pulse frequency, current (Iₛₜᵢₘ), direction of current (including switch settings), and charge balance. Charge balance can represent the mismatch in charge when switching current directions. In some examples, the current has the same magnitude in both directions. Various examples of stimulation are discussed herein with reference to FIGS. 32 and 33.

Some examples include at least one stimulation unit, e.g., as discussed herein with reference to FIG. 4, 13, 14, 32, or 33. The stimulator can include die-packaged analog components to reduce noise. In some examples, circuitry can be reduced in size, e.g., via chip-scale packaging, to reduce interference with the imaging system. Stimulation units can, e.g., provide at least one of electrical current, electromagnetic radiation (e.g., light, infrared, ultraviolet, or other EM), or other forms of energy to tissue of a subject. The electrical current can be used for, e.g., muscle or deep-brain stimulation. The electromagnetic radiation can be used for, e.g., optogenetic stimulation. Various examples include MR-compatible wirelessly powered neuro-stimulators.

### Illustrative Feature Combinations

Various examples use at least one of three subsystems, designated (1)-(3), to reduce RF- and gradient-induced artifacts, or to reduce the required post-processing.

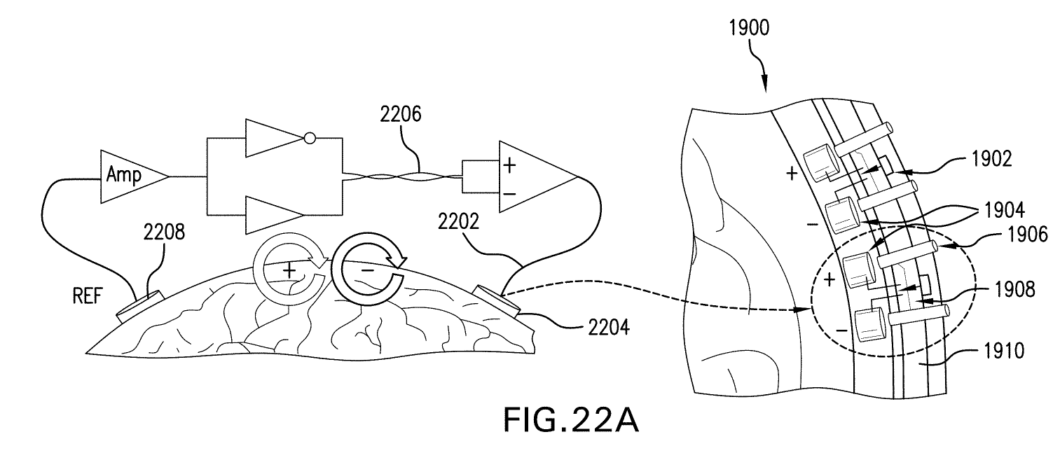

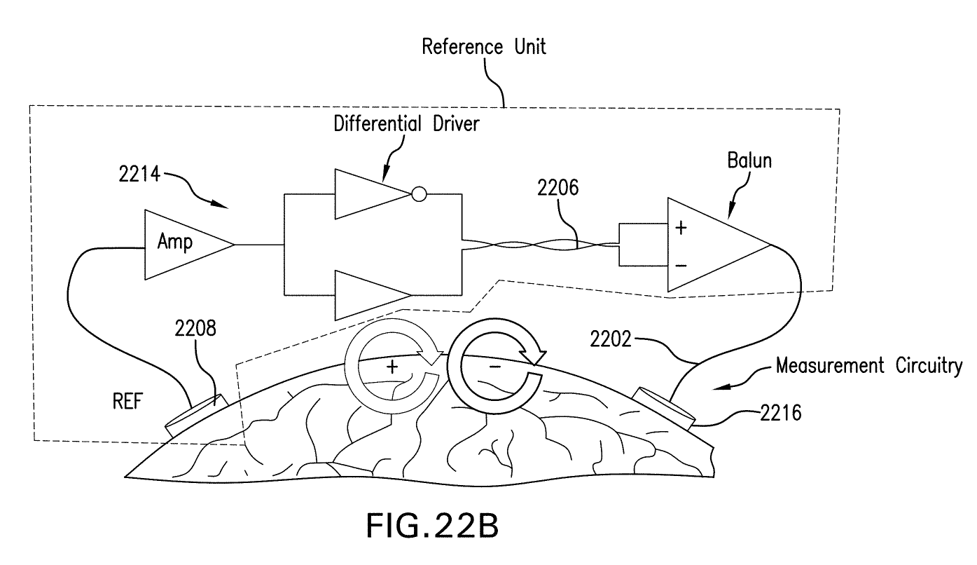

(1) Recording leads and cables: As both RF and gradient artifacts are dependent on the analog loop size before amplification, in some examples, montages of common reference or twisted electrode pairs can be used to reduce such artifacts. A “montage” is a particular configuration of orientation and harnessing of lead wires or other wires used for EP-signal detection. In some examples, the recording lead length can be reduced by placing the amplification and digitization system within the MR-bore. Some examples include a miniaturized system that sits close to the recording surface and reduces the cable length significantly. Examples are discussed herein, e.g., with reference to FIGS. 22A and 22B.

In some examples, the amplifier, filter, and digitizer are placed within the MRI bore, e.g., adjacent to the signal source (e.g., the subject's head, arm, or chest). This can reduce the analog loop size before amplification and filtering, thus reducing movement artifacts caused by the subject or other artifacts. See, e.g., FIG. 22A. The cables can further be shielded using a non-ferromagnetic material in the effort to further attenuate the RF- or gradient-induced artifacts or heating.

(2) Gradient and RF-triggered analog switching circuit: Gradient artifacts are the most prevalent noise in multimodal imaging. Some examples include coil(s) to pick up the magnetic field change during imaging. A switching circuit blocks analog signals during artifacts and keeps the amplifier unsaturated. Examples are discussed herein, e.g., with reference to FIG. 5, 6, 17, 20A, 20B, 21, 24-26, 29, 30, 31, 37, 38, or 43.

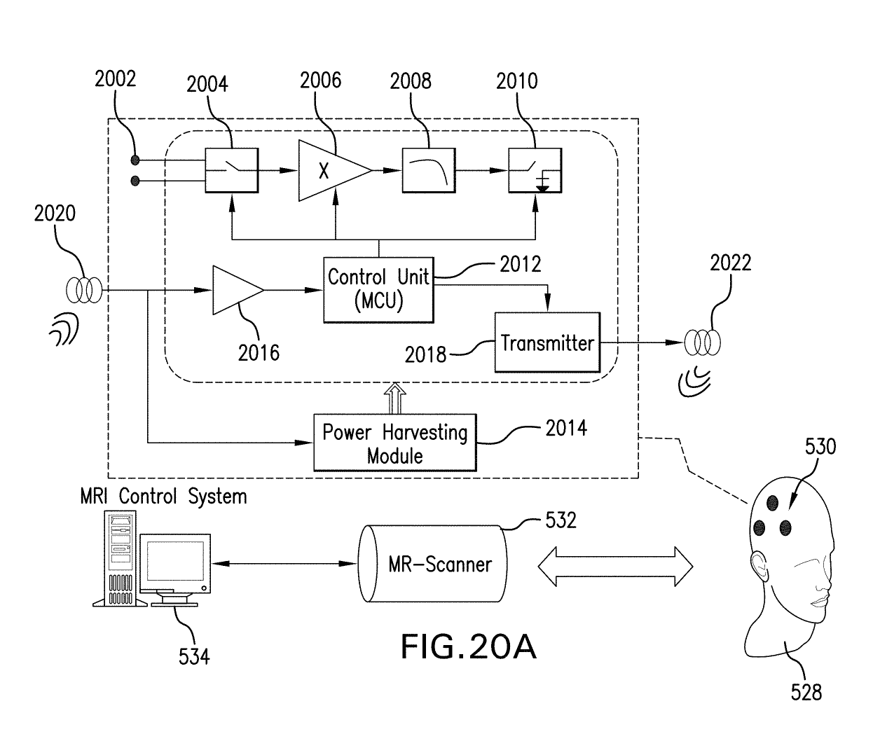

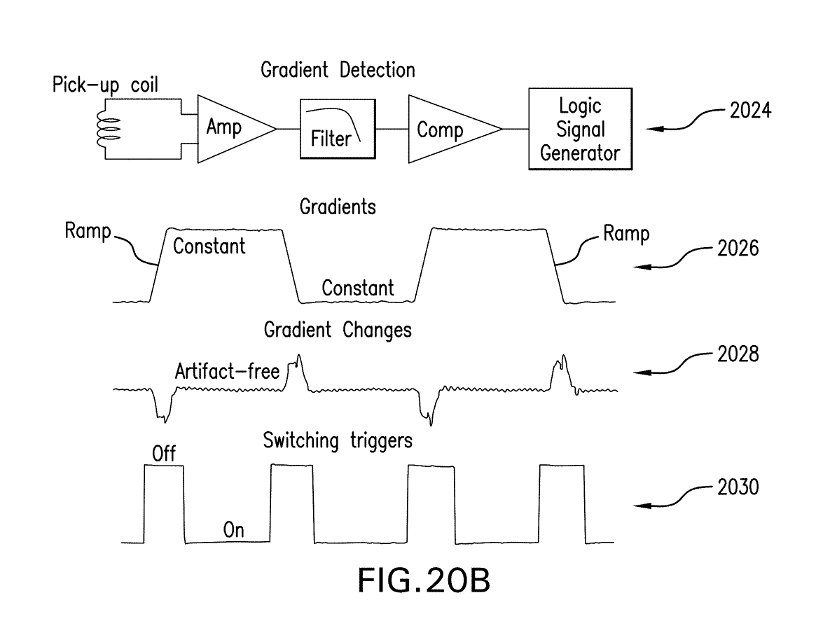

RF and gradient pulses during MR scanning can be detected by tuned pick-up coils or power-harvesting coils (described in FIG. 20B) to detect changes in the magnetic-field based on currents flowing in those coils. Multiple coils can be used together to measure the magnitude and direction of a net magnetic-field gradient in the MRI bore. The signal is filtered and amplified before being converted into a binary output. Example gradient detection circuitry can provide other systems with an indication of when the RF or gradient pulses are present or absent (e.g., as defined with respect to a predetermined noise threshold). This can permit measuring EP signals or transmitting data of measured EP signals at times when gradients will not unduly impair the measurements or transmissions.

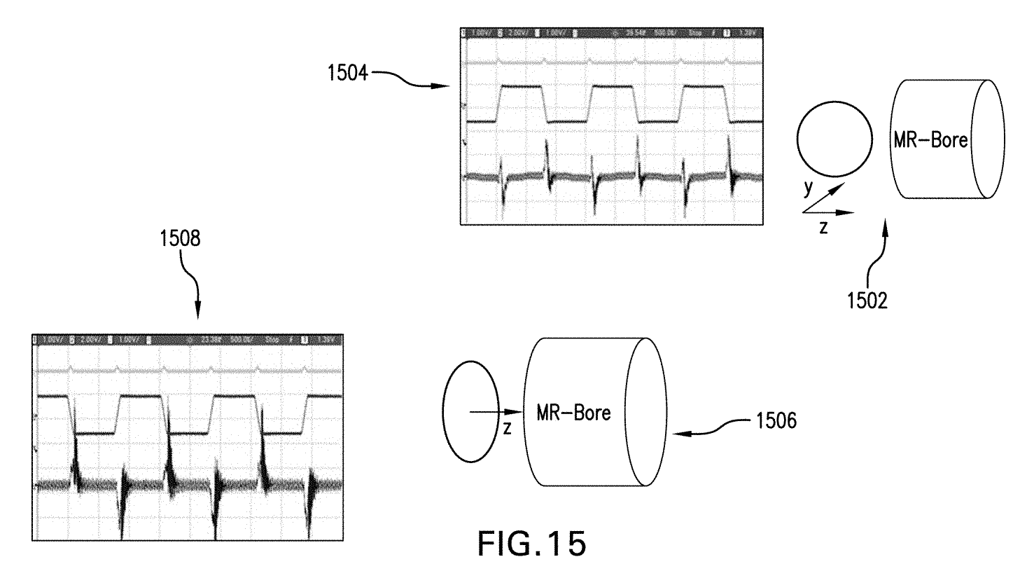

Example pulse sequences are shown in FIGS. 18, 37, and 38. Gradient pulses take on a variety of profiles based on the type of sequence. The magnetic field can be changed, e.g., according to a trapezoidal profile (gradient echo) of magnetic-field strength (in, e.g., mT/m) or of current (in, e.g., A) as a function of time. Herein, “activation” and “deactivation” of a gradient coil refer to ramps up or down in magnitude of a current through that gradient coil. Any number ≧1 of coils can be used. Example coil waveforms are shown in FIG. 15, 16, 26, or 30.

(3) Adaptive sampling: Using a high dynamic-range analog amplification circuit, the contribution of individual gradient changes can be precisely identified, and sampling can be performed at selective portions. Examples are discussed herein, e.g., with reference to FIG. 5, 6, 18, 20A, 24-26, 37, or 38. In some examples, low-power, high-speed switching circuitry can be combined with low-power amplification and filtering circuitry in an analog processing circuitry block.

In some examples using any of (1)-(3), signal processing can be performed as described herein. In some examples, retrospective signal processing methods are used to remove the RF and gradient artifacts. These postprocessing methods can include, e.g., Averaged Artifact Suppression (AAS) or Median Filtering.

Some examples can be used with many different multimodal imaging or MR-guided interventions carried out within the MR-environment. Various examples permit combining different aspects of electrical and electrophysiological recording (EEG, SUA etc.), neuromodulation (DBS, optogenetic stimulation, etc.). Various examples provide an MR-compatible electrophysiological recording and neuromodulation system. Various examples do not suffer from bottlenecks present in some prior schemes.

### Illustrative Configurations

FIG. 5 shows an example diagram of a wireless recording, stimulation, or neuromodulation System 500 integrated with an MR-Apparatus. As shown, the illustrated components of system 500 interact with a subject 528 (represented graphically as a head with black dots representing electrodes 530) in an MRI scanner 532 (“MR-Scanner”) controlled by an MRI Control System 534. The depicted subject is not part of the depicted system, and is shown merely for clarity of illustration. In some examples, the electrodes 530 are part of the illustrated system; in other examples, the illustrated system is communicatively connectable with the electrodes 530, e.g., via electrodes 502. In some examples, electrodes 530 can represent electrodes 502, or vice versa.

An example system includes component(s) belonging to at least one of the following component categories: MR-compatible electrodes 502 for stimulation and recording; analog switching circuit 504 for blocking of MR-artifacts; analog amplification and processing circuit 506, 608 for amplifying or filtering relatively small (compared to the MRI-induced artifacts) electrophysiological (EP) signals; filtering block 508, 610, e.g., a bandpass or other filter, for filtering the amplified electrophysiological signals; high dynamic range analog to digital converter 510 to accommodate minute signal variation; programmable bi-phasic current stimulator (“stimulation unit”) 512 for variable stimulation parameters (e.g. amplitude, frequency) (FIG. 6); microcontroller (MCU) 514 for bidirectional telemetry, stimulation parameter selection, controlling analog switching circuit, and synchronized sampling; wireless power harvesting module 516; gradient detection circuitry 518, 612 for extracting power and gradient field change information from MR environment; transmitting module 520 for bi-directional telemetry; wireless power harvesting antenna 522; and data transmitting antenna 524. The antennas 522, 524 can be or include, e.g., conductive coil antennas or other antenna configurations. A diagram of an example system is depicted in FIG. 5. The USB-UART module in FIG. 6 can be used, e.g., for testing or production. Some examples communicate data wirelessly. Filtering block 508 is depicted as a low-pass filter for clarity of the diagram, but is not limited to that depiction.

FIG. 5 and FIG. 6 show example systems 500 & 600, according to various examples. Various examples include at least one of the blocks described below, marked #1-#8, or at least one of blocks 502-516. Various examples include at least one of each of the following: digital conversion block 510; control unit 514, 614; communication module block 520; and energy-harvesting block 516. Various examples additionally include at least one stimulation module 512, or at least one detection module 518. A stimulation module 512 or a detection module 518 can be accompanied by an analog switching 504, amplification 506, and filtering block 508. A “channel” refers to a pair of electrodes 502 used for EP stimulation, or to an electrode or electrode pair 502 used for EP detection. Various examples can include zero, one, or more stimulation channels, or zero, one, or more detection channels, in any combination that includes at least one of a stimulation channel or a detection channel. In some examples, each of control 514, communication 520, and energy-harvesting 516 blocks is connected with more than one channel 502. In some examples, A/D conversion can be handled by an analog-to-digital converter (ADC) 606 per channel, or a multichannel ADC 606, or any combination thereof. Various examples provide a USB-UART module 616 or other debug, control, or programming interface connectable with, e.g., a computer outside the MRI bore, such as control system 534.

FIG. 6 shows an example implementation 600 of analog switching and processing. Measured data are also shown of a Rat ECG outside of the MR-Bore 602 and inside the MR-Bore 604 where the static magnetic field effect is recorded without imaging.

### Illustrative Feature #1

Referring back to FIG. 5 and still referring to FIG. 6, there is shown an example of a digital conversion block including a low power triggered converter for digitization of analog signals. Examples include digital conversion block 510 and ADC 606. Illustrative Feature #1 can include components described herein with reference to Illustrative Features (i), (iii), (iv), or (v). Some examples include a discrete, e.g., off-the-shelf, die-packaged ADC 606. In some examples, an ADC 606 is used having, e.g., a 12-, 14-, 16-, or 24-bit resolution per channel.

Some examples herein include analog recording circuitry incorporating gradient- and RF-pulse avoidance systems discussed herein and configured to capture the EP signal of interest. Some example analog circuitry operates in the frequency range of 1 Hz-10 kHz. Some examples include a high pass filter, e.g., of filtering block 508 or 610, having a pole or break frequency at 1 Hz to reduce DC offset and DC swing of the EP signal. On the other end of the frequency range, one, or a series of, low pass filter(s), e.g., of filtering block 508, can be used to reduce high frequency noise, e.g., above 10 kHz. EP signals falling in the frequency spectrum delimited by filtering block 508 or 610 can be captured. In some examples, this frequency range can be narrowed to a specific region in the spectrum to only capture certain types of electrical or electrophysiological signals, such as EKG, LFP, EEG, etc. This can be done by changing the configuration of filtering block 508 or 610, in some examples.

The analog recording system can include an amplification stage to properly amplify the EP signal for digitization, transmission, and visualization. The amplification stage can be implemented using amplification block 506 ahead of filtering block 508 or 610, or using an amplifier after filtering block 508 or 610. A gain of 1028.8 Vout/Vin (60.25 dB) can be applied to the signal over the series of analog stages when gradient or RF pulses are not present (refer to gain switching section under Illustrative Feature #7) and an attenuation of 0.002 Vout/Vin (−54 dB) can be applied when the gradient detection circuit encounters a fluctuation in the magnetic field, in some examples. The gain and attenuation can be selected to capture the signal of interest for the species being monitored. In some examples, 60.25 dB/−54 dB was used to monitor the EKG and local field potential in a rat.

After analog processing, the filtered, amplified signal can be digitized through a 12-bit (or other) analog to digital converter 510, 606. In some examples, the preprocessed analog signal can be sampled at 1.33 kHz while implementing synchronized sampling, discussed herein. In some examples, parameters such as sampling resolution, sampling timings, and sampling rates can be selected based on the EP signal or the species.

### Illustrative Feature #2

The control unit 614 can include a low power processor for controlling onboard system work flow. Examples are discussed herein, e.g., with reference to FIGS. 5, 6, and 17, e.g., MCU 514 or processor 1786. A conventional microcontroller can be used provided it is die-packaged or otherwise MR-safe and -compatible. The control unit 614 can be configured, e.g., programmed, to perform functions described herein. Illustrative Feature #2 can include components described herein with reference to Illustrative Features (i), (iv), or (vi).

### Illustrative Feature #3

The Communication Module can include wireless transmission of non-MR data in frequency bands received by the scanner, e.g., communication module 526 including transmitting module 520 and data transmitting antenna 524, or USB-UART module 616. The communication module can additionally or alternatively receive control signals regarding stimulation and recording cycles from control system 534, e.g., in response to commands from a user of control system 534. Examples are discussed herein with reference to FIGS. 5, 6, and 9-12, 17, 18, 209A, 37, and 38. Illustrative Feature #3 can include components described herein with reference to Illustrative Features (i), (iii), (iv), or (v).

In some examples, the transmitter carrier frequency can be 300.35 MHz, and the scanner bandwidth can be 333 kHz. This can permit both imaging data and non-imaging data to be captured by the MR-receiver coils (e.g., of a BRUKER 7T animal MRI machine).

The analog EP signal from #1 (e.g., from filter 508, 610, or 2008), or the digitized counterpart thereof (e.g., from ADC 510 or 2010), can be provided to an RF transmitter system 526 that transmits the data at frequencies detectable by the MR-Receiver coil. In some examples, the PLL based transmitter generates these transmitting frequencies from a reference frequency generator. In some examples, the reference frequency generator is a crystal resonator or dedicated integrated circuit (e.g., a PLL or frequency detector) that generates this reference frequency from RF excitation of the MR scanner.

In some examples, the communication module transmits data via the power-harvesting coils 522 (#4 below) or dedicated communication coils 524. In some examples, the communication module transmits on a frequency the MRI readout coil is configured to receive (e.g., as discussed herein with reference to FIG. 19). This advantageously reduces the number of parts required on the device, and permits concurrently capturing MRI and electrophysiological (EP) data (e.g., FIGS. 3, 9-12). In some examples, the device includes exactly one transceiver: the MRI-readout-frequency transceiver. In other examples, the device includes at least two transceivers. Transceivers can be connected to respective antennas of the device or to the same antenna. In some examples, the communication module can communicate using other wireless protocols, e.g., BLUETOOTH or WIFI.

In some examples, at least one of synchronization or time stamping of signals acquired from different modalities can be performed. To align the recorded signals, electrophysiological signals can be measured within the MRI bore, and digitization of the EP signals can be synchronized with the fMRI image acquisition. This can permit triggering based on time bases other than the MR scanner clock, which can provide increased flexibility in taking physiologically-pertinent measurements.

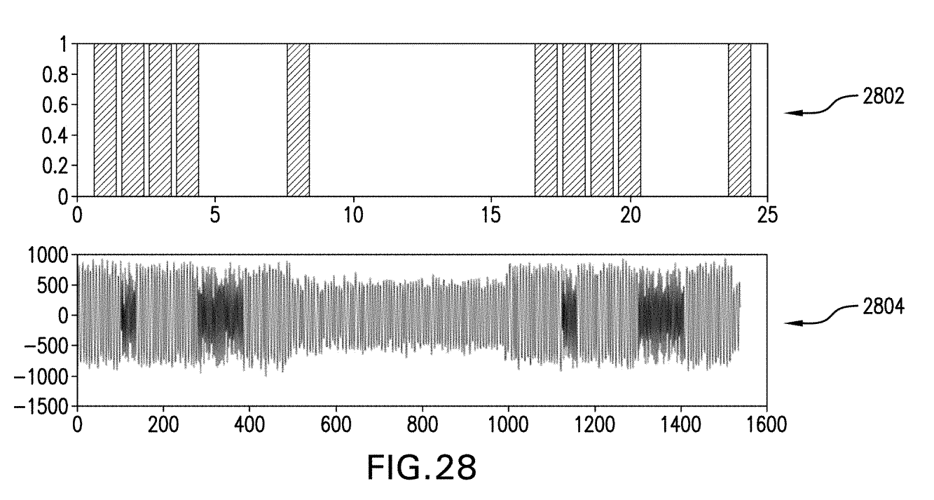

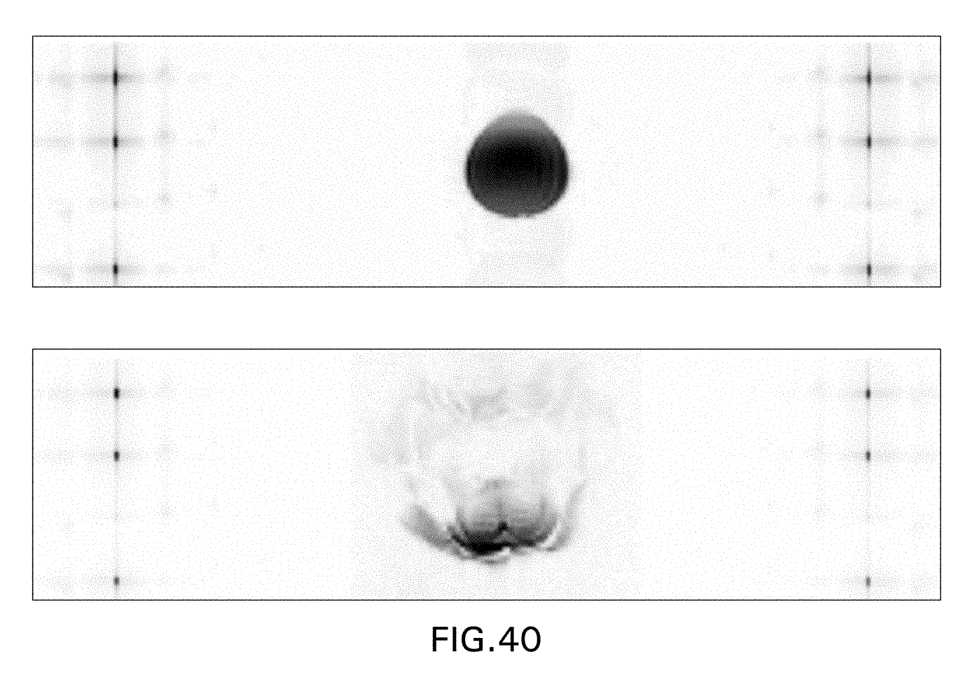

In some examples, herein, integration and synchronization of EP measurement with the MR-scanner is achieved through wireless detection of gradient or RF pulses during any imaging sequence (e.g., FIG. 5, 18, 37, or 38). Some examples transmit recorded EP data during the imaging process at distinct, non-overlapping frequency bands. These frequency bands do not interfere with the electromagnetic signals coming out from the subject being imaged, but are visible to the receiving coils within the MRI (e.g., to double-tuned MR receiving coils in an extended FOV configuration, discussed below with reference to FIG. 19). As a result, the additional non-MR signals appear as lines within the MR-image as depicted in FIG. 3, 11, 39, or 40. FIG. 40 shows an example encoding of non-MR signal in MR image using non-overlapping frequency bands. FIG. 28 shows an example of demodulation of non-MR data to acquire an original signal, depicted in FIG. 28 as a simulated ECG signal.

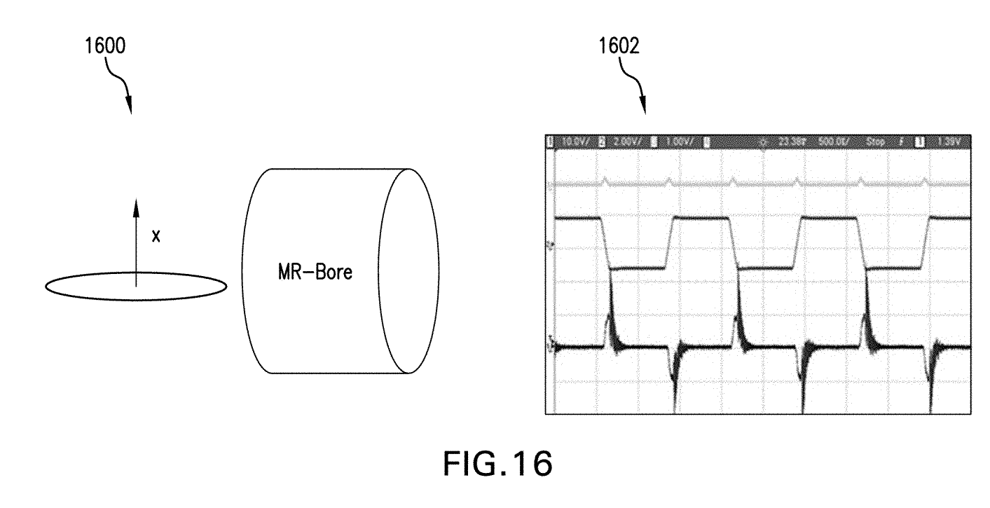

In various examples, the system transmits data whenever a gradient coil is active, as detected by the detection circuitry 518 (#6, #7 below) (see, e.g., FIG. 16, 20B, or 43). In some examples, an EP measurement system as described herein can operate without data of the exact MRI sequence. Sending whenever the gradient coils are active, regardless of whether the MRI is reading out data, will permit the MRI to receive the data without requiring the MRI to communicate to the EP measurement device (although the MRI can communicate to the device, e.g., as discussed herein with reference to #8 below). The device can be programmed with details of a particular MRI sequence, but that is not required. In some examples, the device (#6 below) is programmed to detect activation of the gradient coils, e.g., based on a trapezoidal or other predetermined activation profile. The device can transmit during the plateau of any detected trapezoid. Examples are discussed herein, e.g., with reference to FIG. 20B, 37, or 38.

Gradient detection (#6, below) determines when the readout gradient is active, and a signal indicating that determination can trigger transmission. Some examples are independent of MRI pulse sequence, but pulse sequence can be programmed in if desired. Many MRI machines use the same pulse sequence, e.g., trapezoidal profiles on the gradient coils (e.g., FIG. 18, 20B, 37, or 38).

In some examples, the powering coils (e.g., vi. above or (#4) below) are used for gradient detection, as the magnetic field changes during the ramp periods of trapezoidal gradient waveforms such as those typically used in echo-planar imaging, and the changing magnetic field can be detected using the powering coils. The EMF produced at the powering coils during the ramp period is first amplified, then filtered and finally rectified to produce signals (e.g., logic signals) that act as triggering signals for the control unit. These logic signals are used to identify magnetically quiescent periods (e.g., the plateau period of the trapezoidal gradient) for substantially artifact-free operation of the electrophysiological amplifier and digitization circuit 500, 600. As no physical connection to the MRI scanner or specific pulse programming is required for the operation of the detection circuitry, this method can provide a vendor- and pulse-sequence-independent technique for accurate gradient detection. Similarly, the detection circuit also helps to identify the RF excitation zones for isolating the recording circuit from RF-induced large voltage artifacts.

In some examples, multiple systems 500, 600 can be used concurrently within the MRI bore, e.g., one for EEG and one for EKG. Each device can be programmed before use to transmit on a different, non-overlapping frequency band detectable by the MRI. This can provide frequency-division multiplexing (FDM). Additionally or alternatively, time-division multiplexing can be used. The frequency band or timeslot for each system 500, 600 can be set before placing the systems 500, 600 in the MR bore, or by download or remote control (#8 below).

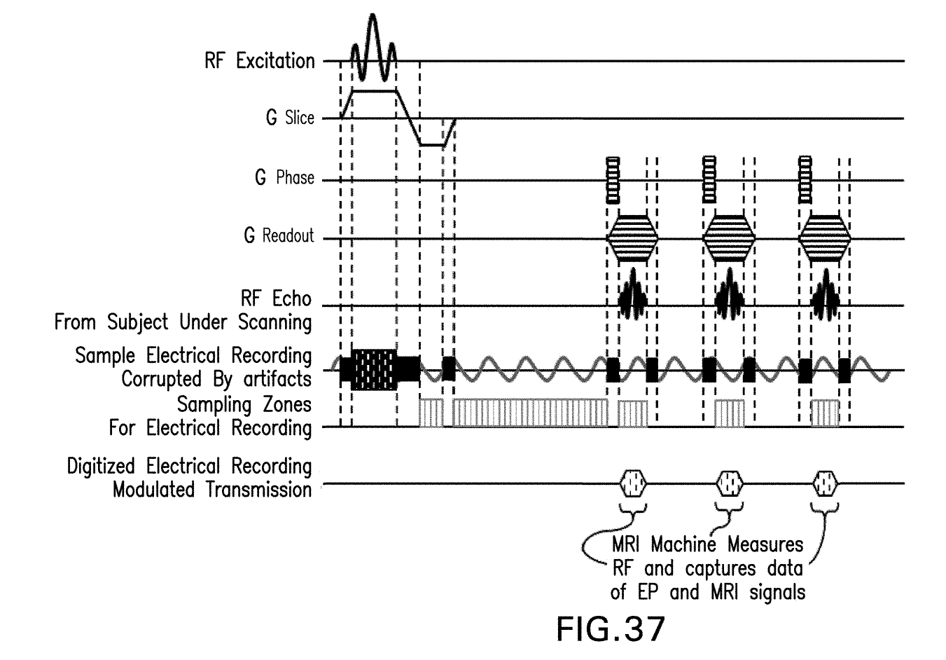

In some examples, the EP signals are transmitted back to the MRI scanner as part of the MRI image, e.g., fused with the MRI tissue image. See, e.g., FIG. 3, 10, 11, 39, 40, or 44. The resulting image can then be decomposed by into respective frequency bands for the EP and MRI images. This permits transmitting EP data without destroying MRI data. In some examples, data can be transmitted during measurement of any MRI slice, regardless of orientation. Examples are discussed herein, e.g., with reference to FIG. 37.

Many MRI machines carry out proton MRI, in which the MRI machine detects signals from H nuclei (protons). Some MRI machines support 2-channel operation, which can image protons and a different nucleus (e.g., FIG. 19) concurrently or sequentially. In some examples, the device can transmit at the proton frequency, the second-channel frequency, or both. In some examples, the MRI machine can collect MRI images on one channel and the device can communicate with the MRI machine on the other channel. This can increase the bandwidth available for transmission of EP data. Examples are discussed herein, e.g., with reference to FIG. 37. Additionally or alternatively, the MR and EP data can be time-interleaved in a single frequency band, e.g., as discussed herein with reference to FIG. 38.

In some examples, the communications unit 526 can transmit or receive data using various modulation techniques, e.g., AM or FM (e.g., frequency-shift keying 1100, FSK). Transmission can be carried out during the MRI readout phase or other phases, as noted herein. The transmit frequency can be programmed into the system(s) 500, 600 before they are used to perform measurement or stimulation, or can be provided during MR-operation (e.g., during an MR readout sequence, or between MR readout sequences). For example, the transmit frequency can be defined as an offset frequency range from a resonance frequency of interest at a given field strength. In some examples, the user of an MR scanner 532 can set the frequency of interest, then configures the system 500, 600 to transmit on that frequency. This can permit the device to operate regardless of field strength, since changes in field strength change the resonance frequency. The system 500, 600 can be programmed to match a proton frequency, carbon frequency, or other x-nucleus frequency at a given field strength. In some examples, at least one coil of the device can be a doubly-tuned coil, e.g., a coil configured to have acceptable efficiency at two different frequencies or in two different bands, e.g., for proton and carbon resonances. Such coils can be designed using conventional RF engineering techniques.

In some examples, the transmitted data are raw samples, e.g., digital values having a bit depth determined by the configuration of ADC 606. In some examples, the control unit 514, 614 can apply known compression or error-detection/-correction techniques to the data before transmission, e.g., zip or 7zip compression, or Reed-Solomon, CRC, or hash-based error-detection or -correction.

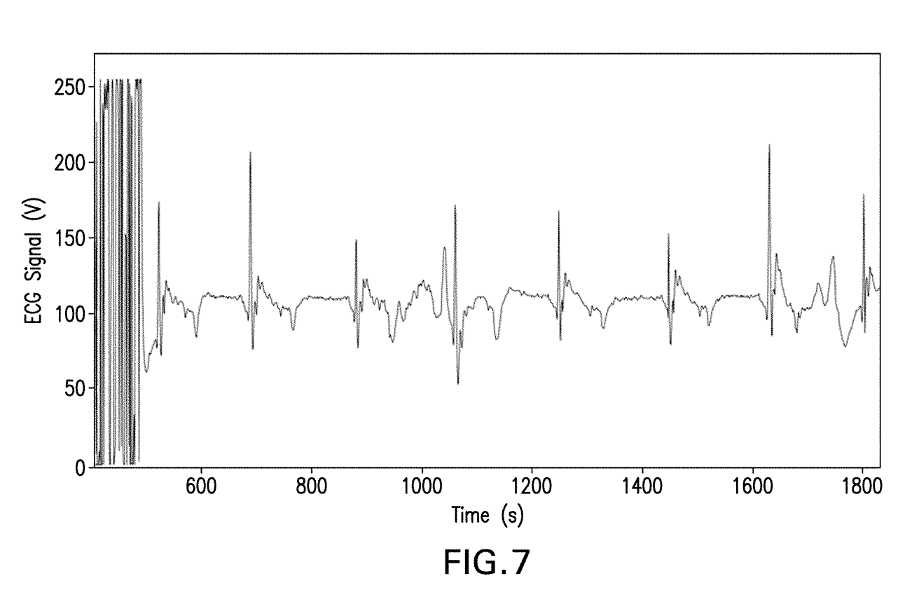

FIG. 7 shows the effect of an MR imaging sequence, in a tested example representing some prior schemes. An ECG was measured of a rat while the rat was in an MRI bore. The signal from 0 s to ˜250 s was obtained when the MRI was imaging. There was no gradient avoidance system in place for this result. As shown, in this example, the signal was entirely obscured by the MRI-induced artifacts. The signal from ˜250 s to 1800 s shows the measured rat ECG when the MRI was not imaging. As shown, the MRI gradient significantly distorts the signal of interest.

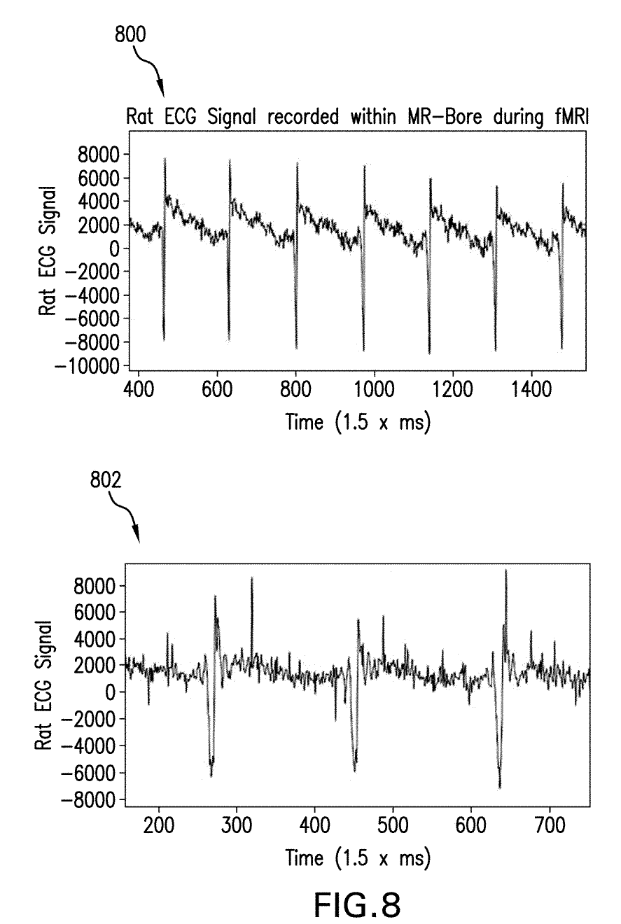

FIG. 8 shows a graphical representation of results provided using a switching circuit for gradient artifact removal. Data shown are for a rat placed inside the MR-Bore off-isocenter (plot 800) and at the isocenter (plot 802) during continuous fMRI, where each data point was synchronized with single echo data acquisition by the MRI. Note that plot 802 has a different horizontal scale than plot 800. The isocenter is the center of the MRI where magnetic fluctuation are the strongest, so artifacts may be more significant than compared to imaging off the isocenter, where magnetic strength has decayed.

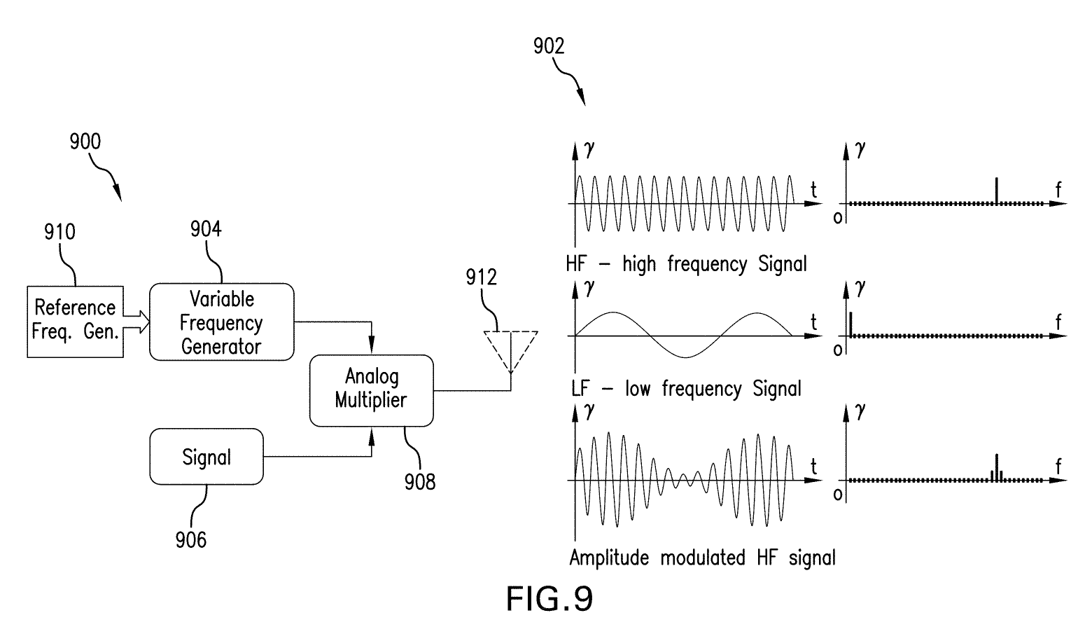

In FIG. 9, an example analog modulation system 900 and output 902 is shown for non-MR data recording utilizing MR readout-coil bandwidth not required for the reception of MR signals (e.g., echoes). The carrier frequency is matched with the RF coil of the MRI by the variable frequency generator 904. The system can receive the signal 906 to be measured, e.g., from system 500 or 600, above, and use an analog multiplier 908 or other mixer to produce the RF output signal based on signal 906. The output signal can be transmitted through an antenna 912.

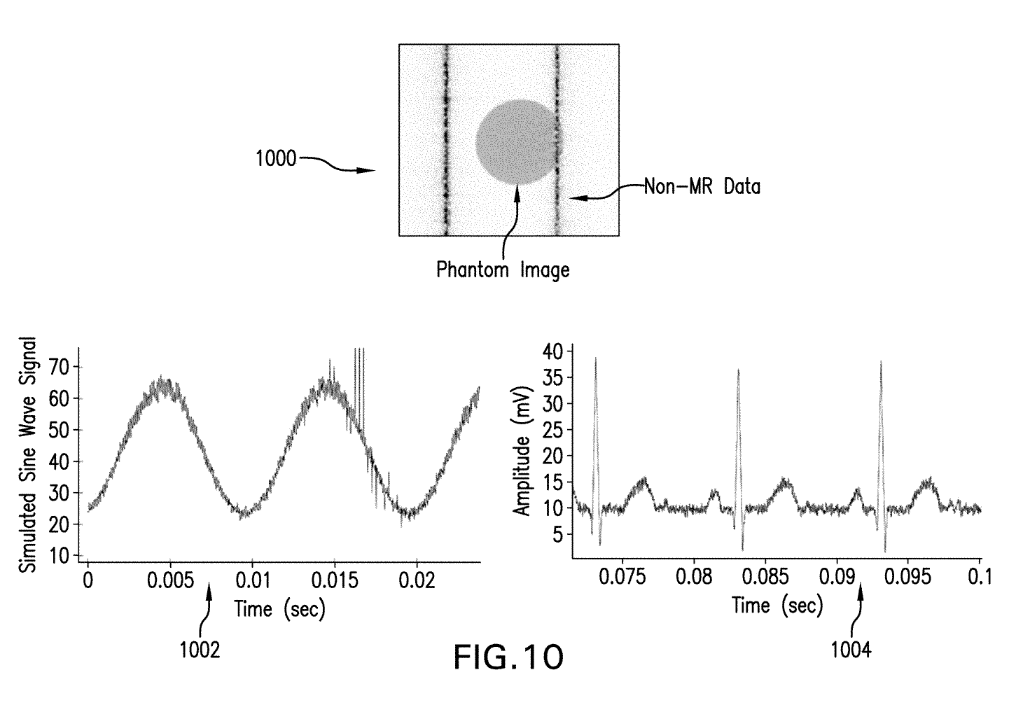

FIG. 10 shows a graphical representation of a simulated MR-image 1000 according to various examples of transmitting a non-MR signal in non-overlapping frequency bands, permitting non-MR data recording utilizing MR-bandwidth not used primarily by MR data. The transmitted signal appears as strips in the MR-image 1000. Wireless AM modulation was used in the simulation. Graph 1002 shows a simulated reconstruction of a sine wave transmitted in silico using AM modulation according to various examples herein. Graph 1004 shows a simulated reconstruction of ECG data transmitted in silico using AM modulation according to various examples herein.

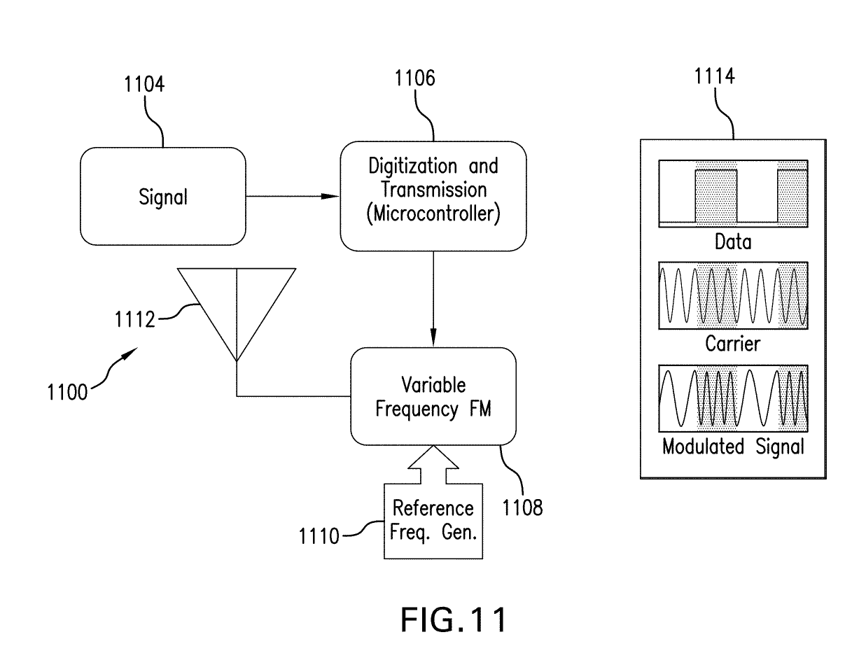

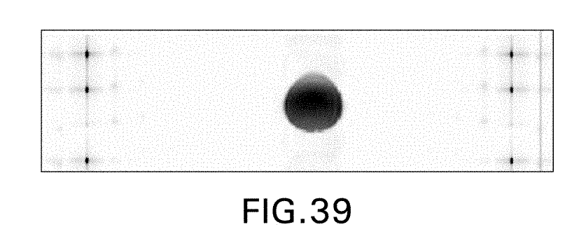

In FIG. 11, an example system 1000 for digital modulation by frequency-shift keying (FSK) is shown for non-MR data recording utilizing surplus MR-bandwidth. An example MR image is shown in FIG. 39. The example image includes non-MR data (spots near the edges of the image). The system can receive a signal 1104, e.g., from system 500 or 600. The system can include a microcontroller configured to perform digitization and transmission 1106, and a variable frequency FM generator 1108 that modulates the digitized signal and provides the signal to antenna 1112. A reference frequency generator 1110 matches the carrier frequency with the RF coil of the MRI. In some examples, the modulation technique is frequency-shift keying (FSK), e.g., as shown at the right side of FIG. 11.

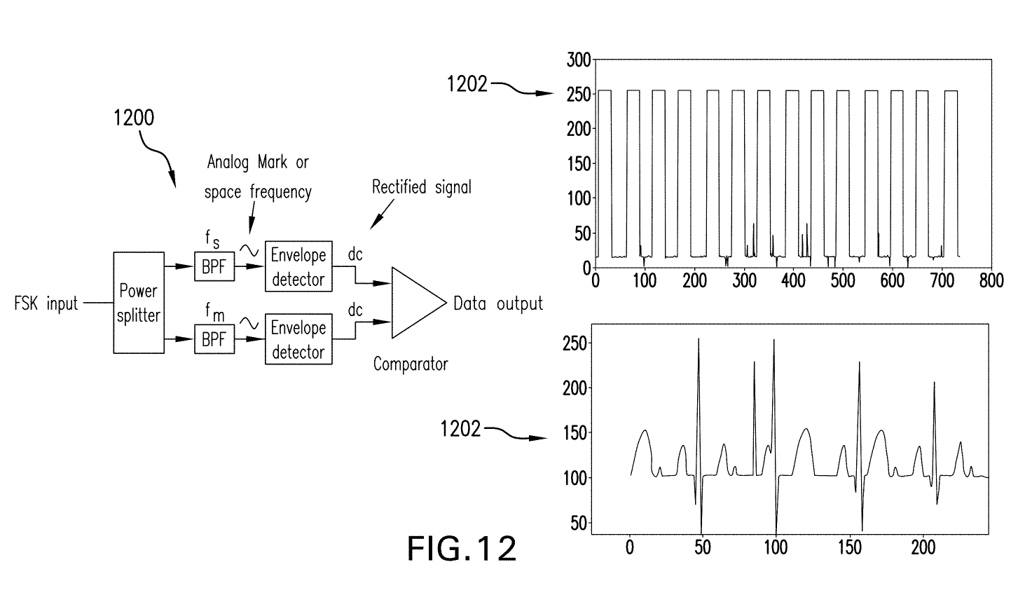

In FIG. 12, a system 1200 and a graphical output 1204 of a reconstruction of a simulated square wave 1202 and ECG signals is shown by demodulating from MRI raw data. The FSK-modulated input is split by a power splitter and provided to two bandpass filters. One passes the space frequency fₛ, and the other passes the mark frequency fₘ. Envelope detectors provide DC levels corresponding with the amount of space or mark frequency in the signal, and a comparator then provides a binary or logic value indicating whether the frequency is predominantly mark or predominantly space. Manchester, non-return-to-zero (NRZ), xb/yb (x<y, e.g., 8b/10b), or other coding schemes can be used to convert between mark/space values or sequences and 0/1 binary values.

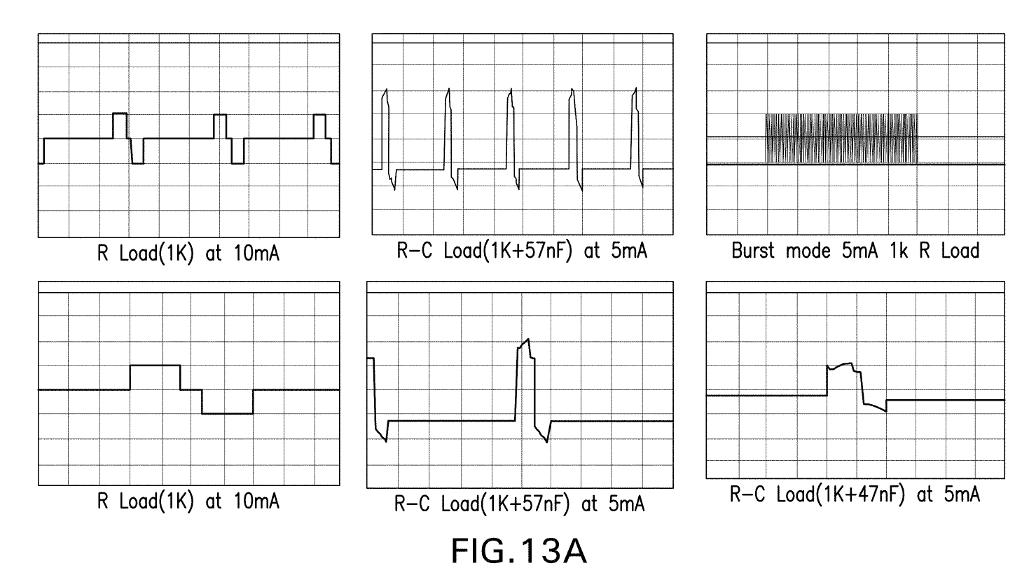

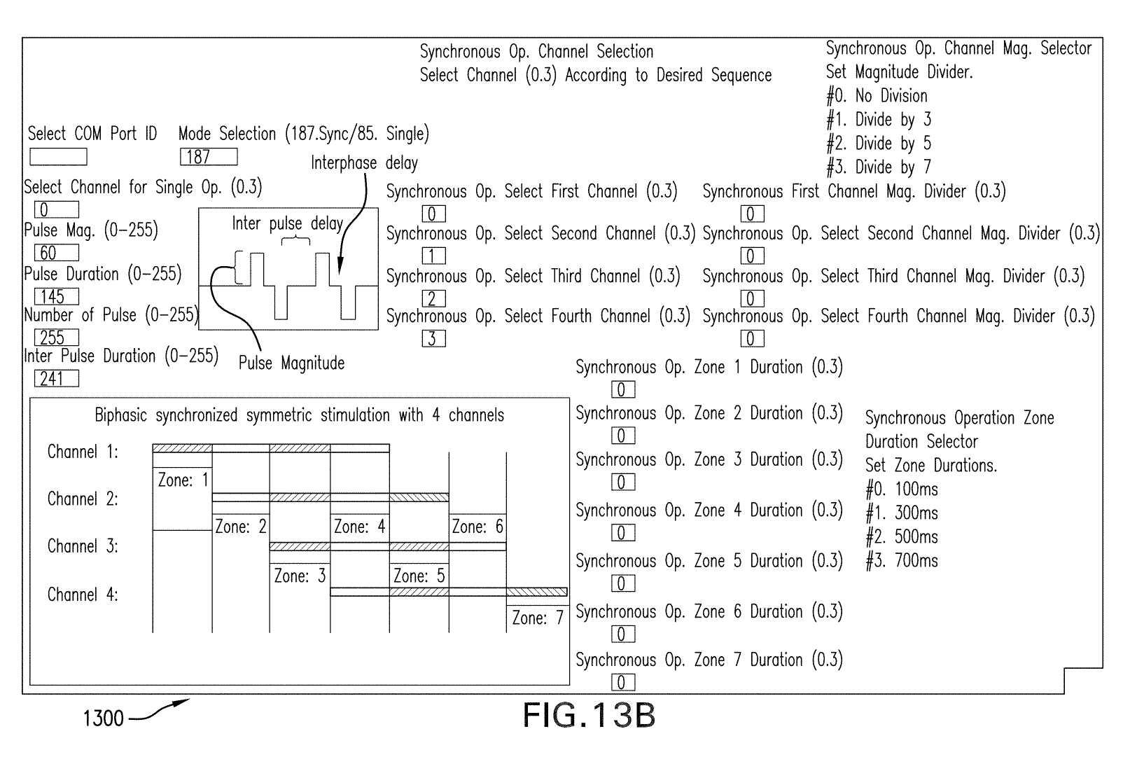

FIG. 13A shows measured data of operation of the stimulator in a tested example. The scales per div are, from left to right and top to bottom, 1 V/200 μs, 200 mV/200 μs, 500 mV (upper) and 1V (lower, dark line)/10 ms, 1V/50 μs, 200 mV/200 μs, and 500 mV/100 μs.

In FIG. 13B, a graphical representation of a LABVIEW-based GUI for control of the bi-phasic low power neuro-stimulator is shown.

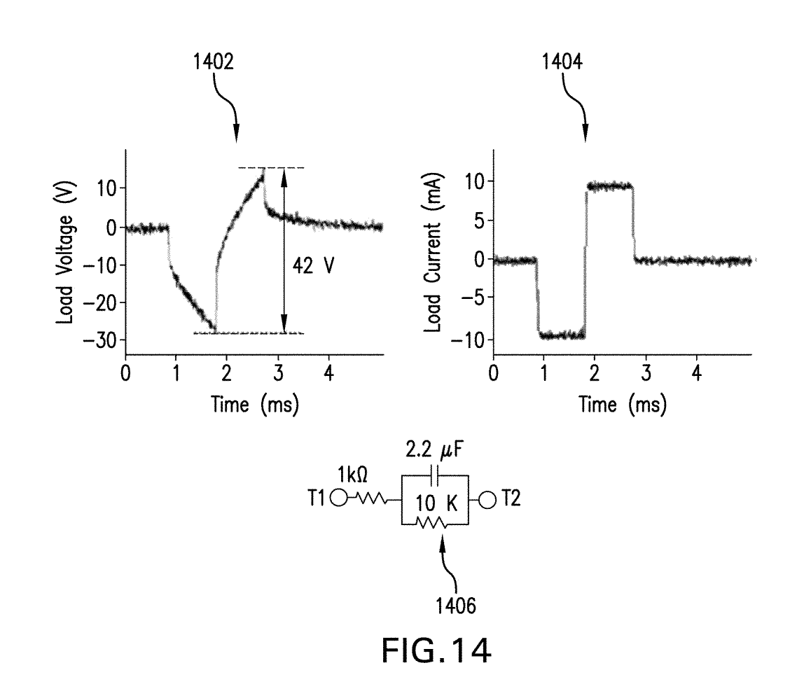

In FIG. 14, voltage and current waveforms of the bi-phasic low power neuro-stimulator are shown during stimulation across an equivalent electrode-electrolyte load impedance due to biphasic pulses, the waveforms being that of the electrode voltage 1402 and load current 1404. Additionally, an RC load representing the solution 1406 is shown.

### Illustrative Feature #4

In some examples, the Power Harvesting module 516 (FIG. 5) harvests energy for standalone operation. Illustrative Feature #4 can include components described herein with reference to Illustrative Features (i), (ii), (iv), or (vi). In some examples, a device includes at least one coil, e.g., one coil, two orthogonal coils, or three mutually orthogonal coils. Since the amount of power harvested depends on orientation (FIGS. 15-16), using multiple, orthogonal coils permits consistent power harvesting even when the magnetic-field orientation changes with respect to the device (or vice versa). Magnetic-field gradients in the MRI bore can come from any direction and have any magnitude. The coils can detect magnitude as well as direction.

FIG. 15 shows the coil of the wireless detection and powering module being placed along the encoding direction (y-axis) 1502 and along the slice selection direction (z-axis) 1506. With respect to the coil being oriented along the encoding direction 1502, the peak-peak harvesting voltage is shown to be 3.5V (plot 1504, bottom trace). The scales in plot 1504 are 1V, 2V, and 1V/div, top to bottom, and 500 μs/div. Alternatively, the oscillography of the coil being placed along the slice selection direction 1506 shows a peak-peak harvesting voltage of 5.5V (plot 1508, middle trace). The scales in plot 1508 are 1V, 2V, and 1V/div, top to bottom, and 500 μs/div.

FIG. 16 shows an example in which the coil of the wireless detection and powering module is oriented along the frequency encoding direction (x-axis) 1600. As seen by the oscilloscope plot 1602, the peak-peak harvested voltage is upwards of 40V (plot 1602, bottom trace). The scales are 10V, 2V, and 1V/div, top to bottom, and 500 μs/div.

If a device's coil is very close to tissue being imaged, the coil may have a small effect on the MR data, e.g., MRI images, collected. In some examples, therefore, the coil(s) are positioned apart from the subject 528. For example, the device can include a frame, skeleton, or other structure that retains the coils 522 (and optionally the energy-harvesting circuitry 516, e.g., rectifier(s) or regulator(s)) away from the subject 528 and the rest of the device close to the subject. This permits capturing high-quality EP signals using short electrode lead wires while still maintaining quality of the MRI scan.

In various examples using wireless powering, a pair of orthogonal coils is tuned to pick up the fast time-varying gradient fields along at least two directions for wireless power harvesting. The coils are adjustable to efficiently receive readout and phase-encoding gradients for effective power transfer. The power management module 516 can include a rectifying circuit, a DC to DC converter, and a voltage regulator, to stabilize the output voltage level and extract power out of the system. The power management module and the orthogonal coils can be placed away from the head or other body part of a subject 528 being scanned in an fMRI machine, to avoid causing any additional geometric distortion to fMRI images. In some examples, doubly tuned RF coils can be used to complement the power requirement of the system during RF transmission at an x-nucleus resonance frequency (see, e.g., FIG. 19).

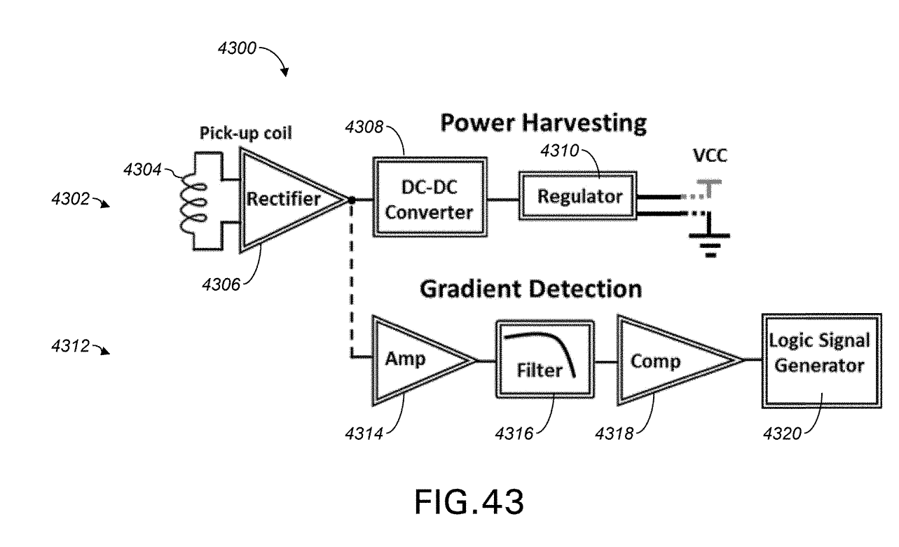

Energizing an EP-signal recording system, e.g., using powering circuitry or cables, can hinder the functionality of the MR-scanner. Conventional battery powered recording systems requires added magnetic and RF shielding, and the application of special materials is needed for the battery composition to make the system MR-safe. Accordingly, in some examples herein, the MR environment is used for wireless power harvesting for small power electronic devices. The varying magnetic field and strong RF excitation provide energy that can be harvested. The system 500, 600 can include a wireless power harvesting module 516 that extracts power from the RF excitation and also the magnetic field change due to the gradients during image acquisition. Miniaturized coils can harvest energy from MR electromagnetic fields. The harvested energy is then rectified and regulated using an IC regulator to provide the power for the recording/stimulation system (e.g., of FIG. 4, 5, or 6). Examples are discussed herein, e.g., with reference to FIG. 43.

### Illustrative Feature #5

Referring back to FIG. 5, the Stimulation Unit 512 can include a low power programmable stimulation unit such as the programmable bi-phasic current stimulator of FIG. 4. Illustrative Feature #5 can include components described herein with reference to Illustrative Features (i), (ii), (iii), (iv), or (vii).

Some examples include at least one stimulation unit 512, e.g., as discussed herein with reference to FIGS. 4, 13, and 14. The stimulator can include die-packaged analog components to reduce noise. In some examples, as little digital circuitry is used as possible. This can reduce noise due to high slew rates in digital circuitry. Details are discussed herein with reference to FIG. 4. Stimulation units can, e.g., provide at least one of electrical current, electromagnetic radiation (e.g., light, infrared, ultraviolet, or other EM), or other forms of energy to tissue of a subject. The electrical current can be used for, e.g., muscle or deep-brain stimulation. The electromagnetic radiation can be used for, e.g., optogenetic stimulation.

### Illustrative Feature #6

The Detection circuitry can include an electromagnetic receiver circuit for trigger detection, e.g., gradient detection circuitry 518. Illustrative Feature #6 can include components described herein with reference to Illustrative Features (i), (iii), (iv), or (v). Circuitry 518 can determine the magnitude and direction of a net magnetic-field gradient in the MRI bore. Circuitry 518 can be connected to the power-harvesting coils 522 to detect changes in the magnetic-field gradient based on currents flowing in those coils. In some examples, the detection circuitry detects when a gradient coil of the MRI machine turns on or off, e.g., on a trapezoidal profile. Any number >1 of coils can be used. Example coil waveforms are shown with reference to FIGS. 15, 16, 18, 37, and 38.

### Illustrative Feature #7

The Analog Processing Circuitry can include a low power, high speed switching circuit combined with a low power amplification and filtering circuit, e.g., the analog switching circuit 506 (represented in FIG. 6 by SPDT switches 618) and analog amplification and processing circuit 506. Illustrative Feature #7 can include components described herein with reference to Illustrative Features (i), (iii), or (v).

During operation, an MRI machine changes magnetic field within the bore using gradient coils. The rapidly changing magnetic field can cause transients 50-500x the EP signal amplitude. In some examples, the transients during an imaging sequence can completely obscure the EP signals to be measured. In some examples, the device includes a module 518 that detects the magnetic field changes produced by the gradient coils, e.g., automatically using a coil on the device (see #6 above). The sensing coil can be the same coil as the power-harvesting coil 522. In some examples, the magnetic-field changes are the result of, or are associated with, changes in current flows through gradient coil(s). The magnetic field can be changed, e.g., according to a trapezoidal profile of magnetic-field strength (in, e.g., mT/m) or of current (in, e.g., A) as a function of time. Herein, “activation” and “deactivation” of a gradient coil refer to ramps up or down in magnitude of a current through that gradient coil.

The switching circuit (in an example, the two SPDTs 518 in FIG. 6) can switch off the pathway through the amplifier 608 and bandpass filter 610 when a gradient coil activates or deactivates, or when the magnetic field otherwise experiences a change in magnitude or direction. The SPDTs can ground the inputs of the differential amp 608 (e.g., a differential instrumentation amplifier) and the ADC (“A2D”) 606 during such changes of any of the gradient coils. In some examples, the device is triggered by gradient readings and operates without trigger signals or other inputs from the MR scanner 532. In some examples, the circuitry automatically triggers in sync with the MR scanner 532, based on the detection of the gradients. In some examples, the circuitry automatically triggers when the magnitude of change in the magnetic field (as detected by #6) exceeds a predetermined (or downloaded, #8) threshold, e.g., of charge or of induced voltage on the readout circuitry. In a nonlimiting example, the trigger can operate the switches to ground the circuit inputs when the magnetic field changes reach a level that will induce 1V of signal at the differential amplification block input 608, or 5V at the ADC input 606. The differential amplifier 510, 608 and bandpass filtering 508, 610 can be implemented using op-amps. In some examples, trigger circuitry such as described with reference to #7 can additionally or alternatively be included in #6.

Based on a signal provided by the gradient detection circuit 518, the analog switching circuit 504, 618 will disconnect or reconnect the inputs to the recording circuit 506, 508, 608, 610 to maintain the series of analog stages in a non-saturated condition. When disconnected, those inputs can be latched together to the system ground, resulting in no output signal from the differential amplifier 506, 608 (e.g., a first stage in the analog recording circuit). The analog switching circuit 504, 618 can include CMOS single pole double throw (SPDT) switch IC(s) to connect the recording leads to the input of the recording circuit. In an example discrete component design, the switch only supports binary functionality (step response profile). In an example ASIC design, the switch can be configured to emulate different profiles during switching. These can include a ramping, spiral, and exponential switching profiles. The basis of using a different switching profile than the conventional step response profile is to reduce the switching noise which is injected into the recording system. In some examples, the switching circuitry 504, 618 can additionally ground the inputs of ADC 510, 606 during transients, to maintain the input circuitry of the ADC 510, 606 in a non-saturated condition.

Like the analog switching circuitry 504, 618, the gain switching circuitry 506, 608 can uses signals provided by the gradient detection circuitry 518. Gain switching can further reduce the RF and gradient induced noise. The gain of the amplifiers 506, 608 can be altered to provide attenuation when the RF or gradient pulse is present and to provide amplification when the pulses are not present. An example discrete component design uses a SPDT CMOS switch in order to achieve a quick transition between resistors to alter the gain. An example ASIC design manages the gain using a VGA (Variable Gain amplifier) or a PGA (Programmable Gain Amplifier), e.g., based on a transconductance amplifier.

A control unit (e.g., a microcontroller) 514, 614 and analog to digital converter (ADC) 510, 606 can be used to sample the analog signal based on signals provided by the gradient detection circuit 518. The ADC can include a low-power, triggered converter for digitization of analog signals. The ADC can be triggered to sample in the interval of the imaging sequence when RF and gradient pulses are not present. This reduces artifacts in the digitized signal.

In some examples, in addition to sampling between RF and gradient pulses, the microcontroller can be configured to monitor the timing intervals of the RF and the gradient pulses to predict their occurrence, and to take predetermined actions to avoid the induced noise. The microcontroller can take these predetermined actions with the help of modules such as timers and interrupt generators. In some examples, the shape and time interval of gradient and RF pulses will not change during an MR cycle, since those parameters determine the type of image which is obtained from the MRI. Therefore, the control unit 514, 614 can forecast the timing intervals of the RF and gradient pulses based on measurements. In the event that future pulses do not match the forecast, the control unit can respond to the pulses and update the forecast, as discussed below.

In some examples, the control unit 514, 614, e.g., a microcontroller, can be programmed to interrupt on the edges of a binary output from the gradient detection circuit 518, 612. The interrupt can suspend normal code execution and begin the execution of a function specified in the interrupt vector table. This interrupt function can determine sampling times and control the analog processing circuits (e.g., blocks 506, 508, 608, or 610). In response to the edge interrupt, the microcontroller's timer can be configured to store the timer count in a variable and then take the difference between the previous timer value and the new one. This give the microcontroller the timer count between edges of the gradient detection output. Using this information, the microcontroller can determine how many samples to acquire (e.g., given a predetermined, substantially constant sampling time or rate), and when to conduct the analog and gain switching.

In some examples, an adaptive-sampling trigger determined, e.g., using edge timers, is assigned a lower priority than the trigger provided by the gradient detection circuit. For example, if there is a change in the imaging sequence that causes the gradient to arrive before anticipated, the microcontroller can cease recording or stimulation; update timers or counters; or take the actions described previously. In some examples, the microcontroller monitors the time spent recording. If the gradient timings change, the microcontroller will adjust its reference time to accommodate recording to the new imaging sequence.

In some examples, before a pulse sequence during which MR data is collected, the control system 534 operates the MR-scanner 532 for a number of pulses or pulse sequences during which MR data is not collected. Those pulses or sequences can be referred to as “dummy” pulses or sequences. In some examples, the microcontroller measures gradient edges and timings, e.g., as discussed above, during dummy pulses or sequences. The microcontroller can then determine the length of a pulse sequence, e.g., as a shift value for which the autocorrelation of the measured signals is highest for the tested shift values, or is above a predetermined threshold. Additionally or alternatively, the microcontroller can determine when the pulse sequence starts, e.g., by finding the longest delay time between two consecutive pulses and assuming that the latter of those two pulses is the beginning of a pulse sequence.

In some examples, the microcontroller is pre-programmed with information regarding the timing of a pulse sequence. The microcontroller can then determine a current point in the pulse sequence, by comparing observed gradient intervals to those in the pre-programmed information. For example, the pre-programmed information can include text representing times between gradients (quantized appropriately), and the microcontroller can use text-search algorithms such as KMP to search the text.

### Illustrative Feature #8

In some examples, the device can be programmed to detect specific pulse sequences that convey data, e.g., to download data to the device, such as stimulation sequences or parameters, or to control the device remotely, e.g., to enable and disable the device or to set the transmission frequency. This can permit interacting with the device in the MR bore, e.g., for remote control or information download, without a requirement for another transceiver or for a wired control connection. Control information can be conveyed by the MRI machine, e.g., using the readout coil or one or more gradient coils. Information can be conveyed by the sequence of pulses from the coils, the duration of pulses, the spacing between pulses, which coils are used (e.g., the gradient direction), or any combination thereof. Known modulation, compression, error-detection, or error-correction techniques can be used when transmitting data. For example, self-clocking encodings such as NRZ or Manchester coding can be used. The device can decode the control signal from changes in the magnetic field around the device, e.g., by demodulating or otherwise reversing the modulation or compression techniques used. The device can carry out two-way communications with the MRI machine via transmissions, e.g., at a readout frequency. Illustrative Feature #8 can include components described herein with reference to Illustrative Features (i), (iii), (iv), or (v).

### Illustrative Feature #9

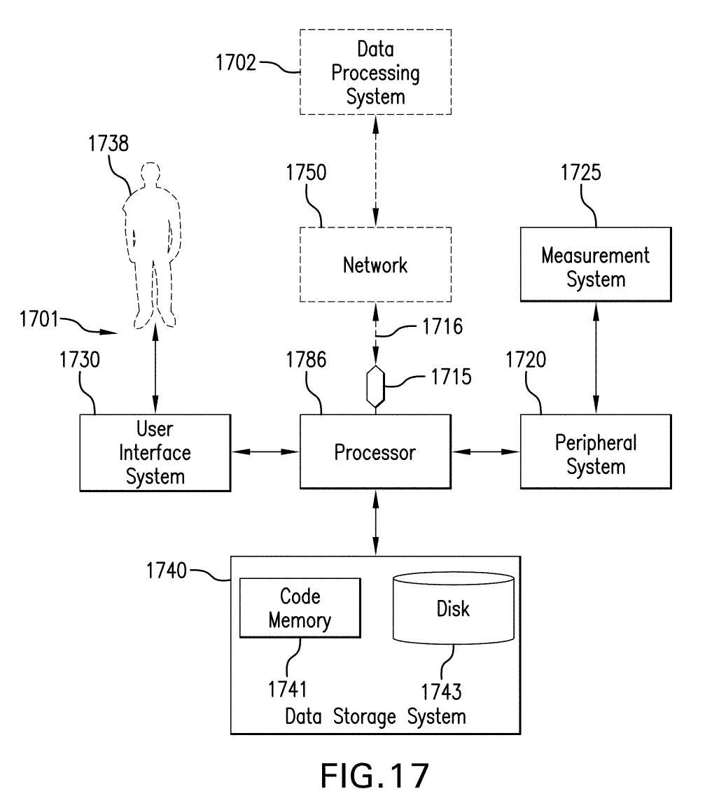

Arbitrary-pattern generation can include, in some examples, instances where neuromodulation circuitry driving a stimulator can be programmed, e.g., through software or firmware, to generate arbitrary patterns. Examples of such circuitry or other programmable devices as discussed herein with reference to FIGS. 4 and 17. The magnitude, frequency, sequence and other parameters can be adjusted by the user to create waveforms specific to desired application. In some examples, an M-sequence can be encoded within the stimulation waveform. In some examples, MRI controller 534 can additionally control a stimulator 512 (FIG. 4). Illustrative Feature #9 can include components described herein with reference to Illustrative Features (i), (iii), (iv), or (v).

### Further Illustrative Feature Combinations

Some examples include components of each of Illustrative Features #1-#4, plus components of at least one block selected from Illustrative Features #5-#9.

Some examples include components of at least one of, or each of, Illustrative Features (i)-(vii). Some examples include components of each of Illustrative Features (i), (iii), and (v).

Some examples include at least one of the following features, labeled A-T.

A. MR-Compatible recording and stimulation system that utilizes MR hardware capabilities to generate, transmit non-MR signals in synchronization with standard MR scanner.

B. A method for utilizing analog circuitry for effective capturing of non-MR signals within MR scanner and minimize gradient and RF artifacts.

C. A synchronized and wirelessly controlled stimulation platform for different stimulation modalities (current stimulation, optical stimulation, magnetic stimulation etc.).

D. A method for utilizing electromagnetic field within MR scanner for detecting and transmitting non-MR data and receiving using present MR hardware as described in A.

E. A modulation and demodulation scheme for high speed acquisition and transmission of non-MR data as described in D.

F. A method for harvesting power within an MR bore, utilizing the EM field within MR apparatus and, optionally, additional environment energy scavenging.

G. A MRI sequence to incorporate fast unidirectional or bi-directional communication protocol as described in E, combined with energy harvesting as described in F.

H. A method for harvesting power as described in F and utilization of doubly tuned coil, designed specifically for fast bi-directional telemetry as stated in E to accommodate multiple channel recording of electrophysiological signal.

I. An MR-Compatible recording and stimulation system that utilizes MR hardware capabilities to generate, transmit non-MR signals in synchronization with standard MR scanner.

J. A method for utilizing analog circuitry for effective capturing of non-MR signals within MR scanner and minimize gradient and RF artifacts.

K. A synchronized and wirelessly controlled stimulation platform for different stimulation modalities (current stimulation, optical stimulation, magnetic stimulation etc.).

L. A method for utilizing electromagnetic field within MR scanner for detecting and transmitting non-MR data and receiving using present MR hardware as described in I.

M. A modulation and demodulation scheme for high speed acquisition and transmission of non-MR data as described in L.

N. A method for harvesting power within MR bore utilizing EM field within MR apparatus and additional environment energy scavenging.

O. A MRI sequence to incorporate fast communication protocol as described in M and energy harvesting as described in N.

P. A high-voltage-compliant stimulation system that combines with MR system and provides stimulation synchronized with MR image acquisition.

Q. An interface system for communication of control parameters between device within MR bore and user.

R. An integrated software system that works as add-on to a conventional MRI GUI to completely control stimulation and recording system within MR apparatus.

S. An integrated system as described in R, capable of continuous processing and display of non-MR data alongside MR acquired images.

T. Combinations of at least one of A-S.

Steps of various methods described herein can be performed in any order except when otherwise specified, or when data from an earlier step is used in a later step. Example method(s) described herein are not limited to being carried out by components particularly identified in discussions of those methods.

### Illustrative Operations

In some examples, system characterization and MR-Compatibility testing can be carried out, e.g., on healthy rat subjects within a 7 T MR scanner (Bruker, USA, MA). Examples include evaluation of mutual interference between fMRI and EEG recording/stimulation system and also SAR and monitoring of temperature increase on phantoms as well as animal subjects. RF device safety (SAR) can be carried out using FDTD analysis before animal experiments. Moreover, the effect of imaging pulses and gradient magnetic field on the recorded electrophysiological signal can be analyzed for firstly the larger ECG signals and later for smaller EEG, ECoG signals. Applicability of the proposed design can be tested on small animal subjects (e.g., rats) for cross-correlation between (1) large-scale fMRI and EEG recording and local neural recording, (2) large-scale fMRI and local stimulation.

An example system discussed herein, and methods discussed herein, were tested using a BRUKER 7T animal MRI. The subjects in these experiments were of the species Rattus norvegicus (rat). The experiments conducted included monitoring the rat's EKG and evoked potentials. A system including components described herein with reference to systems 500, 600), including the gradient and/or power-harvesting coils 522, can be placed inside the bore, adjacent to the subject. The electrodes for EKG, EEG, LFP, etc. can be securely fixed at the site of the signal source. The electrodes can be properly oriented as needed for the specific type of signal to be captured. The leads from the electrode can be arranged as straight as possible as they connect with the recording device. The subject 528 and the system can be positioned inside the MRI bore. The subject can then be imaged using any type of gradient echo sequence to power and activate the device. The EP measurement system can operate as explained previously and transmit the non-MR data to be reconstructed as discussed herein. The EP data can then be visualized alongside the MRI image for the user's convenience. In some examples, the EP measurement system will automatically power off after each MR sequence, e.g., based on an elapsed time since the last magnetic-field change or RF pulse.

### Illustrative Data-Processing Components and Features

FIG. 17 is a high-level diagram showing the components of an example data-processing system 1701 for capturing or analyzing data and performing other functions described herein, and related components. System 1701 can include or communicate with a measurement system 1725, e.g., system 500 or 600 described herein. System 1701 can include components or carry out functions identified above with reference to labels (i)-(vii), #1-#8, or A-T. The illustrated system 1701 includes a processor 1786, a peripheral system 1720, a user interface system 1730, and a data storage system 1740. The peripheral system 1720, the user interface system 1730, and the data storage system 1740 are communicatively connected to the processor 1786. Processor 1786 can be communicatively connected to network 1750 (shown in phantom), e.g., the Internet or a leased line, as discussed below. Devices shown in FIG. 4, 5, 6, 9, 11, 12, 19, 20A, 20B, 22A, 22B, or 43 can each include or connect with one or more of systems 1786, 1720, 1730, 1740, and can each connect to one or more network(s) 1750. Processor 1786, and other processing devices described herein, can each include one or more microprocessors, microcontrollers, field-programmable gate arrays (FPGAs), application-specific integrated circuits (ASICs), programmable logic devices (PLDs), programmable logic arrays (PLAs), programmable array logic devices (PALs), or digital signal processors (DSPs). In some examples, system 1701 omits user interface system 1730. In some examples, system 1701 includes at least one of components 1720, 1730, 1740, or 1786. Components of system 1701 can be die-packaged as described above, or otherwise packaged in or using MR-Safe or MR-Compatible materials or structures. Components of system 1701 can be implemented using analog, digital, or mixed-signal components.

Processor 1786 can implement processes of various aspects described herein. Processor 1786 and related components can, e.g., carry out processes for measuring EP signals and transmitting those signals in synchronization with MRI operations. Processor 1786 can be implemented using analog, digital, or mixed-signal components.

Processor 1786 can be or include one or more device(s) for automatically operating on data, e.g., a central processing unit (CPU), MCU, desktop computer, laptop computer, mainframe computer, personal digital assistant, digital camera, cellular phone, smartphone, or any other device for processing data, managing data, or handling data, whether implemented with electrical, magnetic, optical, biological components, or otherwise.

The phrase “communicatively connected” includes any type of connection, wired or wireless, for communicating data between devices or processors. These devices or processors can be located in physical proximity or not. For example, subsystems such as peripheral system 1720, user interface system 1730, and data storage system 1740 are shown separately from the processor 1786 but can be stored completely or partially within the processor 1786.

The peripheral system 1720 can include or be communicatively connected with one or more devices configured or otherwise adapted to provide digital content records to the processor 1786 or to take action in response to processor 186. For example, the peripheral system 1720 can include digital still cameras, digital video cameras, cellular phones, or other data processors. The processor 1786, upon receipt of digital content records from a device in the peripheral system 1720, can store such digital content records in the data storage system 1740.

The user interface system 1730 can convey information in either direction, or in both directions, between a user 1738 and the processor 1786 or other components of system 1701. The user interface system 1730 can present interfaces shown in FIGS. 13A and 13B. The user interface system 1730 can include a mouse, a keyboard, another computer (connected, e.g., via a network or a null-modem cable), or any device or combination of devices from which data is input to the processor 1786. The user interface system 1730 also can include a display device, a processor-accessible memory, or any device or combination of devices to which data is output by the processor 1786. The user interface system 1730 and the data storage system 1740 can share a processor-accessible memory.

In various aspects, processor 1786 includes or is connected to communication interface 1715 that is coupled via network link 1716 (shown in phantom) to network 1750. For example, communication interface 1715 can include an integrated services digital network (ISDN) terminal adapter or a modem to communicate data via a telephone line; a network interface to communicate data via a local-area network (LAN), e.g., an Ethernet LAN, or wide-area network (WAN); or a radio to communicate data via a wireless link, e.g., WIFI or GSM. Communication interface 1715 sends and receives electrical, electromagnetic, or optical signals that carry digital or analog data streams representing various types of information across network link 1716 to network 1750. Network link 1716 can be connected to network 1750 via a switch, gateway, hub, router, or other networking device.

In various aspects, system 1701 can communicate, e.g., via network 1750, with a data processing system 1702, which can include the same types of components as system 1701 but is not required to be identical thereto. Systems 1701, 1702 can be communicatively connected via the network 1750. Each system 1701, 1702 can execute computer program instructions to measure or transmit measurements, as described herein.

Processor 1786 can send messages and receive data, including program code, through network 1750, network link 1716, and communication interface 1715. For example, a server can store requested code for an application program (e.g., a JAVA applet) on a tangible non-volatile computer-readable storage medium to which it is connected. The server can retrieve the code from the medium and transmit it through network 1750 to communication interface 1715. The received code can be executed by processor 1786 as it is received, or stored in data storage system 1740 for later execution.

Data storage system 1740 can include or be communicatively connected with one or more processor-accessible memories configured or otherwise adapted to store information. The memories can be, e.g., within a chassis or as parts of a distributed system. The phrase “processor-accessible memory” is intended to include any data storage device to or from which processor 1786 can transfer data (using appropriate components of peripheral system 1720), whether volatile or nonvolatile; removable or fixed; electronic, magnetic, optical, chemical, mechanical, or otherwise. Example processor-accessible memories include but are not limited to: registers, floppy disks, hard disks, tapes, bar codes, Compact Discs, DVDs, read-only memories (ROM), erasable programmable read-only memories (EPROM, EEPROM, or Flash), and random-access memories (RAMs). One of the processor-accessible memories in the data storage system 1740 can be a tangible non-transitory computer-readable storage medium, i.e., a non-transitory device or article of manufacture that participates in storing instructions that can be provided to processor 1786 for execution.

In an example, data storage system 1740 includes code memory 1741, e.g., a RAM, and disk 1743, e.g., a tangible computer-readable rotational storage device or medium such as a hard drive. Computer program instructions are read into code memory 1741 from disk 1743. Processor 1786 then executes one or more sequences of the computer program instructions loaded into code memory 1741, as a result performing process steps described herein. In this way, processor 1786 carries out a computer implemented process. For example, steps of methods described herein, blocks of the flowchart illustrations or block diagrams herein, and combinations of those, can be implemented by computer program instructions. Code memory 1741 can also store data, or can store only code.

In the illustrated example, systems 1701 or 1702 can be computing nodes in a cluster computing system, e.g., a cloud service or other cluster system (“computing cluster” or “cluster”) having several discrete computing nodes (systems 1701, 1702) that work together to accomplish a computing task assigned to the cluster as a whole. In some examples, at least one of systems 1701, 1702 can be a client of a cluster and can submit jobs to the cluster and/or receive job results from the cluster. Nodes in the cluster can, e.g., share resources, balance load, increase performance, and/or provide fail-over support and/or redundancy. Additionally or alternatively, at least one of systems 1701, 1702 can communicate with the cluster, e.g., with a load-balancing or job-coordination device of the cluster, and the cluster or components thereof can route transmissions to individual nodes.

Some cluster-based systems can have all or a portion of the cluster deployed in the cloud. Cloud computing allows for computing resources to be provided as services rather than a deliverable product. For example, in a cloud-computing environment, resources such as computing power, software, information, and/or network connectivity are provided (for example, through a rental agreement) over a network, such as the Internet. As used herein, the term “computing” used with reference to computing clusters, nodes, and jobs refers generally to computation, data manipulation, and/or other programmatically-controlled operations. The term “resource” used with reference to clusters, nodes, and jobs refers generally to any commodity and/or service provided by the cluster for use by jobs. Resources can include processor cycles, disk space, RAM space, network bandwidth (uplink, downlink, or both), prioritized network channels such as those used for communications with quality-of-service (QoS) guarantees, backup tape space and/or mounting/unmounting services, electrical power, etc.

Network 1750 can represent wireless communications via MRI frequencies, e.g., as discussed herein with reference to FIGS. 5, 6, and 9-12. System 1701 can represent a device as described herein, and system 1702 can represent an MRI machine.

Furthermore, various aspects herein may be embodied as computer program products including computer readable program code (“program code”) stored on a computer readable medium, e.g., a tangible non-transitory computer storage medium or a communication medium. A computer storage medium can include tangible storage units such as volatile memory, nonvolatile memory, or other persistent or auxiliary computer storage media, removable and non-removable computer storage media implemented in any method or technology for storage of information such as computer-readable instructions, data structures, program modules, or other data. A computer storage medium can be manufactured as is conventional for such articles, e.g., by pressing a CD-ROM or electronically writing data into a Flash memory. In contrast to computer storage media, communication media may embody computer-readable instructions, data structures, program modules, or other data in a modulated data signal, such as a carrier wave or other transmission mechanism. As defined herein, computer storage media do not include communication media. That is, computer storage media do not include communications media consisting solely of a modulated data signal, a carrier wave, or a propagated signal, per se.

The program code includes computer program instructions that can be loaded into processor 1786 (and possibly also other processors), and that, when loaded into processor 1786, cause functions, acts, or operational steps of various aspects herein to be performed by processor 1786 (or other processor). Computer program code for carrying out operations for various aspects described herein may be written in any combination of one or more programming language(s), and can be loaded from disk 1743 into code memory 1741 for execution. The program code may execute, e.g., entirely on processor 1786, partly on processor 1786 and partly on a remote computer connected to network 1750, or entirely on the remote computer.

In some examples, processor 1786 or other components shown in FIG. 17 can be communicatively connected with EP sensors such as those shown in FIG. 19, 22A, or 22B. In some examples, processor 1786 can be configured to carry out operations, e.g., signal processing, illustrated in FIG. 19-21 or 23-33.

### Further Illustrative Operations and Configurations

FIG. 18 is a pulse-sequence diagram of an example MRI and EP readout sequence according to some examples. Throughout this discussion, including FIGS. 18, 37, and 38, illustrated pulse sequences are nonlimiting examples. The illustrated pulse sequences, or other pulse sequences, can be adapted according to the type of MR data to be collected or the conditions under which that data should be collected. In FIGS. 18, 37, and 38, “gradients” represent magnetic-field gradient magnitude, or current through gradient coil(s). Ramps on the gradients correspond to changes in the magnetic field. Hatched hexagons represent periods during which the magnetic field changes repeatedly, rapidly, continuously, or continually.

As shown, the MRI machine applies an RF pulse concurrently with a slice gradient. The MRI machine later applies a phase gradient, and still later applies a frequency/readout (“freq/read”) gradient to measure the echo (e.g., a gradient echo or Hahn echo) from the resonating nuclei (e.g., protons, ¹H). EP signal measurement can be carried out, for example, at times such as those represented by the “MSMT” (e.g., Multi Switch Multi Throw) boxes, e.g., after cessation of a change in the magnetic field. Detection circuitry (#1, #2, #7) that performs EP signal measurement can be isolated (#6, #7) from transients during magnetic-field changes, as represented by the black boxes on the “EP signal measurement” line. EP signals can be measured any time except during the black-boxed magnetic-field changes, in some examples, e.g., at a time other than during magnetic-field changes. In some examples, stimulation (#5) can be carried out any time, or any time except during magnetic field changes (indicated by black boxes). In some examples, stimulations units (#5) can be isolated (#6, #7) from transients during magnetic field changes, as represented by the black boxes on the “EP signal measurement” line.

As used herein, periods of “quiescent” magnetic field refer to times other than during magnetic-field changes. During a quiescent period, a static magnetic field or gradient may be present, or it may not. The term “other than” does not imply or require that, during a time other than during magnetic-field changes, the magnetic field around the device be absolutely or mathematically constant. However, during a time other than during magnetic-field changes (e.g., during a quiescent period), artifacts due to magnetic-field changes can have a magnitude that is, e.g., below the noise floor of an EP detection or stimulation unit; below a predetermined percentage of a peak-to-peak signal voltage of an EP detection unit (e.g., <20%, <10%, <5%, or <1%); or below a predetermined slew rate in dV/dt (V/s). Additionally or alternatively, during a time other than during magnetic-field changes, the magnetic field can be changing at a rate below a predetermined dB/dt (T/s) value.

In the illustrated example, quiescent period 1804 commences with the end of the rising edge of the “freq/read” (frequency/readout) gradient. Quiescent period 1804 terminates with the beginning of the falling edge of the freq/read gradient.

In some examples, the system can detect activity periods of the MRI coil(s) of the MRI scanner, e.g., during the slice, phase, or frequency/readout gradient trapezoidal pulses (#6). The system can then transmit data corresponding to the electrophysiological signal (#3) during the activity period. This is depicted graphically by the hexagons on the “data transmission” line. With reference to FIG. 18, the first two activity periods 1802 shown do not correspond to MRI readout, so the MRI machine may ignore the transmissions during periods 1802. The third activity period 1804 does correspond to MRI readout, so the MRI machine will capture the transmitted data during period 1804 (e.g., FIGS. 3, 5, 6, and 9-12). The device may be configured to detect which activity period is the readout period and only transmit during the readout period 1804, but that is not required. Additionally or alternatively, the device can transmit during period(s) 1802.

In some examples, the pulse sequence can be preceded or triggered by signals sent from the system 500, 600 to the MR control system 534 via receive coils of MR scanner 532. Examples are discussed herein, e.g., with reference to FIG. 37.

FIG. 19 shows data and example EEG devices. In FIGS. 19, 22A, 22B, a graphical depiction of a brain represents a biological system from which EP data are being collected. This can be a brain, a heart, or another organ of, e.g., a human or animal subject, in various nonlimiting examples. Similarly, although FIGS. 19, 22A, and 22B, and other examples herein, are discussed with reference to EEG data, various techniques can components described herein can additionally or alternatively be used for measuring other types of EP data, e.g., ECG data.

In FIG. 19, the right-hand side shows an example multi-layer EEG cap design 1900 for unipolar and bi-polar recording. The multi-layer EEG cap design comprises a reference electrode 1902, bipolar electrodes 1904, conductive gel inputs 1906, device 1908 (e.g., an amplifier or reference unit, or portion thereof, as described below), and an insulating layer 1910. Further examples are shown in FIG. 22A. In some examples, respective signals from electrodes 1904 in an adjacent pair of + and − electrodes can be fed to an amplifier for bipolar recording. In some examples of unipolar recording, signals from a + or −, or both a + and a −, can be fed to an amplifier as an active signal. A reference signal can be provided, e.g., as discussed herein with reference to FIGS. 22A and 22B. In some examples, recording electrodes 1904 can be spread across the skull.

In FIG. 19, concurrent fMRI and EEG is shown for utilizing MR-receive coil surplus bandwidth to acquire multi-channel EEG signals. The right side shows a cap 1900 having a plurality of bipolar electrodes 1904, and a layer of reference electrodes passing through the layer of bipolar electrodes 1904. See also FIG. 22B, right side. In some examples, the amplifier can select whether to use the local bipolar electrodes for bipolar recording, or whether to select a reference signal from elsewhere in the cap for unipolar recording, e.g., in response to an operator request.

On the left side are shown examples of MR acquisition. The illustrated example represents an MRI system using a double-tuned RF receiver coil, which can permit detecting resonance data from both protons (′H) and other nuclei (“x-nucleus”). In other examples, signals can be detected in a common band, e.g., with the ¹H band. In some examples, a double-tuned RF receiver coil is not used. Detection of EP data, e.g., of EP signals from system 500, and MR data in the same band is referred to herein as “Extended FOV.”

FIGS. 20A and 20B show example circuits for wireless EEG recording and gradient detection. In FIG. 20A, compared to FIG. 5, the amplifier 2006 has an additional input from MCU 2012 to control the gain. This permits adjusting the gain to avoid or mitigate MRI artifacts. At the bottom of FIG. 20B, the gain is high when the switching trigger is on, and low when the switching trigger is off.

The circuit in FIG. 20A can include recording/stimulating electrodes 2002 (which can represent electrodes 502), a switching circuit 2004 (which can represent 504), a variable gain amplifier 2006 (which can represent 506), an analog filter 2008 (which can represent 508), an analog to digital converter (ADC) 2010 (which can represent 510), a microcontroller 2012 (which can represent 514), a wireless power harvesting module 2014 (which can represent 516), a gradient and RF pulse detector 2016 (which can represent 518), a transmitting module 2018 (which can represent 520), a wireless power harvesting antenna 2020 (which can represent 522), and a data transmitting antenna 2022 (which can represent 524).

Some techniques described above disconnect the amplifier input during transients, e.g., as discussed herein with reference to FIGS. 5 and 6 and Illustrative Features (iii) and #7 above. Some examples use additional circuitry to further reduce transients. In some examples, a variable-gain circuit is used to switch off inputs and reduce input gain during switching, as discussed herein with reference to FIGS. 5 and 6.

Some examples relate to analog switching. The EEG signals pass through local on-board analog processing and switching circuitry before digitization and wireless transmission. Logic signals from the gradient detection circuit (#6 above) can be used for synchronized activation and deactivation of the rapid-responding (e.g., <200 ns) Single-Pole-Double-Throw (SPDT) analog switching circuits 504, 618, 2004 to isolate analog channels in presence of gradient and RF artifacts.

In some examples, the analog-to-digital converter 2010 uses synchronized sampling, e.g., as in Illustrative Features (v) and #3. In some examples, a low-power (e.g., <0.1 mW) high-resolution (e.g., 16-bit, 0.5 μV) analog to digital converter 2010 is used to digitize the analog signal during time periods that are substantially electromagnetically quiescent, e.g., in which the magnetic gradients are not changing. Logic signals from gradient (#6) and RF detection circuits enable the implementation of adaptive sampling methods, since gradient changes are precisely identified. An ultra-low power microcontroller 514, 614, 2012 can be used to control the digitization circuit and to synchronize transmission to the MRI receive coil. Some examples include a 16-bit low-power ADC within the measurement system, or >16 bits of ADC resolution.

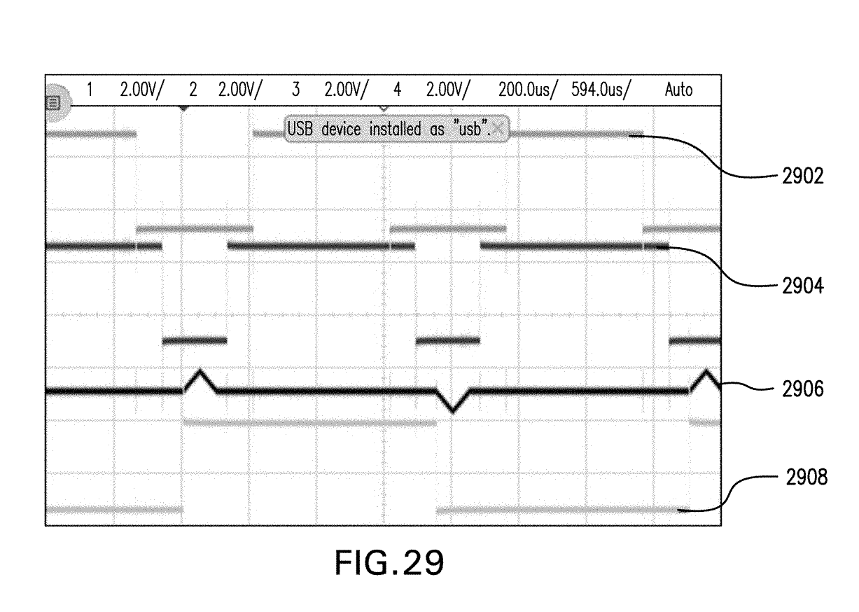

FIG. 20B shows example timing of gradient detection, and signal-measurement components. An example system 2024 is shown, and, in plot 2026 a graphical representation of the gradients is shown. As shown, the gradient changes are detected (plot 2028), and are used to provide switching triggers (plot 2030) that control the switching circuit (2004, FIG. 20A) and the variable gain amplifier (2006, FIG. 20A). A further detailed switching sequence in shown in FIG. 29.

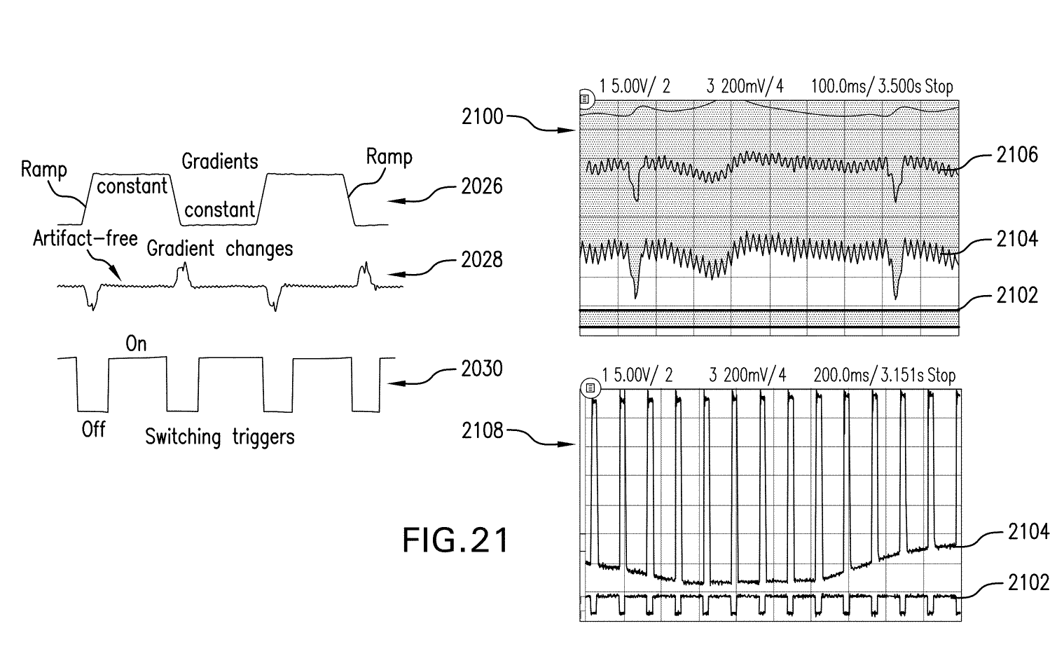

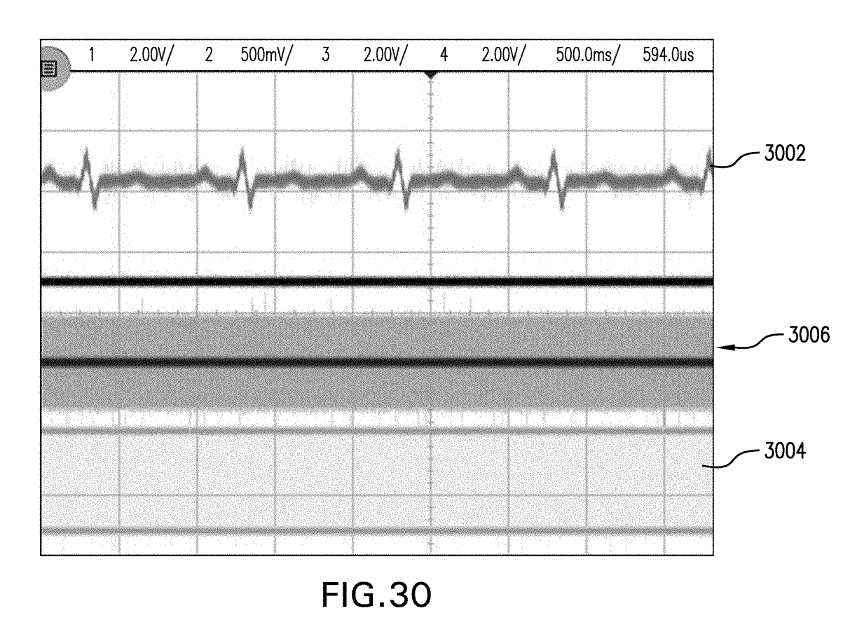

FIG. 21 shows results 2100 provided by an example variable gain circuit, and related data. Shown are the triggering signal 2102, the raw ECG signal 2104, and the resampled ECG signal 2106 avoiding the gain switching interval. To diminish the effect of artifacts and reduce switching noise, a variable gain analog circuit can be used, e.g., as in Illustrative Features (iii), #1, or #7. In response to the gradient information (#6), this circuitry (e.g., control unit 514, 614, 2012) controls the SPDT switches 2004 (FIG. 20A) and also modulates the gain of the analog circuit 2006 (FIG. 20A) so that, during the presence of electromagnetic field variation, the amplification is reduced, and during electrophysiological signal recording it is increased. For example, the gain during recording can be about 500× the gain during field variation. This can reduce the magnitude of the gradient-induced artifacts, and of switching artifacts from the analog switches 504, 618, 2004. This can also reduce saturation of the amplifier, as shown in FIG. 30. Also shown in FIG. 21 is a plot 2108 showing a magnified representation of the original signal, the raw ECG signal 2104, and the triggering signal 2102.

FIGS. 22A and 22B show example EEG configurations of differential signaling for unipolar recording. In the illustrated example, active and differential transmission of the reference signal cancels out the effects of the electromagnetic interference. Techniques shown in FIGS. 22A and 22B can additionally or alternatively be used for measuring EP signals other than EEG signals. In some examples of EP data capture, unipolar or bipolar recordings can be captured. In unipolar configurations, the reference electrode 2208 can be near the neck, or otherwise away from the skull, e.g., away from the recording or active electrodes. In some examples, differences between the lengths of the reference electrode 2208 and active electrode 2204 can cause differences in artifacts induced by the changing magnetic fields. Accordingly, as shown in FIGS. 22A and 22B, the reference signal can be carried via a differential pair 2206 to an amplifier (depicted as an op-amp) located at the test electrode 2204, or vice versa. In some examples, the reference signal is connected to the amplifier, as is the active signal. In some examples, the reference signal is carried via a differential pair 2206 to substantially where the active signal is captured 2204. Differential pair 2206 can reduce artifacts and EMI, as discussed herein with reference to Illustrative Features (iii) and (iv).

In some examples using differential signaling, the EEG signals can be sensed through bipolar or unipolar configurations. For bipolar recordings, a twisted pair of wires can be used. The length of these wires can be reduced as the active device is placed near to the recording site and as a result, induced artifacts are reduced considerably. In case of unipolar recording, the reference potential (between the reference and ground electrodes) can be carried differentially, using respective twisted pair(s) of wires, to local electrode(s). The use of active differential signaling serves to substantially cancel out the electromagnetic interference along the wired connections between reference electrode and the active electrode(s).

FIG. 22B shows a reference unit (dashed box). The reference unit includes a reference electrode 2208 (“REF”) configured to contact the body of a subject and to provide a signal. For example, the electrode can be an EP measurement electrode such as an EEG electrode. The signal can be digital or analog.

The reference unit includes a signal transmission unit 2214 configured to transmit the signal via a differential pair 2206. In the illustrated example, the reference unit includes an amplifier (“Amp”) to amplify the signal from the reference electrode, and a differential driver (depicted as a buffer and an inverter sharing a common input, although the driver can be digital or analog, and can be voltage-mode or current-mode) to drive the amplified signal on the differential pair 2206. The differential pair 2206 can use various types of cable, e.g., flat ribbon, twin axial, or twisted-pair.

The reference unit also includes a differential to single ended converter (referred to for brevity as a “balun” or “DS converter” and depicted as an op-amp), configured to provide a reconstructed reference signal 2202. In the illustrated example, the DS converter (“balun”) includes an amplifier, e.g., a differential amplifier, fed with the differential pair 2206 as its + and − inputs. The balun/DS converter can additionally or alternatively include a transformer, choke, or other component for converting balanced signals to unbalanced signals (hence “bal”-“un”) or differential to single-ended signals (hence “D” and “S” in “DS converter”).

FIG. 22B and FIGS. 5, 9, 11, 15, 16, 20A, 20B, and 43 show components of measurement circuitry configured to detect, from within a magnetic resonance imaging (MRI) bore, magnetic field changes due to the operation of MRI coil(s). The measurement circuitry is further configured to isolate detection circuitry from transients during the magnetic field changes, e.g., using switching circuits and variable gain amplification described herein with reference to FIG. 20A. The measurement circuitry is further configured to measure an electrophysiological signal, e.g., at the electrode 2216 in FIG. 22B. The EP signal can be measured based on the reconstructed reference signal 2202 and using the detection circuitry at a time other than during the magnetic field changes. For example, the measurement circuitry can measure the EP signal based on a difference between a signal at the reference electrode 2208 (“REF”) and a signal at the active electrode 2216. In some examples, when the switching triggers of FIG. 20B are on, the variable gain amplifier can amplify a difference between the reconstructed reference signal 2202 and the signal measured at the right-hand electrode in FIG. 22B.

In some examples, the measurement circuitry is configured to detect an activity period of the MRI coil(s), e.g., as discussed herein with reference to FIG. 5, 8, 15, 16, 18, 20A, 20B, 21, 24, 25, 29, 30, 31, 37, 38, or 43. In some examples, the measurement circuitry is configured to transmit data corresponding to the electrophysiological signal during the activity period. Examples are discussed herein, e.g., with reference to FIG. 5, 6, 9-12, 17, 20A, 23, 26-28, 37, or 38.

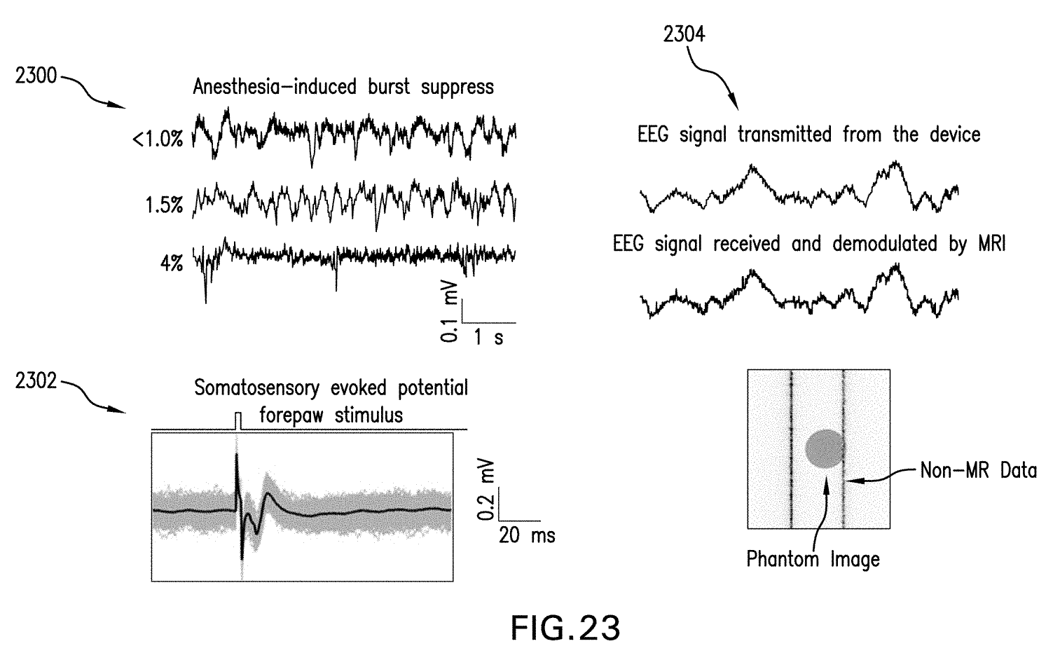

FIG. 23 shows example phantom and animal data that was collected within an MRI bore. Shown on the upper left side (plot 2300) is a RAT LFP observed with active sensing and wireless transmission. As seen, spontaneous LFP changed progressively with deeper anesthesia (isoflurane) towards burst suppression. Forepaw-stimulus-evoked LFP in the somatosensory cortex is shown on the bottom left (plot 2302). On the right side is shown an example of the fidelity of transmission and reconstruction of the data (plot 2304), where a high fidelity electrophysiological signal (EEG) extracted from raw-MM data through demodulation (bottom) is compared with the transmitted EEG signal (top) and shown to match. Non-MR data appear as strips in the extended FOV (bottom-right image).

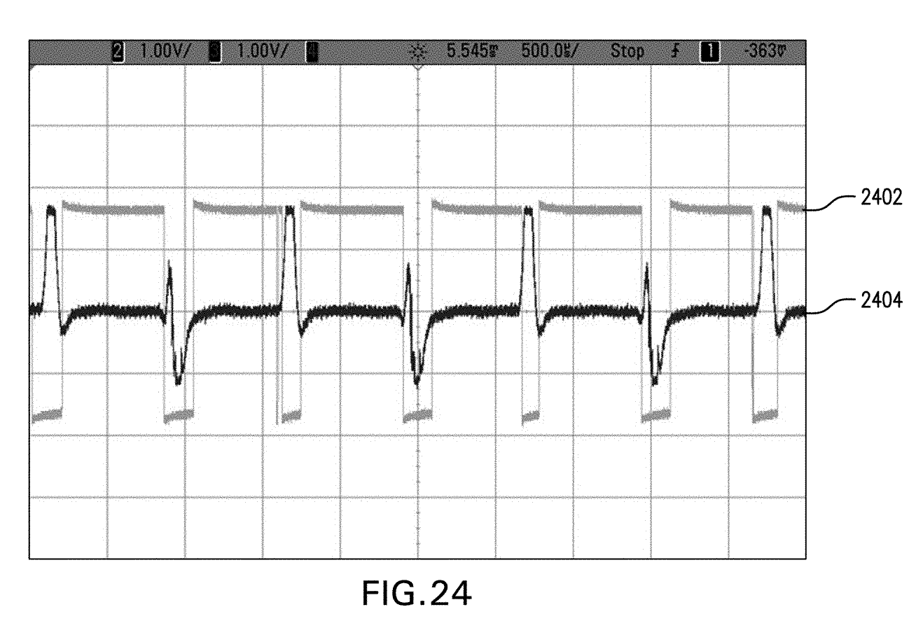

FIG. 24 shows wireless gradient detection of example triggering signals that were determined within an MRI bore during an MRI scan. The triggering signal 2402 for the analog and digital circuitry is shown along with the gradient signal 2404 picked up through the coil 522. As shown, the high levels of triggering signal 2402 generally correspond with regions between changes in the gradient signal 2404.

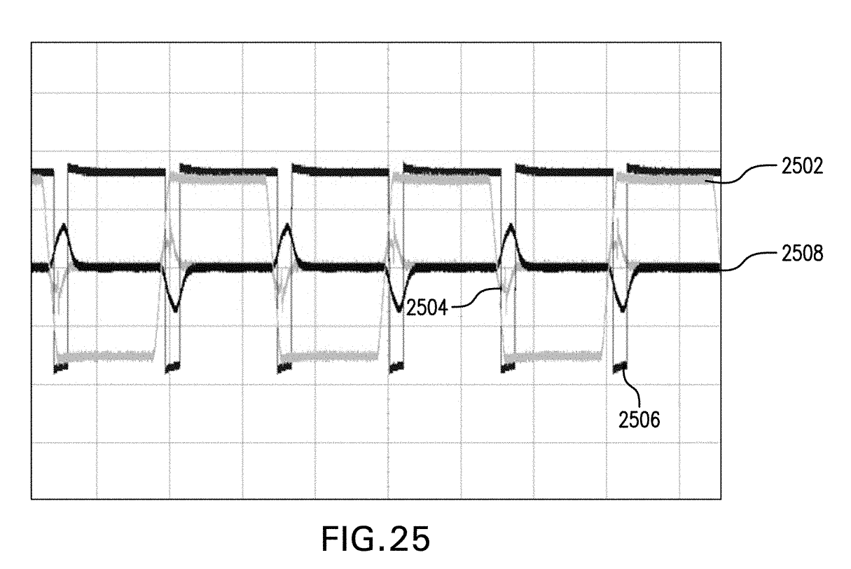

FIGS. 25-33 show further examples of data that were measured in various experiments or that were simulated.

FIG. 25 shows the gradient trigger 2502 from the MRI and the signal 2504 from the pickup coil in comparison with the filtered signal 2508 and the generated sampling/switching triggers 2506.

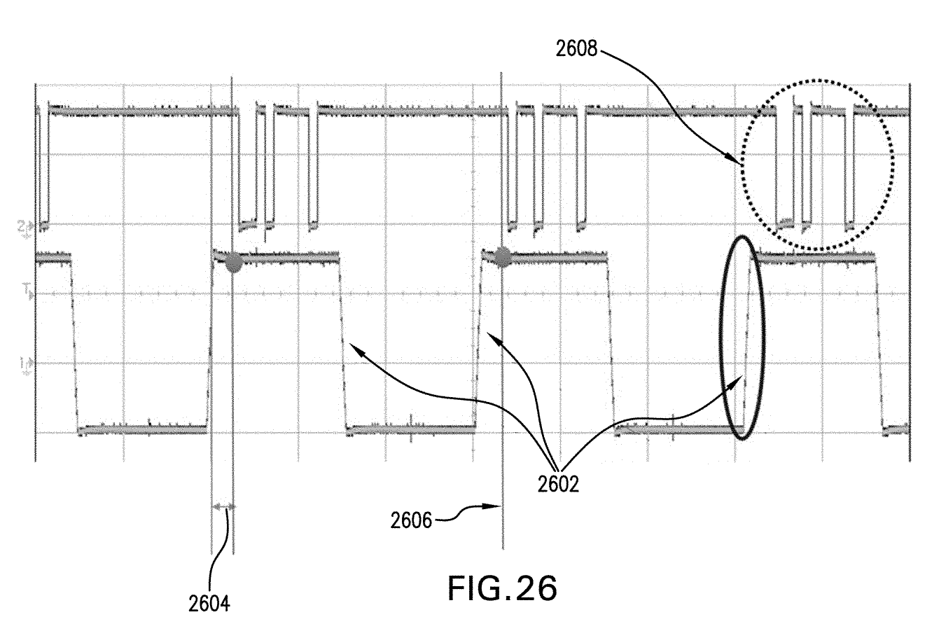

FIG. 26 shows an example in which sampling and transmission are synchronized with the gradient field. After a rapid change 2602 in the magnetic field, the control unit 514, 614, 2012 delays (period 2604) to wait for the gradient artifact to die down. After the magnetic field has been determined to be, or has become, substantially steady (time 2606), the process samples the data and then sends the digitized data 2608 to the transmitter.

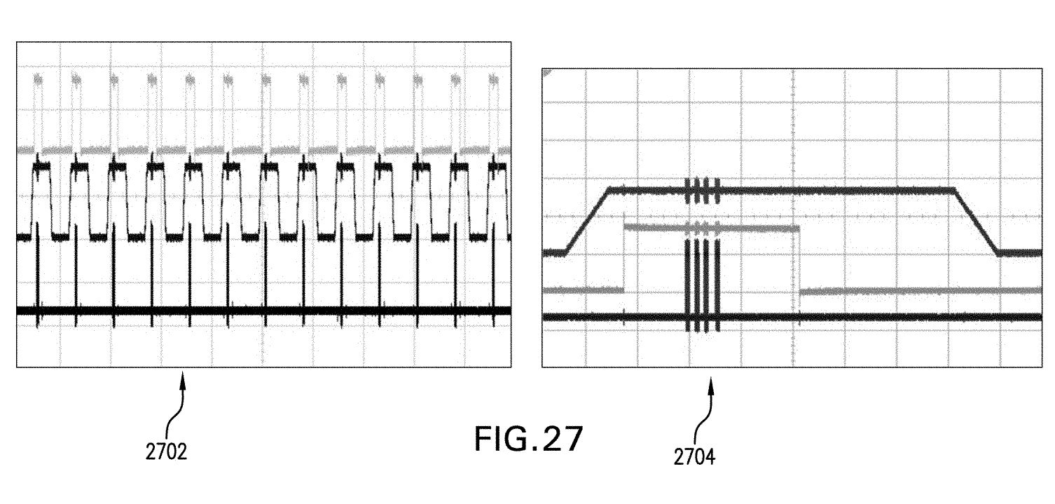

FIG. 27 shows an example of synchronized sampling. In graph 2702, the ADC turn-on signal from the MCU is shown and in graph 2704 the ADC sampling clock is shown.

FIG. 28 shows an example of modulation of MRI data and wireless data reconstruction. Digital data reconstruction is shown in graph 2802 and filtered MR-raw data for FSK demodulation is shown in graph 2804.

FIG. 29 shows an example of the switching trigger and simulated noise. Shown in the graph is the variable gain trigger 2902, the analog switch trigger 2904, the simulated gradient artifact 2906 (at 1.5 Vpp), and the simulated gradient artifact trigger 2908.

FIG. 30 shows an example of recovering simulated ECG from a signal severely corrupted by gradient artifact. Shown in the graph is the recovered ECG signal 3002, the signal corrupted by the gradient artifact 3006, and the simulated gradient artifact trigger 3004. The actual simulated ECG signal has an amplitude of 4 mVpp, and is hidden by the gradient artifact of 1.5 Vpp.

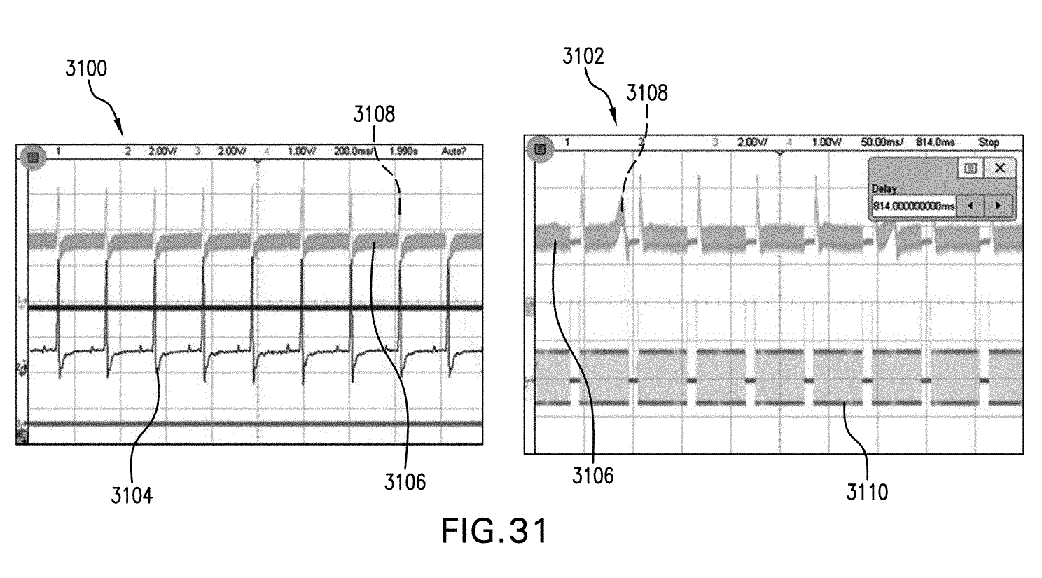

FIG. 31 shows gradient artifact free recording of a RAT ECG during concurrent fMRI acquisition. A comparison is displayed between a RAT ECG within an MRI that is off iso-center without fMRI (plot 3100) and a RAT ECG during an fMRI at the iso-center (plot 3102). The signal 3104 after the digital low pass filter can be seen in graph 3100. The P-wave 3106 in the RAT ECG can be seen in in both graphs along with the QRS complex 3108 in the RAT ECG. The gradient trigger signal 3110 from the MR-scanner is also shown in graph 3102.

FIG. 32 shows a detailed graph of biphasic stimulation pulses of the current stimulator while in a burst mode. The upper-right plot shows a delay of 2.42796 s. The lower plot shows a delay of 2.43216 s.

FIG. 33 shows variable pattern generation of current stimulation. The graphs represent encoding an M-sequence in stimulation to obtain an averaged response with minimized session duration. M-sequences can be used to estimate the impulse response of a linear time-invariant (LTI) system using a relatively small amount of data.

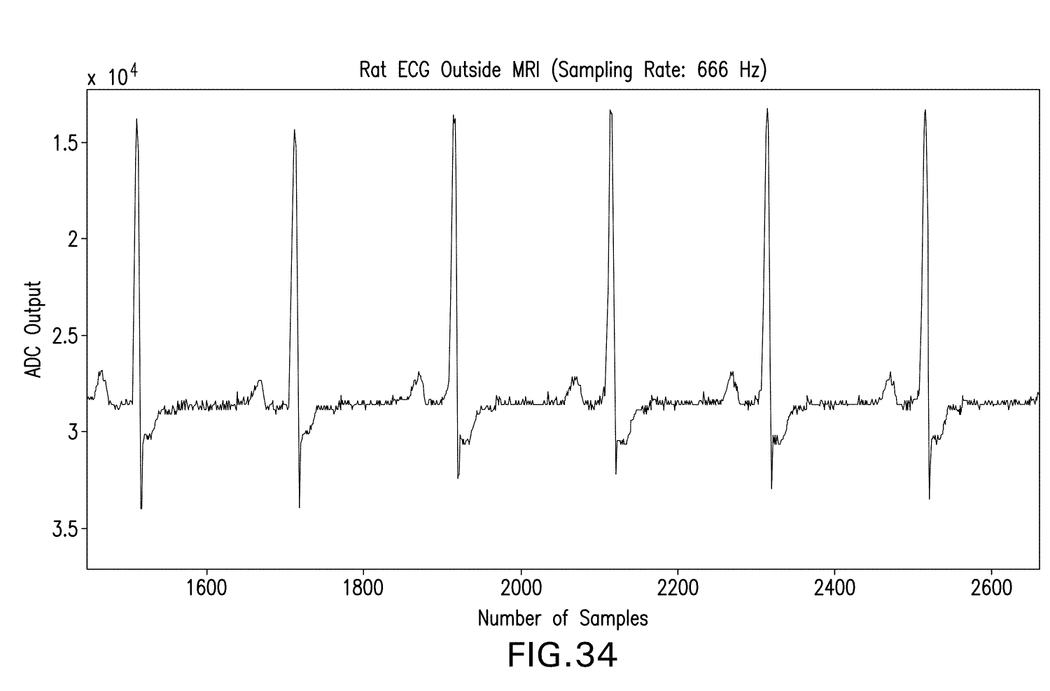

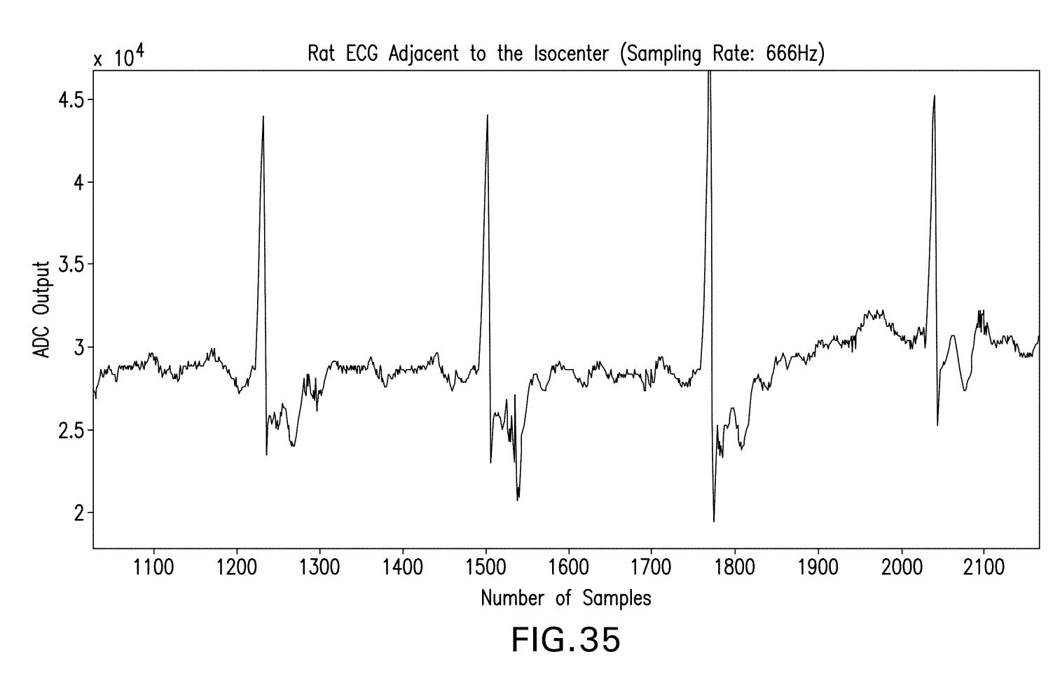

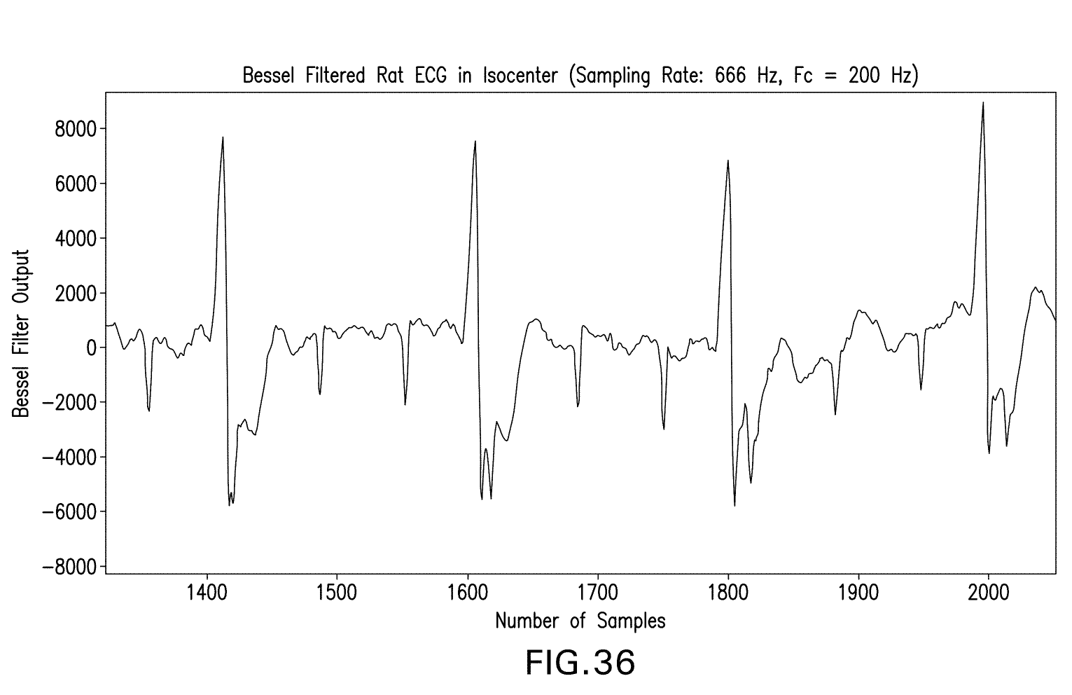

FIGS. 34, 35, and 36 show example electrocardiogram data that was collected from a rat. The data in FIG. 34 were collected outside an MRI. The data in FIGS. 35 and 36 were collected within the MRI bore, during an fMRI scan, using techniques described herein.

In some examples, a “control unit” as described herein includes processor(s) 1786. A control unit can also include, if required, data storage system 1740 or portions thereof. For example, a control unit can include (1) a CPU or DSP and (2) a computer storage medium or other tangible, non-transitory computer-readable medium storing instructions executable by that CPU or DSP to cause that CPU or DSP to perform functions described herein. Additionally or alternatively, a control unit can include an ASIC, FPGA, or other logic or circuit device(s) wired (e.g., physically, or via blown fuses or logic-cell configuration data) to perform functions described herein. For example, a control unit can comprise the amplifier, filter, comparator, and logic-signal generator of circuitry 2024, FIG. 20B. In some examples of control units including ASICs or other devices physically configured to perform operations described herein, a control unit does not include computer-readable media storing executable instructions. In some examples, a control unit includes (1) a program-executing device (e.g., a CPU or DSP) and a computer-readable medium, and (2) a hard-wired device (e.g., an FPGA or circuitry block, e.g., circuitry 2024 excluding the pick-up coil). The program-executing device and the hard-wired device can be communicatively connected and can interoperate to perform functions described herein.

FIG. 37 shows an example pulse sequence. In some examples, the MR scanner 532 captures MR data (RF echo data from subject 528), and simultaneously captures EP data transmitted by communication module 526 in, e.g., a different frequency band, as discussed herein with reference to FIG. 19.

In some examples, MR control system 534 is configured to decode non-MR data, e.g., upon receipt, and control the operation of the MR scanner 532 accordingly. For example, a system 500, 600 can transmit non-MR data including a control signal, e.g., during at least one readout phase, e.g., of the three illustrated readout phases. MR control system 534 can detect the control signal in the non-MR data, and set timing, slice, or other parameters of operation of MR scanner 532 according to, or in response to, the control signal.

In some examples, system 500, 600 detects a physiological event based on the measured EP data. For example, system 500, 600 can determine a trigger point in a QRS cycle based on ECG data. For example, the trigger point can be the peak of the R wave. In response, system 500, 600 can transmit the control signal indicating that an MR scan should be conducted. The MR control system 534 can commence an MR scan using MR scanner 532 in response to receipt of the control signal. In some examples, system 500, 600 can detect the event by matching the detected EP signals to a pattern, by performing a running correlation test between the EP signals and a pattern, by using locality-sensitive hashing of a window of EP signals and an expected pattern, by detecting transients (e.g., using differentiation or other peak-detection techniques), or by detecting signal levels or swings within predetermined ranges (e.g., a magnitude of a value or change exceeding a threshold). ECG is an example; EEG or other types of EP signals can additionally or alternatively be used in determining trigger points.

In some examples, a control unit can determine readout periods as described herein, e.g., using timers or detection of gradient signals as described below. In some example, the control unit can determine, for a particular quiescent period, whether that quiescent period is a readout period. For example, the control unit can determine the intersection between times of quiescent periods and times of readout periods, e.g., via linear search or interval-tree search. The control unit can then transmit data, provide stimulation, or perform other activities that might introduce noise in MR measurements, during (e.g., only during) quiescent periods that are not MR readout periods.

FIG. 38 shows example pulse sequences. In some examples, the illustrated pulse sequences are used with MR scanners 532 having readout coils sensitive only in a single band, although this is not required. Pulse sequence 1 is used to conduct the MR imaging. During the illustrated “sampling zones” in pulse sequence 1, the EP recording system 500, 600 captures the EP signal of interest and stores the digitized data on an onboard memory unit (i.e. flash memory or other computer-readable media). System 500, 600 can use the above-described RF and gradient pulse avoidance system (e.g., gradient detection 612 and control unit 614) to capture the EP signal substantially without gradient-induced artifacts. Also during pulse sequence 1, the MR scanner's RF coil(s) behave as usual to first excite the polar molecules inside the subject then receive the echoed RF energy released from those molecules to generate an MR image. In the illustrated example, no non-MR (EP) signals are sent during pulse sequence 1.

After pulse sequence 1, pulse sequence 2 can be carried out. During pulse sequence 2, the RF coil is operated to only receive (e.g., no RF excitation or RF pulses are generated by the RF coil). In some examples, a steady readout gradient is maintained during readout (as depicted by the white hexagon); in other examples, a readout gradient is not maintained during readout. The system 500, 600 transmits the stored digitized data, e.g., in any frequency band(s) supported by the MRI (e.g., using the full readout bandwidth of MR scanner 532). Since MR echoes are substantially absent due to the time lapse since the conclusion of RF excitation, the EP signals can be transmitted with substantially no interference from or to the MR signals.

In some examples, adaptive pulse sequences as described herein with reference to Illustrative Feature #7 can be used with at least one of pulse sequence 1 or pulse sequence 2. Measurement of gradient-edge timing as discussed herein can be used to determine the present pulse sequence, and the present point within that pulse sequence. In some examples of pulse sequence 2, gradient pulses can be generated (e.g., represented by the hollow hexagon on G_{readout}) by the MR scanner to request that the MCU begin transmission of EP data. The triggering circuit in the MCU (or other control unit) can detect the changing gradient during pulse sequence 2 and trigger the transmission. In other examples of pulse sequences 1 and 2, the MCU can transmit on a schedule rather than in response to a gradient pulse, e.g., based on pre-programmed information of the timing between pulses in pulse sequence 1 and the readout window in pulse sequence 2. Accordingly, pulse sequence 2 can involve a delay time during which no pulses occur, in some examples. Alternatively, pulse sequence 2 can involve a time period during which at least one pulse does occur.

Pulse sequences 1 and 2 can be alternated repeatedly to conduct concurrent MR and EP detection and readout. The EP (non-MR) data can be timestamped at the point of acquisition by the MR scanner 532. The recorded, timestamped EP data then can be correlated with the MR image data in a post processing stage, e.g., via a fuzzy table lookup or nearest-neighbor search based on the timestamps of MR images and EP data.

Continuing the example of control signals described herein with reference to FIG. 37, in some examples, the MR scanner 532 and MR control system 534 can detect control signals using the readout coils of MR scanner 532 even when no MR scan is active. This can permit detecting control signals, e.g., during periods in which the MR scanner 532 is idle or in standby. This can reduce EMI in the detection of EP signals and in the transmission of control signals. For example, control signals can be detected during the readout portion of pulse sequence 2, or at a time when no gradient is being applied.

FIG. 39 is a graphical representation of image data of a phantom image including non-MR data (visible as specks at the side).

FIG. 40 is a graphical representation of image data of (top) a phantom image including encoded non-MR data, and (bottom) a rat-brain image including encoded non-MR data.

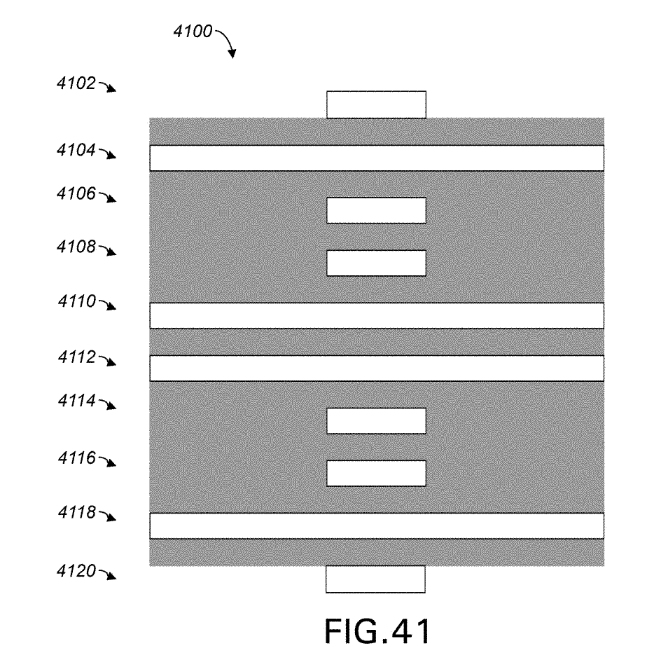

FIG. 41 shows an example circuit-board stackup 4100 (profile section) that can be used in preparing measurement systems 1725 or other electrical components designed for use in an MR bore. Systems using the illustrated stackup can experience reduced EMI compared to some prior schemes. In some examples, outer layers 4102 and 4120 can carry relatively lower-frequency signals, layers 4104 and 4118 can carry ground (GND) (e.g., ground planes), layers 4110 and 4112 can carry power or ground (e.g., VCC or GND planes), and inner layers 4106, 4108, 4114, and 4116 can carry relatively higher-frequency signals.

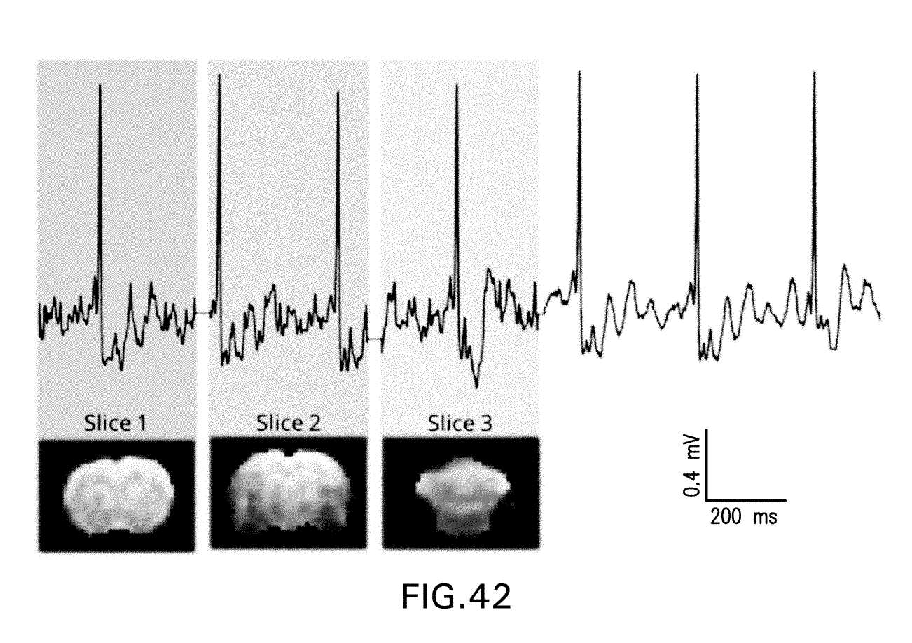

FIG. 42 shows a rat ECG observed using active sensing, together with several corresponding MRI slices.

FIG. 43 shows an example wireless-detection and powering module, which can include at least one of a power-harvesting subsystem 4302 and a gradient-detection subsystem 4312. At least one of power-harvesting subsystem 4302, which can represent power-harvesting module 516, or gradient-detection subsystem 4312, which can represent block 518 or 612, can include a pick-up coil 4304, e.g., coil 522, and a rectifier 4306.

The power-harvesting subsystem 4302 can include a DC-DC converter 4308, e.g., a boost or buck converter or a charge pump, to change the overall voltage levels from the rectifier 4306. A regulator 4310 then provides a stable VCC level (with respect to a system ground). In some examples, DC-DC converter 4308 and regulator 4310 are combined in a single block, e.g., a switched-mode power supply.

The gradient-detection subsystem 4312 can include an amplifier 4314 (gain over- or under-unity) feeding a filter 4316. A comparator 4318 can compare the output of the filter 4316 to a predetermined reference level or an automatically-adjusted reference level, e.g., as discussed herein with reference to FIG. 4 or 19 or components 612, 2016, or 2024. A logic signal generator 4320, e.g., a Schmitt-triggered buffer, can provide a logic signal indicating when gradients are present. In some examples, the reference level can be set by determining a peak of the output of the filter 4316 (e.g., over a predetermined time window); filtering the detected peaks through an RC filter to provide a filtered signal having a smoother response, based on a predetermined time constant; and providing the filtered signal to an automatic gain control (AGC) unit to provide the reference level.

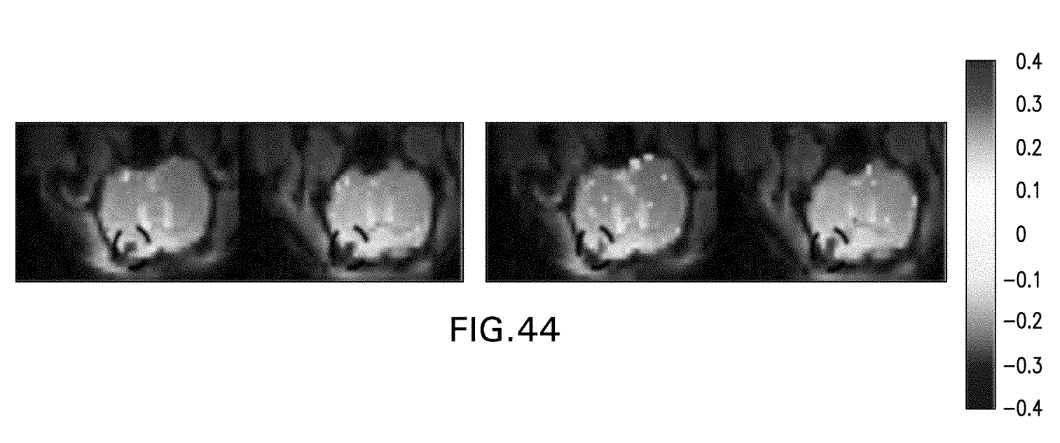

FIG. 44 shows graphical representations of MR images. On the left are shown MR images produced using a conventional MR scanner. The right side shows concurrent imaging and recording (“MR-link operation”) of somatosensory evoked responses. These data demonstrate that the tested EP measurement system was MR-compatible and able to provide data during an MRI process.

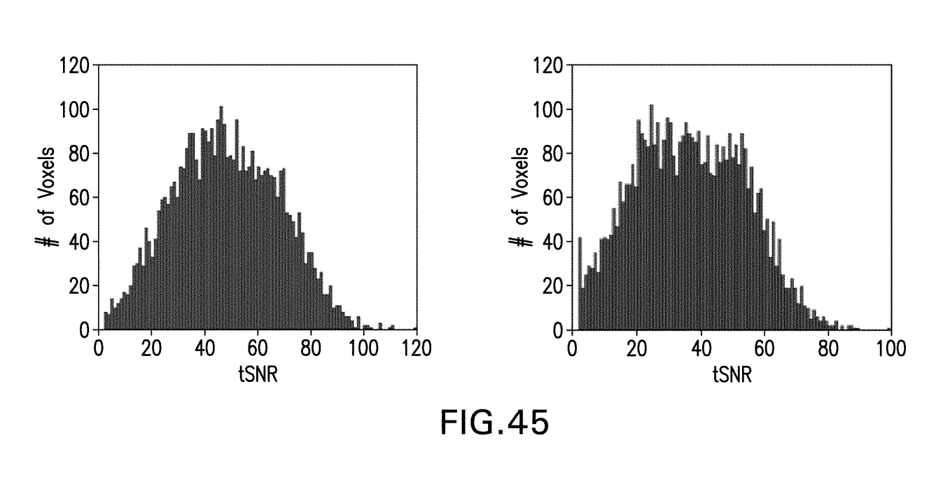

FIG. 45 shows graphics of temporal SNR. Normal operation is shown at left; MR-link operation is shown at right. These data also evidence MR compatibility of the EP measurement system.

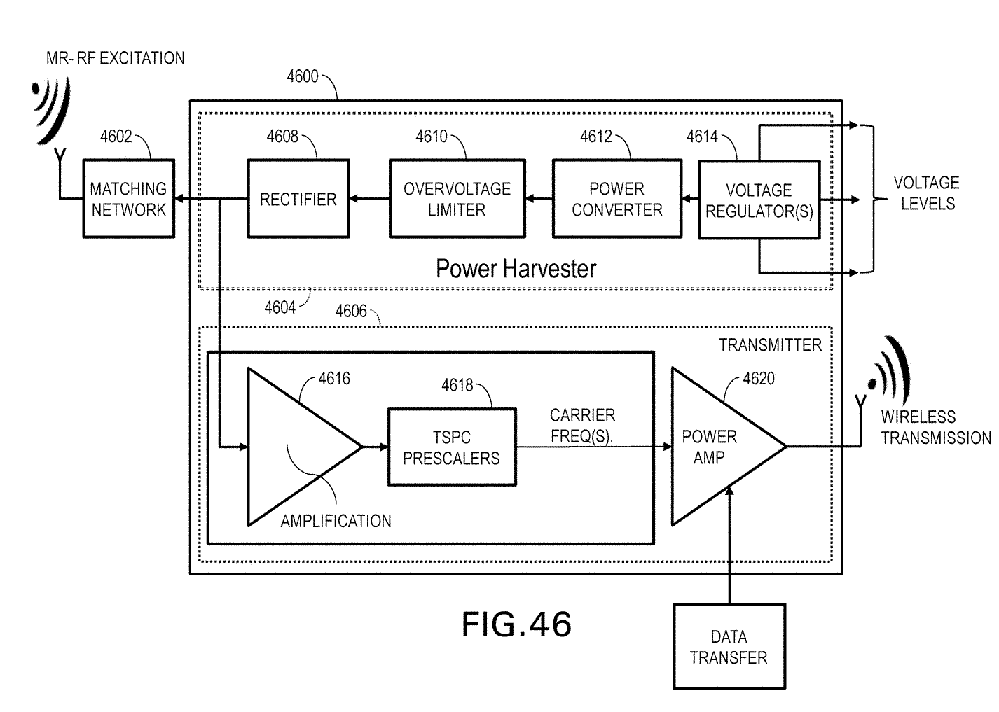

FIG. 46 shows an example subsystem 4600 for reference-frequency generation or power harvesting. Subsystem 4600 can include matching network 4602 feeding at least one of power-harvesting block 4604 (which can represent module 516), and transmitter block 4606 (which can represent transmitter 520). In some examples, transmitter block 4606 can be implemented using dedicated integrated circuits. Transmitter block 4606 can generate carrier frequencies based on RF excitation provided by the MR scanner 532. RF energy during MR-excitation is passed through a matching network 4602, e.g., a matched filter, to isolate the target frequency.

Power-harvesting block 4604 can include rectifier 4608 (e.g., rectifier 4306), overvoltage limiter 4610, and power converter 4612 (e.g., DC-DC converter 4308) electrically connected in series. The output of power converter 4612 can feed one or more regulators 4614 (e.g., regulator 4310) to provide DC output voltages required by other components of the system (e.g., 3.3V, 5V, or other logic levels).

Transmitter block 4606 can include one or more amplification stages 4616. The amplification stages can feed one or more true single phase clocked (TPSC) frequency prescalers 4618 (or prescalers implemented using other technologies) that generate desired carrier frequencies for the transmitter 520. In some examples, pre-amplifiers and power amplifiers (e.g., power amplifier 4620) can be used for various multi-frequency transmission schemes e.g. OFDM, CDMA etc. This can reduce the power required for a data transmission scheme, permitting operation in wireless devices.

### Example Clauses

Various examples include one or more of, including any combination of any number of, the following example features. Throughout these clauses, parenthetical remarks are for example and explanation, and are not limiting. Parenthetical remarks given in this Example Clauses section with respect to specific language apply to corresponding language throughout this section, unless otherwise indicated.

A: A system, comprising: at least one conductive coil; a reference unit comprising: a reference electrode configured to contact the body of a subject and to provide a signal; a signal transmission unit configured to transmit the signal via a differential pair; and a balun/DS converter configured to provide a reconstructed reference signal; and measurement circuitry configured to: detect, from within a magnetic resonance imaging (MM) bore, magnetic field changes due to the operation of MRI coil(s); isolate detection circuitry from transients during the magnetic field changes; measure an electrophysiological (EP) signal based on the reconstructed reference signal and using the detection circuitry at a time other than during the magnetic field changes; detect an activity period of the MRI coil(s); and transmit data corresponding to the EP signal during the activity period. (In some examples, paragraph A can additionally or alternatively include not detecting the activity period of the MRI coil(s), and can include transmit data corresponding to the EP signal to the MRI coil(s).)

B: The system according to paragraph A, further comprising: a programmable stimulation module configured to provide at least one of electrical current or electromagnetic radiation to tissues of a subject.

C: The system according to paragraph B, wherein the programmable stimulation module is configured to provide the at least one of electrical current or electromagnetic radiation at a time other than during the magnetic field changes. (In some examples, paragraph C can additionally or alternatively include providing the at least one of electrical current or electromagnetic radiation, with an option to deliver the stimulation only at times other than during the magnetic field changes.)

D: The system according to any of paragraphs A-C, wherein the programmable stimulation module is configured to provide the at least one of electrical current or electromagnetic radiation corresponding with a user-defined stimulation pattern.

E: The system according any of paragraphs A-D, further comprising: a wireless power harvesting module configured to: receive electromagnetic energy within the MR bore; transform the received electromagnetic energy to electrical energy; and provide the electrical energy to at least one other component of the device to power the at least one other component.

F: The system according to paragraph E, wherein the at least one other component comprises at least one of a stimulation module or a recording module.

G: The system according to any of paragraphs A-F, wherein the measurement circuitry comprises a variable gain amplifier and the measurement circuitry is configured to reduce the gain during the operation of the MRI coil(s).

H: The system according to any of paragraphs A-G, wherein the measurement circuitry: comprises at least one active electrode configured to contact the body of the subject and to provide an active signal; and is configured to provide the EP signal based on the reconstructed reference signal and the active signal.

I: The system according to any of paragraphs A-H, wherein the system is magnetic-resonance (MR)-compatible.

J: The system according any of paragraphs A-I, further comprising: at least one processor; and a memory storing instructions that, when executed by the at least one processor, cause the at least one processor to perform operations comprising at least one of: demodulating, displaying, or analyzing measured EP signal(s).

K: Methods as described herein for performing operations comprising at least one of: measuring EP signals or demodulating, displaying, or analyzing measured EP signal(s).

L: Computer-readable media as described herein having thereon processor-executable instructions for performing operations comprising at least one of: measuring EP signals or demodulating, displaying, or analyzing measured EP signal(s).

M: A computer-readable medium, e.g., a computer storage medium, having thereon computer-executable instructions, the computer-executable instructions upon execution configuring a computer to perform operations as any of paragraphs A-J recites.

N: A device comprising: a processor; and a computer-readable medium, e.g., a computer storage medium, having thereon computer-executable instructions, the computer-executable instructions upon execution by the processor configuring the device to perform operations as any of paragraphs A-J recites.

O: A system comprising: means for processing; and means for storing having thereon computer-executable instructions, the computer-executable instructions including means to configure the system to carry out a method as any of paragraphs A-J recites.

N: A system, comprising: at least one conductive coil; a reference unit comprising: a reference electrode configured to contact the body of a subject and to provide a signal; a signal transmission unit configured to transmit the signal via a differential pair; and a DS converter configured to provide a reconstructed reference signal; and measurement circuitry configured to: detect, from within a magnetic resonance imaging (MM) bore, magnetic field changes due to the operation of MRI coil(s); isolate detection circuitry from transients during the magnetic field changes; measure an electrophysiological (EP) signal based on the reconstructed reference signal and using the detection circuitry at a time other than during the magnetic field changes; detect an activity period of the MRI coil(s); and transmit data corresponding to the EP signal during the activity period. (In some examples, paragraph N can additionally or alternatively include not detecting the activity period of the MRI coil(s), and can include transmit data corresponding to the EP signal to the MRI coil(s).)

O: The system according to paragraph N, further comprising: a programmable stimulation module configured to provide at least one of electrical current or electromagnetic radiation to tissues of a subject.

P: The system according to paragraph O, wherein the programmable stimulation module is configured to provide the at least one of electrical current or electromagnetic radiation at a time other than during the magnetic field changes. (In some examples, paragraph P can additionally or alternatively include providing the at least one of electrical current or electromagnetic radiation, with an option to only stimulate at times other than during the magnetic field changes.)

Q: The system according to any of paragraphs N-P, wherein the programmable stimulation module is configured to provide the at least one of electrical current or electromagnetic radiation corresponding with a user-defined stimulation pattern.

R: The system according any of paragraphs N-Q, further comprising: a wireless power harvesting module configured to: receive electromagnetic energy within the MR bore; transform the received electromagnetic energy to electrical energy; and provide the electrical energy to at least one other component of the device to power the at least one other component.

S: The system according to paragraph R, wherein the at least one other component comprises at least one of a stimulation module or a recording module.

T: The system according to any of paragraphs N-S, wherein the measurement circuitry comprises a variable gain amplifier and the measurement circuitry is configured to reduce the gain during the operation of the MRI coil(s).

U: The system according to any of paragraphs N-T, wherein the measurement circuitry: comprises at least one active electrode configured to contact the body of the subject and to provide an active signal; and is configured to provide the EP signal based on the reconstructed reference signal and the active signal.

V: The system according to any of paragraphs N-U, wherein the system is magnetic-resonance (MR)-compatible.

W: The system according any of paragraphs N-V, further comprising: at least one processor; and a memory storing instructions that, when executed by the at least one processor, cause the at least one processor to perform operations comprising at least one of: demodulating, displaying, or analyzing measured EP signal(s).

X: Methods as described herein for performing operations comprising at least one of: measuring EP signals or demodulating, displaying, or analyzing measured EP signal(s).

Y: Computer-readable media as described herein having thereon processor-executable instructions for performing operations comprising at least one of: measuring EP signals or demodulating, displaying, or analyzing measured EP signal(s).

Z: A computer-readable medium, e.g., a computer storage medium, having thereon computer-executable instructions, the computer-executable instructions upon execution configuring a computer to perform operations as any of paragraphs N-X recites.

AA: A device comprising: a processor; and a computer-readable medium, e.g., a computer storage medium, having thereon computer-executable instructions, the computer-executable instructions upon execution by the processor configuring the device to perform operations as any of paragraphs N-X recites.

AB: A system comprising: means for processing; and means for storing having thereon computer-executable instructions, the computer-executable instructions including means to configure the system to carry out a method as any of paragraphs N-X recites.

AC: A system, comprising: one or more antennas; a reference unit comprising: a reference electrode configured to contact the body of a subject and to provide a signal; a signal transmission unit configured to transmit the signal as two differential signals via a differential pair; and a converter configured to receive the two differential signals via the differential pair and to provide a reconstructed reference signal based at least in part on the two differential signals; measurement circuitry configured to measure an electrophysiological (EP) signal of the subject based at least in part on the reconstructed reference signal; detection circuitry configured to: detect, using at least one of the one or more antennas, magnetic-field changes due to the operation of magnetic resonance (MR) coil(s); and isolate the detection circuitry from electrical transients during the magnetic-field changes; a control unit configured to: operate the detection circuitry to measure the EP signal at a time other than during the magnetic-field changes; and a communication module configured to: transmit data corresponding to the EP signal via at least one of the one or more antennas.

AD: The system according to paragraph AC, further comprising: a programmable stimulation module configured to provide at least one of electrical current or electromagnetic radiation to tissues of a subject.

AE: The system according to paragraph AD, wherein the control unit is configured to operate the programmable stimulation module to provide the at least one of electrical current or electromagnetic radiation at a time other than during the magnetic field changes.

AF: The system according to any of paragraphs AC-AE, wherein the programmable stimulation module is configured to provide the at least one of electrical current or electromagnetic radiation corresponding with a predetermined stimulation pattern.

AG: The system according any of paragraphs AC-AF, further comprising: a wireless power harvesting module configured to: receive electromagnetic energy via at least one of the one or more antennas; transform the received electromagnetic energy to electrical energy; and provide the electrical energy to at least one other component of the device to power the at least one other component of the device, wherein the at least one other component comprises at least one of a stimulation module, a recording module, the reference unit, the detection circuitry, the measurement circuitry, the control unit, or the communication module.

AH: The system according to any of paragraphs AC-AG, wherein the detection circuitry comprises a variable gain amplifier and the detection circuitry is configured to reduce the gain during the operation of the MRI coil(s).

AI: The system according to any of paragraphs AC-AH, wherein the measurement circuitry: comprises at least one active electrode configured to contact the body of the subject and to provide an active signal; and is configured to provide the EP signal based on the reconstructed reference signal and the active signal.

AJ: A device, comprising: one or more antennas; an operation unit comprising at least one of an electrophysiological (EP) detection unit or a stimulation unit; and a control unit configured to: detect changes to a magnetic field around the device; isolate the operation unit from transients during the magnetic-field changes; and activate the operation unit at a time other than during the magnetic-field changes.

AK: The device according to paragraph AJ, wherein: the operation unit comprises the EP detection unit configured to, when activated, measure an electrophysiological (EP) signal of a subject; and the control unit is further configured to: detect a readout period based at least in part on the changes to the magnetic field; and transmit data corresponding to the electrophysiological signal via at least one of the one or more antennas during the readout period.

AL: The device according to paragraph AJ or AK, wherein: the operation unit comprises the stimulation unit configured to, when activated, provide at least one of electrical current or electromagnetic radiation to tissues of a subject.

AM: The device according to any of paragraphs AJ-AL, further comprising: a wireless power harvesting module configured to: receive electromagnetic energy within the MR bore; transform the received electromagnetic energy to electrical energy; and provide the electrical energy to at least one other component of the device to power the at least one other component, wherein the at least one other component comprises at least one of a stimulation module, a recording module, the operation unit, or a control unit.

AN: The device according to any of paragraphs AJ-AM, wherein: the device further comprises a reference-frequency generator configured to: detect RF excitation; and provide a reference frequency matching the RF excitation; and the control unit is configured to: modulate the data using the reference frequency as a carrier frequency to provide a modulated signal; and transmit the modulated signal via the at least one of the one or more antennas.

AO: A method, comprising, by a control unit of an electrophysiological (EP) measurement device: detecting a first change in a magnetic field around the device; subsequently, detecting commencement of a quiescent period of the magnetic field; during the quiescent period, measuring a subject to provide an EP signal; determining a readout period of a magnetic-resonance (MR) system; determining a modulated signal based at least in part on the EP signal; and transmitting the modulated signal to the MR system during the readout period.

AP: The method according to paragraph AO, further comprising, by the control unit: after measuring the subject, detecting a second change in the magnetic field around the device; and determining the readout period commencing with the second change.

AQ: The method according to paragraph AO or AP, further comprising, by the control unit: detecting a third change in the magnetic field around the device; and determining the readout period commencing a predetermined time after the third change.

AR: The method according to any of paragraphs AO-AQ, further comprising, by the control unit: detecting a fourth change in the magnetic field around the device; subsequently, detecting commencement of a second quiescent period of the magnetic field; and determining the readout period comprising a time period within the second quiescent period.

AS: The method according to any of paragraphs AO-AR, further comprising, by the control unit: determining a trigger point based at least in part on the EP signal, the trigger point associated with a physiological event of the subject; determining a second modulated signal indicating the trigger point; and transmitting the second modulated signal to the MR system during the readout period.

AT: The method according to any of paragraphs AO-AS, further comprising, by the control unit: detecting a second change in the magnetic field around the device; decoding a control signal from the second change in the magnetic field, the control signal indicating a carrier frequency; and determining the modulated signal by modulating the EP signal substantially at the carrier frequency.

AU: A method, comprising, by a control unit of an electrophysiological (EP) stimulation device: detecting a first change in a magnetic field around the device; subsequently, detecting commencement of a quiescent period of the magnetic field; determining that the quiescent period is not a readout period of a magnetic-resonance (MR) system; and during the quiescent period, providing a stimulus to tissues of a subject, the stimulus comprising at least one of electrical current or electromagnetic radiation.

AV: The method according to paragraph AU, further comprising, by the control unit: during the quiescent period, measuring the subject to provide an EP signal; determining a first readout period of the MR system; and determining a modulated signal based at least in part on the EP signal; and transmitting the modulated signal to the MR system during the first readout period.

AW: The method according to paragraph AU or AV, further comprising, by the control unit: detecting a second change in the magnetic field around the device; decoding a control signal from the second change in the magnetic field; and providing the stimulus based at least in part on the control signal.

AX: A computer-readable medium, e.g., a computer storage medium, having thereon computer-executable instructions, the computer-executable instructions upon execution configuring a computer to perform operations as any of paragraphs AC-AI, AJ-AN, AO-AT, or AU-AW recites.

AY: A device comprising: a processor; and a computer-readable medium, e.g., a computer storage medium, having thereon computer-executable instructions, the computer-executable instructions upon execution by the processor configuring the device to perform operations as any of paragraphs AC-AI, AJ-AN, AO-AT, or AU-AW recites.

AZ: A system comprising: means for processing; and means for storing having thereon computer-executable instructions, the computer-executable instructions including means to configure the system to carry out a method as any of paragraphs AC-AI, AJ-AN, AO-AT, or AU-AW recites.

### CONCLUSION

In view of the foregoing, various aspects permit integrated MRI imaging and EP analysis. Some prior schemes are bulky, expensive (>$200k for 64-ch device), and provide low quality measurements because of EMI. By contrast, some examples herein include devices that are small and inexpensive. Some example devices can be mass produced using silicon fabrication techniques. Some example devices are easy to set up inside the MRI scanner. Some example devices can provide 512 channels of neural recording and stimulation for ˜$300. Some example devices are reusable and communicate wirelessly, so can have reduced size compared to prior schemes. Some example devices do not require a bulky amplifier. Some example devices do not require putting an amplifier inside the MRI scanning room. Some example devices can provide >128 channels of EEG within the MRI bore. Some example devices can provide 1000 channels of stimulation or detection. Some examples can be integrated within MRI machines, e.g., to provide a multimodal imaging system that captures MRI and EP data. Some example devices can measure the brain, other organs, or other tissues.

The word “or” and the phrase “and/or” are used herein in an inclusive sense unless specifically stated otherwise. Accordingly, conjunctive language such as, but not limited to, at least one of the phrases “X, Y, or Z,” “at least X, Y, or Z,” “at least one of X, Y or Z,” “one or more of X, Y, or Z,” and/or any of those phrases with “and/or” substituted for “or,” unless specifically stated otherwise, is to be understood as signifying that an item, term, etc. can be either X, or Y, or Z, or a combination of any elements thereof (e.g., a combination of XY, XZ, YZ, and/or XYZ). Any use herein of phrases such as “X, or Y, or both” or “X, or Y, or combinations thereof” is for clarity of explanation and does not imply that language such as “X or Y” excludes the possibility of both X and Y, unless such exclusion is expressly stated.

As used herein, language such as “one or more Xs” shall be considered synonymous with “at least one X” unless otherwise expressly specified. Any recitation of “one or more Xs” signifies that the described steps, operations, structures, or other features may, e.g., include, or be performed with respect to, exactly one X, or a plurality of Xs, in various examples, and that the described subject matter operates regardless of the number of Xs present, as long as that number is greater than or equal to one.

Conditional language such as, among others, “can,” “could,” “might” or “may,” unless specifically stated otherwise, are understood within the context to present that certain examples include, while other examples do not include, certain features, elements and/or steps. Thus, such conditional language is not generally intended to imply that certain features, elements and/or steps are in any way required for one or more examples or that one or more examples necessarily include logic for deciding, with or without user input or prompting, whether certain features, elements and/or steps are included or are to be performed in any particular example.

Although some features and examples herein have been described in language specific to structural features and/or methodological steps, it is to be understood that the appended claims are not necessarily limited to the specific features or steps described herein. Rather, the specific features and steps are disclosed as preferred forms of implementing the claimed invention. For example, network 1750, processor 1786, and other structures described herein for which multiple types of implementing devices or structures are listed can include any of the listed types, and/or multiples and/or combinations thereof.

Moreover, this disclosure is inclusive of combinations of the aspects described herein. References to “a particular aspect” (or “embodiment” or “version”) and the like refer to features that are present in at least one aspect of the invention. Separate references to “an aspect” (or “embodiment”) or “particular aspects” or the like do not necessarily refer to the same aspect or aspects; however, such aspects are not mutually exclusive, unless so indicated or as are readily apparent to one of skill in the art. The use of singular or plural in referring to “method” or “methods” and the like is not limiting.

It should be emphasized that many variations and modifications can be made to the above-described examples, the elements of which are to be understood as being among other acceptable examples. All such modifications and variations are intended to be included herein within the scope of this disclosure and protected by the following claims. Moreover, in the claims, any reference to a group of items provided by a preceding claim clause is a reference to at least some of the items in the group of items, unless specifically stated otherwise. This document expressly envisions alternatives with respect to each and every one of the following claims individually, in any of which claims any such reference refers to each and every one of the items in the corresponding group of items. Furthermore, in the claims, unless otherwise explicitly specified, an operation described as being “based on” a recited item can be performed based on only that item, or based at least in part on that item. This document expressly envisions alternatives with respect to each and every one of the following claims individually, in any of which claims any “based on” language refers to the recited item(s), and no other(s).

Some operations of example processes are illustrated in individual blocks and summarized with reference to those blocks. The processes are illustrated as logical flows of blocks, each block of which can represent one or more operations that can be implemented in hardware, software, or a combination thereof. In the context of software, the operations represent computer-executable instructions stored on one or more computer-readable media that, when executed by one or more processors, enable the one or more processors to perform the recited operations. Generally, computer-executable instructions include routines, programs, objects, modules, components, data structures, and the like that perform particular functions or implement particular abstract data types. The order in which the operations are described is not intended to be construed as a limitation, and any number of the described operations can be executed in any order, combined in any order, subdivided into multiple sub-operations, or executed in parallel to implement the described processes.

Accordingly, the methods, processes, or operations described above can be embodied in, and fully automated via, software code modules executed by one or more computers or processors. As used herein, the term “module” is intended to represent example divisions of the described operations (e.g., implemented in software or hardware) for purposes of discussion, and is not intended to represent any type of requirement or required method, manner or organization. Therefore, while various “modules” are discussed herein, their functionality and/or similar functionality can be arranged differently (e.g., combined into a smaller number of modules, broken into a larger number of modules, etc.). In some instances, the functionality and/or modules discussed herein may be implemented as part of a computer operating system (OS). In other instances, the functionality and/or modules may be implemented as part of a device driver, firmware, application, or other software subsystem.

Example computer-implemented operations described herein can additionally or alternatively be embodied in specialized computer hardware, e.g., sensing units for use in the MRI environment; wireless EEG electrodes; or signal filters. For example, various aspects herein may take the form of an entirely hardware aspect, an entirely software aspect (including firmware, resident software, micro-code, etc.), or an aspect combining software and hardware aspects. These aspects can all generally be referred to herein as a “service,” “circuit,” “circuitry,” “module,” or “system.” The described processes can be performed by resources associated with one or more computing systems 1701, 1702 or processors 1786, such as one or more internal or external CPUs or GPUs, or one or more pieces of hardware logic such as FPGAs, DSPs, or other types of accelerators.

1. A system, comprising:
one or more antennas;
a reference unit comprising:
a reference electrode configured to contact the body of a subject and to provide a signal;
a signal transmission unit configured to transmit the signal as two differential signals via a differential pair; and
a converter configured to receive the two differential signals via the differential pair and to provide a reconstructed reference signal based at least in part on the two differential signals;
measurement circuitry configured to measure an electrophysiological (EP) signal of the subject based at least in part on the reconstructed reference signal;
detection circuitry configured to:
detect, using at least one of the one or more antennas, magnetic-field changes due to the operation of magnetic resonance (MR) coil(s); and
isolate the detection circuitry from electrical transients during the magnetic-field changes;
a control unit configured to:
operate the detection circuitry to measure the EP signal at a time other than during the magnetic-field changes; and
a communication module configured to:
transmit data corresponding to the EP signal via at least one of the one or more antennas.

2. The system according to claim 1, further comprising:
a programmable stimulation module configured to provide at least one of electrical current or electromagnetic radiation to tissues of a subject.

3. The system according to claim 2, wherein the control unit is configured to operate the programmable stimulation module to provide the at least one of electrical current or electromagnetic radiation at a time other than during the magnetic field changes.

4. The system according to claim 1, wherein the programmable stimulation module is configured to provide the at least one of electrical current or electromagnetic radiation corresponding with a predetermined stimulation pattern.

5. The system according claim 1, further comprising:
a wireless power harvesting module configured to:
receive electromagnetic energy via at least one of the one or more antennas;
transform the received electromagnetic energy to electrical energy; and
provide the electrical energy to at least one other component of the device to power the at least one other component of the device, wherein the at least one other component comprises at least one of a stimulation module, a recording module, the reference unit, the detection circuitry, the measurement circuitry, the control unit, or the communication module.

6. The system according to claim 1, wherein the detection circuitry comprises a variable gain amplifier and the detection circuitry is configured to reduce the gain during the operation of the MRI coil(s).

7. The system according to claim 1, wherein the measurement circuitry:
comprises at least one active electrode configured to contact the body of the subject and to provide an active signal; and
is configured to provide the EP signal based on the reconstructed reference signal and the active signal.

8. A device, comprising:
one or more antennas;
an operation unit comprising at least one of an electrophysiological (EP) detection unit or a stimulation unit; and
a control unit configured to:
detect changes to a magnetic field around the device;
isolate the operation unit from transients during the magnetic-field changes; and
activate the operation unit at a time other than during the magnetic-field changes.

9. The device according to claim 8, wherein:
the operation unit comprises the EP detection unit configured to, when activated, measure an electrophysiological (EP) signal of a subject; and
the control unit is further configured to:
detect a readout period based at least in part on the changes to the magnetic field; and
transmit data corresponding to the electrophysiological signal via at least one of the one or more antennas during the readout period.

10. The device according to claim 8, wherein:
the operation unit comprises the stimulation unit configured to, when activated, provide at least one of electrical current or electromagnetic radiation to tissues of a subject.

11. The device according to claim 8, further comprising:
a wireless power harvesting module configured to:
receive electromagnetic energy within the MR bore;
transform the received electromagnetic energy to electrical energy; and
provide the electrical energy to at least one other component of the device to power the at least one other component, wherein the at least one other component comprises at least one of a stimulation module, a recording module, the operation unit, or a control unit.

12. The device according to claim 8, wherein:
the device further comprises a reference-frequency generator configured to:
detect RF excitation; and
provide a reference frequency matching the RF excitation; and
the control unit is configured to:
modulate the data using the reference frequency as a carrier frequency to provide a modulated signal; and
transmit the modulated signal via the at least one of the one or more antennas.

13. A method, comprising, by a control unit of an electrophysiological (EP) measurement device:
detecting a first change in a magnetic field around the device;
subsequently, detecting commencement of a quiescent period of the magnetic field;
during the quiescent period, measuring a subject to provide an EP signal;
determining a readout period of a magnetic-resonance (MR) system;
determining a modulated signal based at least in part on the EP signal; and
transmitting the modulated signal to the MR system during the readout period.

14. The method according to claim 13, further comprising, by the control unit:
after measuring the subject, detecting a second change in the magnetic field around the device; and
determining the readout period commencing with the second change.

15. The method according to claim 13, further comprising, by the control unit:
detecting a third change in the magnetic field around the device; and
determining the readout period commencing a predetermined time after the third change.

16. The method according to claim 13, further comprising, by the control unit:
detecting a fourth change in the magnetic field around the device;
subsequently, detecting commencement of a second quiescent period of the magnetic field; and
determining the readout period comprising a time period within the second quiescent period.

17. The method according to claim 13, further comprising, by the control unit:
determining a trigger point based at least in part on the EP signal, the trigger point associated with a physiological event of the subject;
determining a second modulated signal indicating the trigger point; and
transmitting the second modulated signal to the MR system during the readout period.

18. The method according to claim 13, further comprising, by the control unit:
detecting a second change in the magnetic field around the device;
decoding a control signal from the second change in the magnetic field, the control signal indicating a carrier frequency; and
determining the modulated signal by modulating the EP signal substantially at the carrier frequency.

19. A method, comprising, by a control unit of an electrophysiological (EP) stimulation device:
detecting a first change in a magnetic field around the device;
subsequently, detecting commencement of a quiescent period of the magnetic field;
determining that the quiescent period is not a readout period of a magnetic-resonance (MR) system; and
during the quiescent period, providing a stimulus to tissues of a subject, the stimulus comprising at least one of electrical current or electromagnetic radiation.

20. The method according to claim 19, further comprising, by the control unit:
during the quiescent period, measuring the subject to provide an EP signal;
determining a first readout period of the MR system; and
determining a modulated signal based at least in part on the EP signal; and
transmitting the modulated signal to the MR system during the first readout period.

21. The method according to claim 19, further comprising, by the control unit:
detecting a second change in the magnetic field around the device;
decoding a control signal from the second change in the magnetic field; and
providing the stimulus based at least in part on the control signal.
